(19) United States
(12) Patent Application Publication
(10) Pub. No.: US 2022/0064668 A1
(43) Pub. Date: Mar. 3, 2022

(54) MODIFIED ADENO-ASSOCIATED VIRAL VECTORS FOR USE IN GENETIC ENGINEERING

(71) Applicant: Intima Bioscience, Inc., New York, NY (US)

(72) Inventors: Modassir CHOUDHRY, New York, NY (US); Mavis AGBANDJE-MCKENNA, New York, NY (US); Tilmann BUERCKSTUEMMER, New York, NY (US); Thomas HENLEY, New York, NY (US); Lydia VINEY, New York, NY (US)

(21) Appl. No.: 17/365,026

(22) Filed: Jul. 1, 2021

Related U.S. Application Data
(63) Continuation of application No. PCT/US2019/067495, filed on Dec. 19, 2019.
(60) Provisional application No. 62/788,109, filed on Jan. 3, 2019.
(60) Provisional application No. 62/787,721, filed on Jan. 2, 2019.

(51) Int. Cl. C12N 15/86

(52) U.S. Cl. CPC C12N 15/86; C12N 2750/14152; C12N 2750/14134; C12N 2750/14133

(57) ABSTRACT

Adeno-associated virus has numerous advantages for its use in gene therapy. The present disclosures provide genetically modified adeno-associated viral vectors, and the methods of making the genetically modified adeno-associated viral vectors and compositions in treating cancer, other conditions, diseases, and disorders.

### CROSS-REFERENCE TO RELATED APPLICATION

This application is a continuation of International Application No. PCT/US2019/067495, filed Dec. 19, 2019 which claims the benefit of U.S. Provisional Patent Application No. 62/787,721 filed on Jan. 2, 2019, and U.S. Provisional Patent Application No. 62/788,109 filed on Jan. 3, 2019, the disclosures of each of which are hereby incorporated by reference herein in their entirety.

### SEQUENCE LISTING

The instant application contains a Sequence Listing which has been submitted electronically in ASCII format and is hereby incorporated by reference in its entirety. Said ASCII copy, created on Jun. 30, 2021, is named 199827739301_SL.txt and is 196,556 bytes in size.

### BACKGROUND

Despite remarkable advances in cancer therapeutics over the last 50 years, there remain many tumor types that are recalcitrant to chemotherapy, radiotherapy or biotherapy, particularly in advanced stages that cannot be addressed through surgical techniques. Recently there have been significant advances in the genetic engineering of lymphocytes to recognize molecular targets on tumors in vivo, resulting in remarkable cases of remission of the targeted tumor. Recombinant adeno-associated viral (AAV) vectors, are advantageous for use in gene and cell therapy. For example, AAV vectors lack pathogenicity and are able to infect non-dividing cells. The increasing use of AAV vectors underscores the necessity of improving AAV vectors for better delivery of transgenes both in gene and cell therapy.

### SUMMARY

In one aspect, provided herein are polynucleic acid sequences that encode: (a) in a first reading frame, an adeno-associated virus (AAV) VP1 polypeptide, an AAV VP2 polypeptide, and an AAV VP3 polypeptide, and (b) in a second reading frame, a modified AAV assembly-activating protein (AAP) polypeptide that is at least partially in a region of said first reading frame that encodes at least a portion of said VP2 polypeptide and at least a portion of said VP3 polypeptide, and wherein said AAP polypeptide comprises i) at least one amino acid substitution in said region of said first reading frame that encodes at least a portion of said VP2 polypeptide as compared to a wild-type AAV AAP polypeptide of the same AAV serotype of said VP2 polypeptide; or ii) at least one amino acid substitution in said region of said first reading frame that encodes at least a portion of said VP3 polypeptide as compared to a wild-type AAV AAP polypeptide of the same AAV serotype of said VP3 polypeptide.

In some embodiments, one of said VP1, VP2, and VP3 polypeptides is a first AAV serotype, and one of said VP1, VP2, and VP3 polypeptides is a second AAV serotype, wherein said first and second AAV serotypes are different.

In some embodiments, introduction of a said polynucleic acid into a population of cells under conditions suitable for AAV particle production from said cells, results in a higher titer of AAV particles produced by said population of cells compared to introduction of a comparable polynucleic acid lacking said modified AAP polypeptide.

In some embodiments, said at least one amino acid substitution in said region of said first reading frame that encodes at least a portion of said VP2 polypeptide is in a helical region of said modified AAP polypeptide. In some embodiments, said at least one amino acid substitution in said region of said first reading frame that encodes at least a portion of said VP3 polypeptide is in a helical region of said modified AAP polypeptide

In some embodiments, said at least one amino acid substitution in said region of said first reading frame that encodes at least a portion of said VP2 polypeptide is in a helical region of said modified AAP polypeptide comprises one, two, three, four, five, six, seven, eight, nine, ten, eleven, twelve, or more substitutions; or wherein said at least one amino acid substitution in said region of said first reading frame that encodes at least a portion of said VP3 polypeptide is in a helical region of said modified AAP polypeptide comprises one, two, three, four, five, six, seven, eight, nine, ten, eleven, twelve, or more substitutions; or both.

In some embodiments, said VP2 polypeptide is an AAV6 serotype, and said at least one amino acid substitution in said region of said first reading frame that encodes at least a portion of said VP2 polypeptide is in a helical region of said modified AAP polypeptide is within amino acids 13 to 27 of said AAP polypeptide.

In some embodiments, said at least one amino acid substitution in said region of said first reading frame that encodes at least a portion of said VP2 polypeptide is in a helical region of said modified AAP polypeptide is within amino acids 21 to 27 of said AAP polypeptide.

In some embodiments, said at least one amino acid substitution comprises a substitution at amino acid K21, C22, L23, M24, M25, or R27, or any combination thereof, in said AAP polypeptide. In some embodiments, said at least one amino acid substitution comprises a substitution at amino acids K21, C22, L23, M24, M25, and R27 in said AAP polypeptide. In some embodiments, said at least one amino acid substitution comprises a K21L, a C22L, a L23W, a M24D, a M25L, or a R27Q substitution, or any combination thereof in said AAP polypeptide. In some embodiments, said at least one amino acid substitution comprises a K21L, a C22L, a L23W, a M24D, a M25L, and a R27Q substitution in said AAP polypeptide.

In some embodiments, said at least one amino acid substitution comprises a substitution at amino acid K53, C54, L55, M56, M57, and R59 of SEQ ID NO: 39, or any combination thereof, in said AAP polypeptide. In some embodiments, said at least one amino acid substitution comprises a substitution at amino acids K53, C54, L55, M56, M57, and R59 of SEQ ID NO: 39, in said AAP polypeptide. In some embodiments, said at least one amino acid substitution comprises a K53L, a C54L, a L55W, a M56D, a M57L, or a R59Q substitution in SEQ ID NO: 39, or any combination thereof, in said AAP polypeptide. In some embodiments, said at least one amino acid substitution comprises a K53L, a C54L, a L55W, a M56D, a M57L, and a R59Q substitution in SEQ ID NO: 39, in said AAP polypeptide.

In some embodiments, said polynucleic acid sequence comprises a nucleic acid sequence having at least 80%, 85%, 90%, 95%, 96%, 97%, 98%, 99%, or 100% identity to any one of SEQ ID NOs: 51-65. In some embodiments, said polynucleic acid sequence comprises a nucleic acid sequence that encodes a polypeptide sequence having at least 80%, 85%, 90%, 95%, 96%, 97%, 98%, 99%, or 100% identity to any one of any one of SEQ ID NOs: 44-50. In some embodiments, said polynucleic acid sequence comprises a nucleic acid sequence having at least 80%, 85%, 90%, 95%, 96%, 97%, 98%, 99%, or 100% identity to any one of SEQ ID NOs: 3-15. In some embodiments, said polynucleic acid sequence comprises a nucleic acid sequence that encodes a polypeptide sequence having at least 80%, 85%, 90%, 95%, 96%, 97%, 98%, 99%, or 100% identity to any one of any one of SEQ ID NOs: 2 or 16-25.

In some embodiments, said first AAV serotype and said second AAV serotype are selected from AAV1, AAV2, AAV3, AAV4, AAV5, AAV6, AAV7, AAV8, AAV9, AAV10, AAV11, AAV12.

In some embodiments, said first AAV serotype is AAV12 and said second AAV serotype is AAV6. In some embodiments, said VP1 and VP2 polypeptides are AAV12 serotype and said VP3 polypeptide is an AAV6 serotype.

In one aspect, provided herein are polynucleic acid sequences that encode i) in a first reading frame, a VP2 polypeptide of a predetermined AAV serotype, and ii) in a second reading frame, a modified assembly-activating protein (AAP) polypeptide comprising at least one amino acid substitution within amino acids 5-40 in said modified AAP polypeptide with respect to a wild type AAP polypeptide of said predetermined AAV serotype.

In some embodiments, said polynucleic acid sequence comprises a nucleic acid sequence encoding an AAV12 VP1 polypeptide, a nucleic acid sequence encoding an AAV12 VP2 polypeptide, and a nucleic acid sequence encoding an AAV6 VP3 polypeptide, in a single reading frame.

In some embodiments, said at least one amino acid substitution comprises a substitution at amino acid K21, C22, L23, M24, M25, or R27, or any combination thereof, in said AAP polypeptide. In some embodiments, said at least one amino acid substitution comprises a substitution at amino acids K21, C22, L23, M24, M25, and R27 in said AAP polypeptide. In some embodiments, said at least one amino acid substitution comprises a K21L, a C22L, a L23W, a M24D, a M25L, or a R27Q substitution, or any combination thereof, in said AAP polypeptide. In some embodiments, said at least one amino acid substitution comprises a K21L, a C22L, a L23W, a M24D, a M25L, and a R27Q substitution in said AAP polypeptide.

In some embodiments, said at least one amino acid substitution comprises a substitution at amino acid K53, C54, L55, M56, M57, and R59 of SEQ ID NO: 39, or any combination thereof, in said AAP polypeptide. In some embodiments, said at least one amino acid substitution comprises a substitution at amino acids K53, C54, L55, M56, M57, and R59 of SEQ ID NO: 39, in said AAP polypeptide. In some embodiments, said at least one amino acid substitution comprises a K53L, a C54L, a L55W, a M56D, a M57L, or a R59Q substitution in SEQ ID NO: 39, or any combination thereof, in said AAP polypeptide. In some embodiments, said at least one amino acid substitution comprises a K53L, a C54L, a L55W, a M56D, a M57L, and a R59Q substitution in SEQ ID NO: 39, in said AAP polypeptide.

In some embodiments, said polynucleic acid sequence comprises a nucleic acid sequence having at least 80%, 85%, 90%, 95%, 96%, 97%, 98%, 99%, or 100% identity to any one of SEQ ID NOs: 51-65. In some embodiments, said polynucleic acid sequence comprises a nucleic acid sequence that encodes a polypeptide sequence having at least 80%, 85%, 90%, 95%, 96%, 97%, 98%, 99%, or 100% identity to any one of any one of SEQ ID NOs: 44-50. In some embodiments, said polynucleic acid sequence comprises a nucleic acid sequence having at least 80%, 85%, 90%, 95%, 96%, 97%, 98%, 99%, or 100% identity to any one of SEQ ID NOs: 3-15. In some embodiments, said polynucleic acid sequence comprises a nucleic acid sequence that encodes a polypeptide sequence having at least 80%, 85%, 90%, 95%, 96%, 97%, 98%, 99%, or 100% identity to any one of any one of SEQ ID NOs: 16-25.

In some embodiments, said predetermined AAV serotype is AAV6.

In some embodiments, introduction of a said polynucleic acid into a population of cells under conditions suitable for AAV particle production from said cells, results in a higher titer of AAV particles produced by said population of cells compared to introduction of a comparable polynucleic acid lacking said modified AAP polypeptide.

In one aspect, provided herein are polynucleic acid sequences encoding an adeno-associated virus (AAV) VP1 polypeptide, an AAV VP2 polypeptide, an AAV VP3 polypeptide, and a modified AAV AAP polypeptide, and wherein said modified AAP polypeptide comprises at least one amino acid substitution as compared to a wild-type AAP polypeptide.

In some embodiments, two of said VP1, VP2, and VP3 polypeptides are a first AAV serotype, and one of said VP1, VP2, and VP3 polypeptides is a second AAV serotype, wherein said first AAV serotype and said second AAV serotype are different.

In some embodiments, said modified AAP polypeptide comprises at least one amino acid substitution as compared to a wild-type AAP polypeptide of said first AAV serotype or said second AAV serotype.

In some embodiments, introduction of a said polynucleic acid into a population of cells under conditions suitable for AAV particle production from said cells, results in a higher titer of AAV particles produced by said population of cells compared to introduction of a comparable polynucleic acid lacking said modified AAP polypeptide.

In some embodiments, said at least one amino acid substitution is in a helical region of said modified AAP polypeptide.

In some embodiments, said at least one amino acid substitution comprises one, two, three, four, five, six, seven, eight, nine, ten, eleven, twelve, or more amino acid substitutions.

In some embodiments, said at least one amino acid substitution is within amino acids 13 to 27 of said modified AAP polypeptide. In some embodiments, said at least one amino acid substitution is within amino acids 21 to 27 of said AAP polypeptide.

In some embodiments, said at least one amino acid substitution comprises a substitution at amino acid K21, C22, L23, M24, M25, or R27, or any combination thereof, in said AAP polypeptide. In some embodiments, said at least one amino acid substitution comprises a substitution at amino acids K21, C22, L23, M24, M25, and R27 in said AAP polypeptide. In some embodiments, said at least one amino acid substitution comprises a K21L, a C22L, a L23W, a M24D, a M25L, or a R27Q substitution, and any combination thereof, in said modified AAP polypeptide. In some embodiments, said at least one substitution comprises a K21L, a C22L, a L23W, a M24D, a M25L, and a R27Q substitution in said modified AAP polypeptide.

In some embodiments, said at least one amino acid substitution comprises a substitution at amino acid K53, C54, L55, M56, M57, and R59 of SEQ ID NO: 39, or any combination thereof, in said AAP polypeptide. In some embodiments, said at least one amino acid substitution comprises a substitution at amino acids K53, C54, L55, M56, M57, and R59 of SEQ ID NO: 39, in said AAP polypeptide. In some embodiments, said at least one amino acid substitution comprises a K53L, a C54L, a L55W, a M56D, a M57L, or a R59Q substitution in SEQ ID NO: 39, or any combination thereof, in said AAP polypeptide. In some embodiments, said at least one amino acid substitution comprises a K53L, a C54L, a L55W, a M56D, a M57L, and a R59Q substitution in SEQ ID NO: 39, in said AAP polypeptide.

In some embodiments, said polynucleic acid sequence comprises a nucleic acid sequence having at least 80%, 85%, 90%, 95%, 96%, 97%, 98%, 99%, or 100% identity to any one of SEQ ID NOs: 51-65. In some embodiments, said polynucleic acid sequence comprises a nucleic acid sequence that encodes a polypeptide sequence having at least 80%, 85%, 90%, 95%, 96%, 97%, 98%, 99%, or 100% identity to any one of any one of SEQ ID NOs: 44-50. In some embodiments, said polynucleic acid sequence comprises a nucleic acid sequence having at least 80%, 85%, 90%, 95%, 96%, 97%, 98%, 99%, or 100% identity to any one of SEQ ID NOs: 3-15. In some embodiments, said polynucleic acid sequence comprises a nucleic acid sequence that encodes a polypeptide sequence having at least 80%, 85%, 90%, 95%, 96%, 97%, 98%, 99%, or 100% identity to any one of any one of SEQ ID NOs: 2 or 16-25.

In some embodiments, said VP2 polypeptide is an AAV6 serotype. In some embodiments, said first AAV serotype and said second AAV serotype are selected from AAV1, AAV2, AAV3, AAV4, AAV5, AAV6, AAV7, AAV8, AAV9, AAV10, AAV11, AAV12. In some embodiments, said first AAV serotype is AAV12 and said second AAV serotype is AAV6. In some embodiments, said VP1 polypeptide is an AAV12 serotype, said VP2 polypeptide is an AAV12 serotype, and said VP3 polypeptide is an AAV6 serotype.

In some embodiments, said polynucleic acid sequence comprises a sequence with at least 80%, 85%, 90%, 95%, 96%, 97%, 98%, 99%, or 100% identity to SEQ ID NO: 51-65. In some embodiments, said polynucleic acid sequence comprises a sequence that encodes a protein with at least 80%, 85%, 90%, 95%, 96%, 97%, 98%, 99%, or 100% identity to SEQ ID NO: 44-50. In some embodiments, said polynucleic acid sequence comprises a sequence with at least 80%, 85%, 90%, 95%, 96%, 97%, 98%, 99%, or 100% identity to SEQ ID NO: 3-15. In some embodiments, said polynucleic acid sequence comprises a sequence that encodes a protein with at least 80%, 85%, 90%, 95%, 96%, 97%, 98%, 99%, or 100% identity to SEQ ID NO: 2 or 16-25.

In some embodiments, said AAP polypeptide encodes a functional AAP polypeptide.

In one aspect, provided herein are isolated polypeptide sequences encoded by a polynucleic acid sequence described herein.

In one aspect, provided herein are populations of cells that comprise said polynucleic acid sequence described herein. In some embodiments, the populations of cells are produced by transfecting cells with said polynucleic acid sequence described herein. In some embodiments, said population of cells produces AAV particles. In some embodiments, said AAV particles comprise said polynucleic acid sequence of any one of claims 1-56. In some embodiments, said AAV particles comprise each of said polypeptides encoded by said polynucleic acid sequence of any one of claims 1-58.

In one aspect, provided herein are methods of making AAV particles, said method comprising introducing said polynucleic acid sequence described herein, culturing said cells for a sufficient time for said cells to produce a population of AAV particles, wherein a titer of said produced population of AAV particles is higher compared to a titer of AAV particles produced by introducing a comparable polynucleic acid that does not comprise said modified AAP polypeptide.

In one aspect, provided herein are a plurality of isolated AAV particles produced by a method described herein.

In one aspect, provided herein are compositions comprising the plurality of isolated AAV particles that comprise said polynucleic acid described herein. In some embodiments, said composition is in a unit dosage form. In some embodiments, said composition is cryopreserved.

In one aspect, provided herein are systems comprising a first polynucleic acid sequence that encodes at least three adeno-associated virus (AAV) polypeptides, wherein said first polynucleic acid sequence encodes a VP1 polypeptide, a VP2 polypeptide, and a VP3 polypeptide, wherein two of said VP1, VP2, and VP3 polypeptides are from a first AAV serotype, and one of said VP1, VP2, and VP3 polypeptides is from a second AAV serotype, wherein said first AAV serotype and said second AAV serotype are not the same; and a second polynucleic acid sequence heterologous to said first polynucleic acid sequence that encodes an AAV assembly-activating protein (AAP) polypeptide, wherein said first polynucleic acid sequence and second polynucleic acid sequence are not covalently linked.

In some embodiments, introduction of a said polynucleic acid into a population of cells under conditions suitable for AAV particle production from said cells, results in a higher titer of AAV particles produced by said population of cells compared to introduction of a comparable polynucleic acid lacking said modified AAP polypeptide.

In some embodiments, said AAV AAP polypeptide is a wild-type AAV AAP polypeptide. In some embodiments, said AAV AAP polypeptide is an AAV6 AAP polypeptide. In some embodiments, said first AAV serotype and said second AAV serotype are selected from AAV1, AAV2, AAV3, AAV4, AAV5, AAV6, AAV7, AAV8, AAV9, AAV10, AAV11, AAV12, or any combination thereof. In some embodiments, said first AAV serotype is AAV12. In some embodiments, said first AAV serotype is AAV12 and said second AAV serotype is AAV6. In some embodiments, said first polynucleic acid sequence encodes an AAV12 VP1, an AAV12, VP2 and an AAV6 VP3.

In some embodiments, said polynucleic acid sequence comprises a sequence with at least 80%, 85%, 90%, 95%, 96%, 97%, 98%, 99%, or 100% identity to SEQ ID NO: 51-65. In some embodiments, said polynucleic acid sequence comprises a sequence that encodes a protein with at least 80%, 85%, 90%, 95%, 96%, 97%, 98%, 99%, or 100% identity to SEQ ID NO: 44-50. In some embodiments, said polynucleic acid sequence comprises a sequence with at least 80%, 85%, 90%, 95%, 96%, 97%, 98%, 99%, or 100% identity to SEQ ID NO: 3-15. In some embodiments, said polynucleic acid sequence comprises a sequence that encodes a protein with at least 80%, 85%, 90%, 95%, 96%, 97%, 98%, 99%, or 100% identity to SEQ ID NO: 2 or 16-25.

In one aspect, provided herein are populations of cells that comprise said system described herein. In some embodiments, the population of cells is produced by transfecting cells with a system described herein. In some embodiments, said population of cells produce AAV particles. In some embodiments, said AAV particles comprise a system described herein. In some embodiments, said AAV particles comprise each of said polypeptides encoded by said system of any one of claims 68-79.

In one aspect, provided herein are methods of making AAV particles, said method comprising introducing a system described herein, culturing said cells for a sufficient time for said cells to produce a population of AAV particles, wherein a titer of said produced population of AAV particles is higher compared to a titer of AAV particles produced by introducing a comparable system that does not comprise said heterologous AAP polypeptide. In one aspect, provided herein is a plurality of isolated AAV particles produced by a method described herein.

In one aspect, provided herein are methods of making a population of engineered cells, said method comprising contacting a plurality of cells with a plurality of AAV particles that comprise a polynucleic acid sequence described herein, wherein said plurality of AAV particles further comprise a transgene, and culturing the plurality of cells for a time sufficient to express said transgene.

In some embodiments, said transgene is integrated into the genome of said plurality of cells.

In some embodiments, said transgene comprises homology arms capable of mediating targeted integration of said transgene into the genome of said plurality of cells.

In some embodiments, said method further comprises introducing a DNA endonuclease or a nucleic acid encoding said DNA endonuclease.

In some embodiments, said DNA endonuclease mediates a double strand break in the genome of said plurality of cells.

In some embodiments, said transgene is integrated into the genome of said cells with an efficiency of at least 30%, 40%, 50%, 60%, 70%, 80%, 90%, 95%, or 99%.

In some embodiments, said transgene is integrated into the genome of at least 30%, 40%, 50%, 60%, 70%, 80%, 90%, 95%, or 99% of said in said plurality, in the absence of a selection step.

In one aspect, provided herein are populations of cells produced by a method described herein. In some embodiments, said cells are administered to a subject. In some embodiments, said subject has cancer.

In one aspect, provided herein are methods of making a population of genetically modified cells, said method comprising: obtaining a population of cells from a subject; introducing an adeno-associated virus (AAV) vector that comprises a transgene into said population of cells, wherein said AAV vector comprises a polynucleic acid sequence described herein, and wherein said transgene is integrated into the genome of said population of cells, to thereby produce a population of genetically modified cells In some embodiments, said population of cells comprises immune cells. In some embodiments, said population of immune cells comprises lymphocytes (e.g., T cells (e.g., CD8+ T cell, CD4+ T cell), tumor infiltrating lymphocytes, NK cells, NK T cells, B cells). In some embodiments, said population of cells comprises a population of primary cells. In some embodiments, said population of cells comprises ex vivo cells.

In some embodiments, the method further comprises introducing a clustered regularly interspaced short palindromic repeats (CRISPR) system into said population of cells, wherein said CRISPR system comprises i) a polynucleotide encoding an endonuclease or a polypeptide encoding an endonuclease; and ii) a guide ribonucleic acid (gRNA); wherein said polynucleotide encoding said endonuclease or said polypeptide encoding an endonuclease introduces an alteration in a gene sequence in a plurality of cells of said population, wherein said genomic alteration suppresses expression of said gene, and wherein said first gRNA comprises a sequence that binds a nucleic acid sequence of said gene.

In some embodiments, said genomic alteration results from a double strand break introduced by said CRISPR system. In some embodiments, said CRISPR system is introduced into said population of cells via transfection (e.g., electroporation).

In one aspect, provided herein are infectious recombinant chimeric adeno-associated virus (rAAV) particles comprising: a modified AAV AAP protein that comprises at least one amino acid substitution relative to a wild-type AAV AAP protein. In some embodiments, said particle comprises a chimeric capsid that comprises a VP1 protein, a VP2 protein, and a VP3 protein, wherein one of said VP1, VP2, and VP3 proteins are from a first AAV serotype, and one of said VP1, VP2, and VP3 proteins is from a second AAV serotype, wherein said first and second AAV serotypes are not the same. In some embodiments, a modified AAV AAP protein that comprises at least one amino acid substitution relative to a wild-type AAV AAP protein of either said first AAV serotype or said second AAV serotype. In some embodiments, said rAAV particle exhibits increased infectivity of a primary T cell relative to a comparable AAV particle that comprises said wild type AAV AAP protein and does not comprise said modified AAP protein. In some embodiments, infectivity is expressed as a ratio of infectious viral particles to total viral particles. In some embodiments, said particle comprises a transgene (heterologous nucleic acid). In some embodiments, said infectivity is at least 2, 3, 4, 5, 10, 50, 100, 500, 1000, or 10000 fold higher relative to a comparable AAV particle that comprises said wild type AAV AAP protein and does not comprise said modified AAP protein.

In some aspects, the present disclosure provides a nucleic acid that comprises an adeno-associated virus (AAV) nucleotide sequence comprising VP1, VP2, and VP3 sequences, wherein two of said VP1, VP2, and VP3 sequences are from a first AAV serotype, and one of said VP1, VP2, and VP3 sequence is from a second AAV serotype, wherein said AAV nucleotide sequence comprises a first assembly-activating protein (AAP) region within said VP2 sequence and a second AAP region within said VP3 sequence, and wherein said AAV nucleotide sequence comprises: (a) at least one mutation in said first AAP region, wherein said at least one mutation is with respect to the serotype of the VP2 sequence; or (b) at least one mutation in said second AAP region, wherein said at least one mutation is with respect to the serotype of the VP3 sequence.

In some embodiments, said first and second AAP regions increase titer of an AAV comprising said nucleic acid sequence as compared to a corresponding AAV comprising a comparable nucleic acid sequence without said first and second AAP regions. In some embodiments, said at least one mutation is in a helical region of an AAP polypeptide encoded by said first and second AAP regions. In some embodiments, said at least one mutation comprises one, two, three, four, five, six, seven, eight, nine, ten, eleven, twelve, or more mutations. In some embodiments, said at least one mutation comprises six mutations. In some embodiments, said at least one mutation is within the first 1, 2, 3, 4, 5, 6, 7, 8, 9, 10, 11, 12, 13, 14, 15, 16, 17, 8, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids of an AAV6 AAP polypeptide encoded by said AAP region, or in a corresponding region of a non-AAV6 AAP polypeptide. In some embodiments, said at least one mutation is within a region encoding amino acids 13 to 27 of an AAV6 AAP polypeptide encoded by said AAP region, or in a corresponding region of a non-AAV6 AAP polypeptide. In some embodiments, said at least one mutation is within a region encoding amino acids 21 to 27 of an AAV6 AAP polypeptide encoded by said AAP region, or within a corresponding region of a non-AAV6 AAP polypeptide. In some embodiments, said at least one mutation encodes K21L, C22L, L23W, M24D, M25L, and R27Q substitutions in said AAP polypeptide.

In some embodiments, said at least one amino acid substitution comprises a substitution at amino acid K53, C54, L55, M56, M57, and R59 of SEQ ID NO: 39, or any combination thereof, in said AAP polypeptide. In some embodiments, said at least one amino acid substitution comprises a substitution at amino acids K53, C54, L55, M56, M57, and R59 of SEQ ID NO: 39, in said AAP polypeptide. In some embodiments, said at least one amino acid substitution comprises a K53L, a C54L, a L55W, a M56D, a M57L, or a R59Q substitution in SEQ ID NO: 39, or any combination thereof, in said AAP polypeptide. In some embodiments, said at least one amino acid substitution comprises a K53L, a C54L, a L55W, a M56D, a M57L, and a R59Q substitution in SEQ ID NO: 39, in said AAP polypeptide.

In some embodiments, said first AAV serotype and said second AAV serotype are selected from AAV1, AAV2, AAV3, AAV4, AAV5, AAV6, AAV7, AAV8, AAV9, AAV10, AAV11, AAV12, or any combination thereof. In some embodiments, said first AAV serotype is AAV12 and said second AAV serotype is AAV6. In some embodiments, said VP1 and VP2 sequences are AAV12 sequences and said VP3 sequence is an AAV6 sequence. In some embodiments, after introduction into a plurality of cells, said nucleic acid confers an increased expression of a transgene as compared to a wild-type AAV nucleic acid.

In some aspects, the present disclosure provides a nucleic acid that comprises an adeno-associated virus (AAV) nucleotide sequence comprising a VP2 sequence of a predetermined serotype and an assembly-activating protein (AAP) nucleotide sequence comprising a mutation in one or more amino acids from among amino acids 13-27 in an AAV6 AAP polypeptide encoded by said AAP nucleotide sequence, or in a corresponding region of a non-AAV6 AAP polypeptide encoded by said AAP nucleotide sequence.

In some embodiments, said nucleic acid further comprises an AAV12 VP1 sequence, an AAV12 VP2 sequence, and an AAV6 VP3 sequence. In some embodiments, said AAP nucleotide sequence comprises K21L, C22L, L23W, M24D, M25L, and R27Q mutations in an AAV6 AAP polypeptide encoded by said AAP nucleotide sequence, or in a corresponding region of a non-AAV6 AAP polypeptide encoded by said AAP nucleotide sequence. In some embodiments, said AAP nucleotide sequence increases titer of an AAV comprising said nucleic acid as compared to a corresponding AAV comprising a comparable nucleic acid without said AAP nucleotide sequence. In some embodiments, said first and second AAP regions encode a functional AAP protein. In some embodiments, said first and second AAP regions are covalently linked.

In some aspects, the present disclosure provides a cell comprising the nucleic acid described above. In some aspects, the present disclosure provides a polypeptide expressed from the nucleic acid described above. In some aspects, the present disclosure provides a composition comprising the nucleic acid described above. In some aspects, the present disclosure provides a viral particle comprising the nucleic acid described above.

In some aspects, the present disclosure provides a system comprising a first nucleic acid that comprises an adeno-associated virus (AAV) nucleotide sequence comprising VP1, VP2, and VP3 sequences, wherein two of said VP1, VP2, and VP3 sequences are from a first AAV serotype, and one of said VP1, VP2, and VP3 sequence is from a second AAV serotype, and a second nucleic acid that comprises an assembly-activating protein (AAP) sequence that is heterologous to said first isolated non-naturally occurring nucleic acid sequence.

In some embodiments, said AAP sequence increases titer of an AAV comprising said first nucleic acid and said second nucleic acid as compared to a corresponding AAV comprising said first nucleic acid and not said second nucleic acid. In some embodiments, said AAP sequence is a wild-type AAV AAP sequence. In some embodiments, said AAP sequence is an AAV6 AAP sequence. In some embodiments, said first AAV serotype and said second AAV serotype are selected from AAV1, AAV2, AAV3, AAV4, AAV5, AAV6, AAV7, AAV8, AAV9, AAV10, AAV11, AAV12, or any combination thereof. In some embodiments, said first AAV serotype is AAV12 and said second AAV serotype is AAV6. In some embodiments, said VP1 and VP2 sequences are AAV12 sequences and said VP3 sequence is an AAV6 sequence. In some embodiments, after introduction into a plurality of cells, said nucleic acid confers an increased expression of a transgene as compared to a wild-type AAV nucleic acid

In some aspects, the present disclosure provides a system comprising a first nucleic acid that comprises an adeno-associated virus (AAV) nucleotide sequence comprising an AAV12 VP2 sequence, and a second nucleic acid that comprises an AAV6 assembly-activating protein (AAP) nucleotide sequence. In some embodiments, said nucleic acid further comprises an AAV12 VP1 sequence and an AAV6 VP3 sequence. In some embodiments, said AAP nucleotide sequence increases titer of an AAV comprising said first nucleic acid and said second nucleic acid as compared to a corresponding AAV comprising said first nucleic and not said second nucleic acid.

In some aspects, the present disclosure provides a cell comprising the system described above. In some aspects, the present disclosure provides a polypeptide expressed from the system described above. In some aspects, the present disclosure provides a composition comprising the system described above. In some aspects, the present disclosure provides a viral particle comprising the system described above.

In some aspects, the present disclosure provides a polynucleic acid sequence that encodes: in a first reading frame, an adeno-associated virus (AAV) VP1 polypeptide, an AAV VP2 polypeptide, and an AAV VP3 polypeptide, and in a second reading frame, a first AAV assembly-activating protein (AAP) polypeptide in a region encoding said VP2 polypeptide and a second AAV AAP polypeptide in a region encoding said VP3 polypeptide, wherein one of said VP1, VP2, and VP3 polypeptides are from a first AAV serotype, and one of said VP1, VP2, and VP3 polypeptides is from a second AAV serotype, and wherein said first AAP polypeptide comprises an amino acid substitution as compared to a wild-type AAV AAP polypeptide of the AAV serotype of the VP2 polypeptide or said second AAP polypeptide comprises an amino acid substitution as compared to a wild-type AAV AAP polypeptide of the AAV serotype of the VP3 polypeptide.

In some embodiments, said first and second AAP polypeptides increase titer of an AAV comprising said polynucleic acid sequence as compared to a corresponding AAV comprising a comparable polynucleic acid sequence without said first and second AAP polypeptides. In some embodiments, said at least one substitution mutation is in a helical region of said first AAP polypeptide or said second AAP polypeptide. In some embodiments, said at least one substitution mutation comprises one, two, three, four, five, six, seven, eight, nine, ten, eleven, twelve, or more substitution mutations. In some embodiments, said at least one substitution mutation comprises six substitution mutations. In some embodiments, said serotype of the VP2 polypeptide is an AAV6 serotype, and said at least one substitution mutation is within amino acids 13 to 27 of said AAP polypeptide. In some embodiments, said at least one substitution mutation is within amino acids 21 to 27 of said AAP polypeptide. In some embodiments, said at least one substitution mutation comprises K21L, C22L, L23W, M24D, M25L, and R27Q substitutions in said AAP polypeptide.

In some embodiments, said at least one amino acid substitution comprises a substitution at amino acid K53, C54, L55, M56, M57, and R59 of SEQ ID NO: 39, or any combination thereof, in said AAP polypeptide. In some embodiments, said at least one amino acid substitution comprises a substitution at amino acids K53, C54, L55, M56, M57, and R59 of SEQ ID NO: 39, in said AAP polypeptide. In some embodiments, said at least one amino acid substitution comprises a K53L, a C54L, a L55W, a M56D, a M57L, or a R59Q substitution in SEQ ID NO: 39, or any combination thereof, in said AAP polypeptide. In some embodiments, said at least one amino acid substitution comprises a K53L, a C54L, a L55W, a M56D, a M57L, and a R59Q substitution in SEQ ID NO: 39, in said AAP polypeptide.

In some embodiments, said first AAV serotype and said second AAV serotype are selected from AAV1, AAV2, AAV3, AAV4, AAV5, AAV6, AAV7, AAV8, AAV9, AAV10, AAV11, AAV12, or any combination thereof. In some embodiments, said first AAV serotype is AAV12 and said second AAV serotype is AAV6. In some embodiments, said VP1 and VP2 sequences are AAV12 sequences and said VP3 sequence is an AAV6 sequence. In some embodiments, after introduction into a plurality of cells, said polynucleic acid sequence confers an increased expression of a transgene as compared to a wild-type AAV nucleic acid.

In some aspects, the present disclosure provides a polynucleic acid sequence that comprises two or more adeno-associated virus (AAV) nucleic acid sequences, wherein said polynucleic acid sequence encodes, in a first reading frame, a VP2 polypeptide of a predetermined AAV serotype, and said polynucleic acid sequence encodes, in a second reading frame, an assembly-activating protein (AAP) polypeptide comprising a substitution mutation in one or more of amino acids 5-40 in said AAP polypeptide, wherein said substitution mutation is a coding mutation with respect to said predetermined AAV serotype.

In some embodiments, said polynucleic acid sequence comprises an AAV12 VP1 sequence, an AAV12 VP2 sequence, and an AAV6 VP3 sequence. In some embodiments, said predetermined AAV serotype is AAV6, and said substitution mutation comprises K21L, C22L, L23W, M24D, M25L, and R27Q mutations in said AAP polypeptide. In some embodiments, said polynucleic acid sequence increases titer of an AAV comprising said polynucleic acid sequence as compared to a corresponding AAV comprising a comparable polynucleic acid without said substitution mutation. In some embodiments, said first and second AAP polypeptides encode a functional AAP polypeptide. In some embodiments, said first and second AAP polypeptides are directly covalently linked.

In some aspects, the present disclosure provides a cell comprising the polynucleic acid sequence described above. In some aspects, the present disclosure provides a polypeptide expressed from the polynucleic acid sequence described above. In some aspects, the present disclosure provides a composition comprising the polynucleic acid sequence described above. In some aspects, the present disclosure provides a viral particle comprising the polynucleic acid sequence described above.

In some aspects, the present disclosure provides a system comprising a first polynucleic acid sequence that comprises three or more adeno-associated virus (AAV) nucleic acid sequences, wherein said first polynucleic acid sequence encodes a VP1 polypeptide, a VP2 polypeptide, and a VP3 polypeptide, wherein two of said VP1, VP2, and VP3 polypeptides are from a first AAV serotype, and one of said VP1, VP2, and VP3 polypeptides is from a second AAV serotype, and a second polynucleic acid sequence that encodes an assembly-activating protein (AAP) polypeptide that is heterologous to said first polynucleic acid sequence, wherein said first polynucleic acid sequence and second polynucleic acid sequence are not covalently linked.

In some embodiments, said AAP polypeptide increases titer of an AAV comprising said first polynucleic acid sequence and said second polynucleic acid sequence as compared to a corresponding AAV comprising said first polynucleic acid sequence and not said second polynucleic acid sequence. In some embodiments, In some embodiments, said AAP polypeptide is a wild-type AAV AAP polypeptide. In some embodiments, said AAP polypeptide is an AAV6 AAP polypeptide. In some embodiments, said first AAV serotype and said second AAV serotype are selected from AAV1, AAV2, AAV3, AAV4, AAV5, AAV6, AAV7, AAV8, AAV9, AAV10, AAV11, AAV12, or any combination thereof. In some embodiments, said second AAV serotype is AAV6. In some embodiments, said first polynucleic acid sequence comprises AAV12 VP1 and VP2 polynucleic acid sequences and an AAV6 VP3 polynucleic acid sequence. In some embodiments, after introduction into a plurality of cells, said first and second polynucleic acid sequences confer an increased expression of a transgene as compared to a wild-type AAV polynucleic acid.

In some aspects, the present disclosure provides a system comprising a first polynucleic acid sequence that comprise an adeno-associated virus (AAV) nucleic acid sequence, wherein said first polynucleic acid sequence encodes an AAV12 VP2 polypeptide, and a second polynucleic acid sequence that encodes an assembly-activating protein (AAP) polypeptide that is heterologous to said first polynucleic acid sequence, wherein said first polynucleic acid sequence and second polynucleic acid sequence are not covalently linked.

In some embodiments, said first polynucleic acid sequence further comprises an AAV12 VP1 sequence and an AAV6 VP3 sequence. In some embodiments, said AAP polypeptide increases titer of an AAV comprising said first polynucleic acid sequence and said second polynucleic acid sequence as compared to a corresponding AAV comprising said first polynucleic acid sequence and not said second polynucleic acid sequence.

In some aspects, the present disclosure provides a cell comprising the system as described above. In some aspects, the present disclosure provides a polypeptide expressed from the system as described above. In some aspects, the present disclosure provides a composition comprising the system as described above. In some aspects, the present disclosure provides a viral particle comprising the system as described above.

In some aspects, the present disclosure provides a polynucleic acid sequence encoding an adeno-associated virus (AAV) VP1 polypeptide, an AAV VP2 polypeptide, an AAV VP3 polypeptide, and an AAV AAP polypeptide, wherein two of said VP1, VP2, and VP3 polypeptides are from a first AAV serotype, and one of said VP1, VP2, and VP3 polypeptides is from a second AAV serotype, and wherein said AAP polypeptide comprises one or more substitution mutations as compared to a wild-type AAP polypeptide of said first AAV serotype or said second AAV serotype.

In some embodiments, said AAP polypeptide increases titer of an AAV comprising said polynucleic acid sequence as compared to a corresponding AAV comprising a comparable polynucleic acid sequence without said AAP polypeptide. In some embodiments, said one or more substitution mutations is in a helical region of said first AAP polypeptide or said second AAP polypeptide. In some embodiments, said one or more substitution mutations comprises one, two, three, four, five, six, seven, eight, nine, ten, eleven, twelve, or more substitution mutations. In some embodiments, said one or more substitution mutations comprises six substitution mutations. In some embodiments, said serotype of said VP2 polypeptide is an AAV6 serotype, and said one or more substitution mutations is within amino acids 13 to 27 of said AAP polypeptide. In some embodiments, said one or more substitution mutations is within amino acids 21 to 27 of said AAP polypeptide. In some embodiments, said serotype of said VP2 polypeptide is an AAV6 serotype, and said one or more substitution mutations comprises K21L, C22L, L23W, M24D, M25L, and R27Q substitutions in said AAP polypeptide.

In some embodiments, said at least one amino acid substitution comprises a substitution at amino acid K53, C54, L55, M56, M57, and R59 of SEQ ID NO: 39, or any combination thereof, in said AAP polypeptide. In some embodiments, said at least one amino acid substitution comprises a substitution at amino acids K53, C54, L55, M56, M57, and R59 of SEQ ID NO: 39, in said AAP polypeptide. In some embodiments, said at least one amino acid substitution comprises a K53L, a C54L, a L55W, a M56D, a M57L, or a R59Q substitution in SEQ ID NO: 39, or any combination thereof, in said AAP polypeptide. In some embodiments, said at least one amino acid substitution comprises a K53L, a C54L, a L55W, a M56D, a M57L, and a R59Q substitution in SEQ ID NO: 39, in said AAP polypeptide.

In some embodiments, said first AAV serotype and said second AAV serotype are selected from AAV1, AAV2, AAV3, AAV4, AAV5, AAV6, AAV7, AAV8, AAV9, AAV10, AAV11, AAV12, or any combination thereof.

In some embodiments, said first AAV serotype is AAV12 and said second AAV serotype is AAV6. In some embodiments, said VP1 and VP2 sequences are AAV12 sequences and said VP3 sequence is an AAV6 sequence. In some embodiments, after introduction into a plurality of cells, said polynucleic acid sequence confers an increased expression of a transgene as compared to a wild-type AAV nucleic acid.

In some aspects, the present disclosure provides a cell comprising the polynucleic acid sequence as described above. In some aspects, the present disclosure provides a polypeptide expressed from the polynucleic acid sequence as described above. In some aspects, the present disclosure provides a composition comprising the polynucleic acid sequence as described above. In some aspects, the present disclosure provides a viral particle comprising the polynucleic acid sequence as described above.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a schematic of six designs of AAV chimeras described herein and their sequences as compared to WT AAV6. The amino acid residues (amino acids 13-27 in WT AAV6 AAP and the corresponding amino acids in the chimera AAP*) in the box are involved in the stability and assembly activity of AAP proteins and certain key amino acid residues (amino acids 21-27 in WT AAV6 AAP and the corresponding amino acids in the chimera AAP*) in this region are noted with asterisks (*). The substituted amino acid residue or residues in the chimeras are underlined. *The amino acid numbers are noted with respect to WT AAV6 AAP sequences and one of ordinary skill in the art would readily understand the alignment of the WT AAV6 and chimera AAP sequences to recognize the corresponding amino acid numbers in AAP chimera sequences.

FIG. 1B depicts a summary table showing the comparison of the virus titer of six AAV chimeras with modified AAP sequences in GC/ml. Details of the chimera design are also noted. The amino acid numbers noted in Details of design the table are with respect to WT AAV6 AAP sequences and the one of ordinary skill in the art would readily understand the alignment of the WT AAV6 and chimera AAP sequences in FIG. 1A to recognize the corresponding amino acid numbers in AAP chimera sequences.

FIG. 2 depicts an example bar graph of virus titer data of WT AAV6, chimeras 6, 6.1, 6.2, 6.3, 6.4, 6.5, and 6.6 in GC/mL.

FIG. 3 depicts a bar graph of luminescence (RLU) on day 5 post transduction of T-cells with WT AAV6, chimera 6, 6.1, or 6.3 (CMV NanoLuc virus) at MOI of 1e4 GC/mL, 1e5 GC/mL, or 1e6 GC/mL.

FIG. 4 depicts a bar graph of virus titer data of WT AAV6, chimera 6, and chimera 6 produced in the presence of Met or Leu versions of WT AAV6 AAP in GC/mL. Met and Leu versions of WT-AAV6 AAP only differ in their start codon.

FIG. 5 depicts an example of bar graph of luminescence (RLU) on day 5 post transduction of T-cells with WT AAV6, chimera 6, and chimera 6 produced in the presence of Met or Leu versions of WT AAV6 AAP (CMV NanoLuc virus) at MOI of 1e4 GC/mL. Met and Leu versions of WT-AAV6 AAP only differ in their start codon.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

All publications, patents, and patent applications mentioned in this specification are herein incorporated by reference in their entirety for all purposes, to the same extent as if each individual publication, patent, or patent application was specifically and individually indicated to be incorporated by reference. For example, all publications and patents mentioned herein are incorporated herein by reference in their entirety for the purpose of describing and disclosing the kits, compositions, and methodologies that are described in the publications, which might be used in connection with the methods, kits, and compositions described herein. The documents discussed herein are provided solely for their disclosure prior to the filing date of the present application. Nothing herein is to be construed as an admission that the inventors described herein are not entitled to antedate such disclosure by virtue of prior invention or for any other reason.

### Definitions

To facilitate an understanding of the present disclosure, a number of terms and phrases are defined below.

The terminology used herein is for the purpose of describing particular cases only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

It is understood that terms such as “comprises,” “comprised,” “comprising,” and the like have the meaning attributed to it in U.S. Patent law; i.e., they mean “includes,” “included,” “including,” and the like and are intended to be inclusive or open ended and does not exclude additional, unrecited elements or method steps; and that terms such as “consisting essentially of” and “consists essentially of” have the meaning ascribed to them in U.S. Patent law; i.e., they allow for elements not explicitly recited, but exclude elements that are found in the prior art or that affect a basic or novel characteristic of the invention.

The term “and/or” as used in a phrase such as “A and/or B” herein includes both A and B; A or B; A (alone); and B (alone). Likewise, the term “and/or” as used in a phrase such as “A, B, and/or C” encompass each of the following embodiments: A, B, and C; A, B, or C; A or B; A or C; B or C; A and B; A and C; B and C; A (alone); B (alone); and C (alone).

The term “about” or “approximately” means within an acceptable error range for the particular value as determined by one of ordinary skill in the art, which will depend in part on how the value is measured or determined, i.e., the limitations of the measurement system. For example, “about” and its grammatical equivalents in relation to a reference numerical value and its grammatical equivalents as used herein can include a range of values plus or minus 10% from that value. Or for example, the amount “about 10” can include amounts from 9 to 11. The term “about” in relation to a reference numerical value can also include a range of values plus or minus 10%, 9%, 8%, 7%, 6%, 5%, 4%, 3%, 2%, or 1% from that value.

The term “adeno-associated virus” or “AAV,” refers to an adeno-associated virus of any of the known serotypes, including e.g., AAV1, AAV2, AAV3, AAV4, AAV5, AAV6, AAV7, AAV8, AAV9, AAV10, AAV11, or AAV12, scAAV (self-complementary AAV), rh10, chimeric, or hybrid AAV, or any combination, derivative, or variant thereof. AAV is a small non-enveloped single-stranded DNA virus. They are non-pathogenic parvoviruses and can require helper viruses, such as adenovirus, herpes simplex virus, vaccinia virus, and CMV, for replication. Wild-type (WT) AAV is common in the general population, and is not associated with any known pathologies. AAV, as used herein, includes avian AAV, bovine AAV, canine AAV, equine AAV, primate AAV, non-primate AAV, and ovine AAV, wherein primate AAV refers to AAV that infects primates, and wherein non-primate AAV refers to AAV that infects non-primate animals, such as avian AAV that infects avian animals. In some cases, the WT AAV contains rep and cap genes, wherein the rep gene is required for viral replication and the cap gene is required for the synthesis of capsid proteins. The abbreviation “rAAV” refers to recombinant adeno-associated virus, also referred to as a recombinant AAV.

The term “hybrid AAV” as used herein refers to an AAV comprising a capsid protein of one AAV serotype and genomic material from another AAV serotype.

The term “chimeric AAV” as used herein refers to an AAV that comprises genetic and/or protein sequences derived from two or more AAV serotypes, and can include mutations made to the genetic sequences of those two or more AAV serotypes. An exemplary chimeric AAV can comprise a chimeric AAV capsid, for example, a capsid protein with one or more regions of amino acids derived from two or more AAV serotypes.

The term “AAV variant” as used herein refers to an AAV comprising one or more amino acid mutations in its genome or proteins as compared to its parental AAV, e.g., one or more amino acid mutations in its capsid protein as compared to its parental AAV.

The term “viral vector” refers to a gene transfer vector or a gene delivery system derived from a virus. Such vector can be constructed using recombinant techniques known in the art. In some aspects, the virus for deriving such vector is selected from adeno-associated virus (AAV), helper-dependent adenovirus, hybrid adenovirus, Epstein-Bar virus, retrovirus, lentivirus, herpes simplex virus, hemmaglutinating virus of Japan (HVJ), Moloney murine leukemia virus, poxvirus, and HIV-based virus.

The term “AAV virion” or “AAV particle,” as used herein refers to a virus particle comprising a capsid comprising at least one AAV capsid protein that encapsidates an AAV vector as described herein, wherein the vector can further comprise a heterologous polynucleotide sequence or a transgene in some embodiments.

The term “viral vector” refers to a gene transfer vector or a gene delivery system derived from a virus. Such vector can be constructed using recombinant techniques known in the art. In some aspects, the virus for deriving such vector is selected from adeno-associated virus (AAV), helper-dependent adenovirus, hybrid adenovirus, Epstein-Bar virus, retrovirus, lentivirus, herpes simplex virus, hemmaglutinating virus of Japan (HVJ), Moloney murine leukemia virus, poxvirus, and HIV-based virus.

The term “engineered cell” and its grammatical equivalents as used herein refers to a cell comprising at least one alterations of a nucleic acid within the cell's genome or comprising at least one exogenous nucleic acid or protein. Alterations include additions, deletions, and/or substitutions within a nucleic acid sequence. As such, engineered cells, include cells that contain an added, deleted, and/or altered gene.

The term “mutation” and its grammatical equivalents as used herein includes a substitution, deletion, and/or insertion of a nucleotide of a nucleic acid sequence or a substitution, deletion, and/or insertion of an amino acid in a polypeptide sequence. A mutation can be a conservative mutation or replacement. For example, 20 naturally occurring amino acids can share similar characteristics. Aliphatic amino acids can be: glycine, alanine, valine, leucine, or isoleucine. Hydroxyl or sulfur/selenium-containing amino acids can be: serine, cysteine, selenocysteine, threonine, or methionine. A cyclic amino acid can be proline. An aromatic amino acid can be phenylalanine, tyrosine, or tryptophan. A basic amino acid can be histidine, lysine, or arginine. An acidic amino acid can be aspartate, glutamate, asparagine, or glutamine. A conservative mutation can be: serine to glycine, serine to alanine, serine to serine, serine to threonine, or serine to proline; arginine to asparagine, arginine to lysine, arginine to glutamine, arginine to arginine, or arginine to histidine; leucine to phenylalanine, leucine to isoleucine, leucine to valine, leucine to leucine, or leucine to methionine; proline to glycine, proline to alanine, proline to serine, proline to threonine, or proline to proline; threonine to glycine, threonine to alanine, threonine to serine, threonine to threonine, or threonine to proline; alanine to glycine, alanine to threonine, alanine to proline, alanine to alanine, or alanine to serine; valine to methionine, valine to phenylalanine, valine to isoleucine, valine to leucine, or valine to valine; glycine to alanine, glycine to threonine, glycine to proline, glycine to serine, or glycine to glycine; isoleucine to phenylalanine, isoleucine to isoleucine, isoleucine to valine, isoleucine to leucine, or isoleucine to methionine; phenylalanine to tryptophan, phenylalanine to phenylalanine, or phenylalanine to tyrosine; tyrosine to tryptophan, tyrosine to phenylalanine, or tyrosine to tyrosine; cysteine to serine, cysteine to threonine, or cysteine to cysteine; histidine to asparagine, histidine to lysine, histidine to glutamine, histidine to arginine, or histidine to histidine; glutamine to glutamic acid, glutamine to asparagine, glutamine to aspartic acid, or glutamine to glutamine; asparagine to glutamic acid, asparagine to asparagine, asparagine to aspartic acid, or asparagine to glutamine; lysine to asparagine, lysine to lysine, lysine to glutamine, lysine to arginine, or lysine to histidine; aspartic acid to glutamic acid, aspartic acid to asparagine, aspartic acid to aspartic acid, or aspartic acid to glutamine; glutamine to glutamine, glutamine to asparagine, glutamine to aspartic acid, glutamine to glutamine; methionine to phenylalanine, methionine to isoleucine, methionine to valine, methionine to leucine, or methionine to methionine; tryptophan to tryptophan, tryptophan to phenylalanine, or tryptophan to tyrosine.

The term “heterologous” and its grammatical equivalents as used herein refers to being different, changed, or altered from the original nucleotide or peptide sequence. For example, a chimeric AAV of two different AAV serotypes can have a nucleotide sequence that is different from or heterologous to both serotypes.

The term “transgene” and its grammatical equivalents as used herein refers to a gene or genetic material that is transferred into a cell ex vivo, in vivo, or in vitro. For example, a transgene can be a stretch or segment of DNA containing a gene that is introduced into a cell ex vivo, in vivo, or in vitro. When a transgene is transferred into a cell in an organism in vivo, the organism is then referred to as a transgenic organism. In some embodiments, the transgene retains its ability to produce an RNA and/or functional proteins An exemplary transgene described herein encodes for an engineered T-cell receptor. A transgene can be a receptor. A transgene can comprise recombination arms. A transgene can comprise engineered sites.

The term “antigen” and its grammatical equivalents as used herein refers to a molecule that contains one or more epitopes capable of being bound by one or more receptors, antibodies (including functional fragments or variants thereof) or other antigen binding moieties. For example, an antigen can stimulate a host's immune system to make a cellular antigen-specific immune response when the antigen is presented, or a humoral antibody response. An antigen can also have the ability to elicit a cellular and/or humoral response by itself or when present in combination with another molecule. For example, a tumor cell antigen can be recognized by a TCR.

The term “epitope” and its grammatical equivalents as used herein refers to a part of an antigen that can be recognized by antibodies (including functional fragments or variants thereof), B-cells (through the B cell receptor), T-cells (through the T cell receptor (TCR)), cell surface receptors, or other epitope binding moieties or receptors (e.g., a chimeric antigen receptor (CAR)). For example, an epitope can be a cancer epitope that is recognized by a TCR. Multiple epitopes within an antigen can also be recognized. The epitope can also be mutated.

The term “recombination” and its grammatical equivalents as used herein refers to a process of exchange of genetic information between two polynucleic acids. For the purposes of this disclosure, “homologous recombination” or “HR” refers to a specialized form of such genetic exchange that can take place, for example, during repair of double-strand breaks. This process requires nucleotide sequence homology, for example, using a donor molecule to template repair of a target molecule (e.g., a molecule that experienced the double-strand break), and is sometimes known as non-crossover gene conversion or short tract gene conversion. Such transfer can also involve mismatch correction of heteroduplex DNA that forms between the broken target and the donor, and/or synthesis-dependent strand annealing, in which the donor can be used to resynthesize genetic information that can become part of the target, and/or related processes. Such specialized HR can often result in an alteration of the sequence of the target molecule such that part or all of the sequence of the donor polynucleotide can be incorporated into the target polynucleotide. The terms “recombination arms” and “homology arms” are used interchangeably herein.

The term “non-human animal” and its grammatical equivalents as used herein includes all animal species other than humans, including non-human mammals, which can be a native animal or a genetically modified non-human animal.

The terms “nucleic acid,” “polynucleotide,” “polynucleic acid,” and “oligonucleotide” and their grammatical equivalents are used interchangeably herein and refer to a deoxyribonucleotide or ribonucleotide polymer, in linear or circular conformation, and in either single- or double-stranded form. For the purposes of the present disclosure, these terms should not to be construed as limiting with respect to length. The terms also encompass nucleic acids comprising analogues of natural nucleotides, as well as nucleotides that are modified in the base, sugar and/or phosphate moieties (e.g., phosphorothioate backbones). Modifications of the terms can also encompass demethylation, addition of CpG methylation, removal of bacterial methylation, and/or addition of mammalian methylation. In general, an analogue of a particular nucleotide can have the same base-pairing specificity, i.e., an analogue of A can base-pair with T.

The term “autologous” and its grammatical equivalents as used herein refers to cells or tissues are obtained from and administered to the same subject. For example, a sample (e.g., cells) can be removed, processed, and given back to the same subject at a later time. An autologous process is distinguished from an allogenic process where the donor and the recipient are different subjects.

The term “allogenic” and its grammatical equivalents as used herein refers to cells or tissues are obtained from one subject and administered to a different subject of the same species. For example, a sample (e.g., cells) can be removed, processed, and given back to a different subject of the same species at a later time.

The terms “cancer” and “tumor” are used interchangeably herein and refer to a hyperproliferation of cells whose unique trait—loss of normal controls—results in unregulated growth, lack of differentiation, local tissue invasion, and metastasis. With respect to the methods described herein, the cancer can be any cancer, including, but not limited to, acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer.

### Overview

Disclosed herein are modified adeno-associated viruses (AAV) with optionally one or more of superior viral titer and infectivity compared to unmodified AAV, compositions comprising said viruses, methods for producing or using the same, and methods of using the same in the treatment of conditions, for instance cancer. In some embodiments, the viruses described herein comprise a modified AAP sequence that can confer an increased viral titer as compared to a corresponding virus without the modified AAP sequence. In some embodiments, chimeric AAV vectors or mutated chimeric AAV vectors are used for delivering an exogenous cellular receptor in a way that improves physiologic and immunologic potency of an engineered cell (e.g., an immune cell). In some embodiments, modified AAV vectors are useful to treat various indications, including, for example, cancer (e.g., metastatic cancer). In some embodiments, AAV vector-modified cells comprise a genomic disruption of at least one gene.

### Modified Adeno-Associated Viral (AAV) Vectors

### Overview

Adeno-associated viral (AAV) vectors can be utilized to introduce a transgene into a cell. In some embodiments, said AAV vector is a chimeric AAV vector. In some embodiments, said chimeric AAV vector has superior viral infectivity as compared to a wild-type or non-chimeric AAV vector, and lower viral titer as compared to the wild-type or non-chimeric AAV. The present disclosure provides, inter alia, nucleic acids encoding modified AAP sequences that increase viral titer as compared to AAV without said modified AAP sequences, or compared to a comparable chimeric AAV without said modified AAP sequences. In some embodiments, the modified AAP sequence is provided as part of a nucleic acid molecule encoding the capsid proteins VP1, VP2, and VP3. In some embodiments, the modified AAP sequence is provided in trans as a separate nucleic acid molecule than the nucleic acid molecule encoding the capsid proteins VP1, VP2, and VP3 (e.g., VP1, VP2, and VP3 polypeptides are encoded by a polynucleic acid molecule that is not covalently linked to a polynucleic acid molecule encoding a modified AAP polypeptide).

The AAV genome carries two viral genes: rep and cap. The virus utilizes two promoters and alternative splicing to generate four proteins necessary for replication (Rep78, Rep68, Rep52, and Rep40), while a third promoter generates the transcript for three structural viral capsid proteins 1, 2, and 3 (VP1, VP2, and VP3), through a combination of alternate splicing and alternate translation start codons. As used herein, “VP1u” refers to the unique sequence of VP1 (i.e. the sequence that does not overlap with VP2 and/or VP3). The three capsid proteins share the same C-terminal 533 amino acids, while VP1 and VP2 contain additional N-terminal sequences of 202 and 65 amino acids, respectively. The AAV virion can contain a total of 60 copies of VP1, VP2, and VP3 at a 1:1:20 ratio, arranged in a T=1 icosahedral symmetry. In some cases, a Rep protein (e.g., Rep78, Rep68, Rep52, or Rep40) or a capsid protein can be modified and utilized in the disclosed compositions and methods. In some cases, the capsid is comprised of three VPs: VP1, VP2, and VP3. The VP1 protein contains the entire VP2 sequence in addition to a unique 137-amino-acid N-terminal region (VP1u), while the VP2 protein contains the entire VP3 sequence in addition to an 65-amino-acid N-terminal region (VP1/2 common region). In some embodiments, an AAV provided herein comprises an assembly-activating protein (AAP). In certain embodiments, the AAP promotes capsid assembly. In some cases, an AAV comprises an AAP polypeptide modified to enhance AAV capsid structure and function, for example by improving capsid assembly. In some embodiments, for example, a modified Rep protein or capsid protein provides improved packaging efficiency, yield, infectivity, transduction efficiency, or transfection efficiency. In some embodiments, said AAV has a capsid diameter of about 26 nm. In some embodiments, said capsid diameter is from about 20 nm to about 50 nm in some cases.

At the cellular level, AAV can undergo 5 steps prior to achieving gene expression: 1) binding or attachment to cellular surface receptors, 2) endocytosis, 3) trafficking to the nucleus, 4) uncoating of the virus to release the genome, and 5) conversion of the genome from single-stranded to double-stranded DNA as a template for transcription in the nucleus. The cumulative efficiency with which AAV can successfully execute each individual step can determine the overall transduction efficiency. Rate limiting steps in AAV transduction can include the absence or low abundance of required cellular surface receptors for viral attachment and internalization, inefficient endosomal escape leading to lysosomal degradation, and slow conversion of single-stranded to double-stranded DNA template. Therefore, vectors with modifications to the genome and/or the capsids can be designed to facilitate more efficient or more specific transduction of cells or tissues for gene therapy.

In some cases, a host cell can contain sequences which drive expression of a novel AAV capsid protein (or a capsid protein comprising a fragment thereof) in the host cell and rep sequences of the same source as the source of the AAV ITRs, or a cross-complementing source. The AAV cap and rep sequences can be independently obtained from an AAV source as described above and can be introduced into the host cell in any manner known to one of ordinary skill in the art as described above. Additionally, when pseudotyping an AAV vector, the sequences encoding each of the Rep proteins can be supplied by different AAV sources (e.g., AAV1, AAV2, AAV3, AAV4, AAV5, AAV6, AAV7, AAV8, AAV9, AAV10, AAV11, AAV12). In some cases, a host cell stably contains the capsid protein under the control of a suitable promoter. In some cases, a capsid protein can be expressed under the control of an inducible promoter. In another embodiment, a nucleic acid encoding a capsid protein can be supplied to the host cell in trans from a nucleic acid encoding a rep sequence. Likewise, an AAP nucleic acid sequence can be supplied to the host cell in trans from the nucleic acid encoding a capsid protein and/or from the nucleic acid encoding a rep sequence. When delivered to the host cell in trans, a protein can be delivered via a plasmid which contains the sequences necessary to direct expression of the selected protein in the host cell. In some cases, when delivered to a host cell in trans, a plasmid carrying a protein also carries other sequences required for packaging the AAV, e.g., the rep sequences. In some cases, rep, cap, and AAP sequences can be transfected into a host cell on a single nucleic acid molecule and exist stably in the cell as an episome. In another embodiment, the rep, cap, and AAP sequences are stably integrated into the chromosome of the cell. Another embodiment has the rep, cap, and AAP sequences are transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence including the AAP sequence.

In some cases, novel AAV amino acid sequences, peptides and proteins can be expressed from AAV nucleic acid sequences described herein. Additionally, these amino acid sequences, peptides and proteins can be generated by other methods known in the art, including, e.g., by chemical synthesis, by other synthetic techniques, or by other methods. The sequences of any of the AAV capsids provided herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, N.Y.). Alternatively, peptides can also be synthesized by the well-known solid phase peptide synthesis methods (Merrifield, J. Am. Chem. Soc., 85:2149 (1962); Stewart and Young, Solid Phase Peptide Synthesis (Freeman, San Francisco, 1969) pp. 27-62). The sequences and proteins described herein can be produced by any suitable means, including recombinant production, chemical synthesis, or other synthetic means. Such production methods are within the knowledge of those of skill in the art.

In some cases, sequences can encode an AAV capsid or engineered AAV vector described herein. In another embodiment, vectors can contain, at a minimum, sequences encoding an AAV Rep protein or a fragment thereof. Optionally, vectors can contain AAV Cap, Rep, and AAP proteins. In vectors in which AAV rep and cap (including AAP) sequences are provided, the AAV rep and AAV cap sequences can originate from an AAV of the same clade. Alternatively, provided herein can be vectors in which a rep sequences are from an AAV source which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV source to form a chimeric AAV vector. Optionally, vectors can be vectors packaged in an AAV capsid. These vectors and other vectors described herein can further contain a transgene comprising a selected transgene which is flanked by AAV 5′ ITR and AAV 3′ ITR.

In some embodiments, the AAV viral vector is isogenic. In some embodiments, the AAV viral vector is integrated into a portion of a genome with known SNPs. In some embodiments, the AAV vector cannot be integrated into a portion of a genome with known SNPs. For example, an AAV can be designed to be isogenic or homologous to a subject's own genomic DNA. In some embodiments, an isogenic vector improves the efficiency of homologous recombination (HR). In some embodiments, a guide RNA (gRNA) is designed so that it does not target a region of the genome with known SNPs in order to improve the expression of an integrated transgene. The frequency of SNPs at immune checkpoint genes, such as PD-1, CISH, and CTLA-4, are determined. In some embodiments, the frequency of SNPs at an endogenous TCR gene are be determined.

### Capsid Modifications and Chimeras

In some embodiments, an AAV viral capsid is modified. In some embodiments, the modification comprises a modification to at least 1, 2, or 3 capsid genes (e.g., VP1, VP2, or VP3). In some embodiments, VP1 is modified, VP2 is modified, VP3 is modified, VP1 and VP2 are modified, VP1 and VP3 are modified, VP2 and VP3 are modified, or VP1, VP2, and VP3 are modified, or any combination thereof.

In some embodiments, said modification comprises at least one amino acid modification (e.g., substitution, deletion, or addition), compared to the WT AAV capsid protein of the relevant serotype. A modification can be of any AAV serotype. In some embodiments, a modification is of a wild-type (WT) AAV6. A modification can include modifying a combination of capsid components. For example, a mosaic capsid AAV is a virion that can be composed of a mixture of viral capsid proteins from different serotypes. The capsid proteins can be provided by complementation with separate plasmids that are mixed at various ratios. During viral assembly, the different serotype capsid proteins can be mixed in each virion, at subunit ratios stoichiometrically reflecting the ratios of the complementing plasmids. A mosaic capsid can confer increased binding efficacy to certain cell types or improved performance as compared to an unmodified capsid.

In some embodiments, an AAV comprises a mutation in at least one capsid protein (e.g., at least one of VP1, VP2, and VP3). Thus, at least one of VP1, VP2, and VP3 has at least one amino acid substitution compared to WT AAV capsid protein. In some cases, a mutation can occur in VP1 and VP2, in VP1 and VP3, in VP2 and VP3, or in VP1, VP2, and VP3. In some cases, a VP can be removed. For example, in some embodiments a mutant AAV does not comprise at least one of VP1, VP2, or VP3.

In some embodiments, at least one of VP1, VP2, and VP3 has from one to about 15 amino acid substitutions compared to WT AAV VP1, VP2, and VP3, e.g., from about one to about 3, from about 3 to about 6, from about 6 to about 9, from about 9 to about 12, or from about 12 to about 15 amino acid substitutions compared to WT AAV VP1, VP2, and VP3. In some cases, a mutant AAV virion can have from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or up to about 100 mutations in at least a portion of an AAV sequence, such as a capsid or AAP sequence. A mutation in a capsid sequence can be within anyone of VP1, VP2, VP3, or combinations thereof. In some cases, a mutant AAV variant can have one mutation in a capsid sequence. In some cases, a mutant AAV variant can have two mutations in a capsid sequence. In some cases, a mutant AAV variant can have three mutations in a capsid sequence. Alternatively, a subject mutant AAV virion comprises one or more amino acid deletions and/or insertions in at least one capsid protein relative to WT capsid or AAP protein. In some embodiments, a subject mutant AAV virion comprises one or more amino acid substitutions and/or deletions and/or insertions in a capsid protein relative to a WT capsid protein. In some cases, a mutation can be a point mutation. In some cases, at least a portion of an AAV can be mutated. For example, a capsid of an AAV can have a mutation such as a point mutation, missense mutation, nonsense mutation, insertion, deletion, duplication, frameshift, or repeat expansion.

In some embodiments, the AAV is chimeric. In some embodiments, said chimeric AAV comprises a chimeric capsid. Chimeric capsid modifications include, but are not limited to, the use of naturally existing AAV serotypes as templates, which can involve AAV capsid sequences lacking a certain function being co-transfected with DNA sequences from another capsid. In some embodiments, said chimera includes at least one Cap polypeptide from an AAV serotype chosen from: AAV1, AAV2, AAV3, AAV4, AAV5, AAV6, AAV7, AAV8, AAV9, AAV10, AAV11, and AAV12. In some embodiments, said chimeric AAVs comprise a polypeptide encoding a VP1 from an AAV serotype chosen from the group consisting of: AAV1, AAV2, AAV3, AAV4, AAV5, AAV6, AAV7, AAV8, AAV9, AAV10, AAV11, and AAV12; a polypeptide comprising a VP2 from an AAV serotype chosen from: AAV1, AAV2, AAV3, AAV4, AAV5, AAV6, AAV7, AAV8, AAV9, AAV10, AAV11, and AAV12; and a VP1 from an AAV serotype chosen from the group consisting of: AAV1, AAV2, AAV3, AAV4, AAV5, AAV6, AAV7, AAV8, AAV9, AAV10, AAV11, and AAV12; wherein at least two of said VP1, VP2 and VP3 are from different AAV serotypes. In some embodiments, said chimeric capsid has an insertion of a foreign protein sequence, either from another WT AAV sequence or an unrelated protein, into the open reading frame of the capsid gene.

In some embodiments, said chimera comprises capsid proteins from: AAV4 and AAV6, AAV5 and AAV6, AAV11 and AAV6, AAV12 and AAV6, or any combination thereof. In some embodiments said chimera comprises a capsid protein from a first AAV serotype and a capsid protein from a second AAV serotype. In some embodiments, said first AAV serotype is AAV4 and said second serotype is AAV6. In some embodiments, said first AAV serotype is AAV5 and said second AAV serotype is AAV6. In some embodiments, said first AAV serotype is AAV11 and said second AAV serotype is AAV6. In some embodiments, said first AAV serotype is AAV12 and said second AAV serotype is AAV6.

Table 1 provides exemplary chimeric AAV capsid nucleic acid and amino acid sequences. Exemplary WT AAV capsid nucleic acid and amino acid sequences are provided in Table 2.

In some embodiments, the chimera comprises a capsid encoded by a nucleic acid sequence in Table 1. In some embodiments, the chimera comprises a capsid comprising an amino acid sequence in Table 1. In some embodiments, the chimera comprises a capsid protein encoded by a nucleic acid sequence that shares at least 80%, 85%, 90%, 95%, 96%, 97%, 98%, 99% or 100% identity with SEQ ID NOs: 51-65. In some embodiments, the chimera comprises a capsid protein that comprises an amino acid sequence that shares at least 80%, 85%, 90%, 95%, 96%, 97%, 98%, 99% or 100% identity with SEQ ID NOs: 44-50. In some embodiments, the chimera comprises a capsid protein encoded by a nucleic acid sequence that shares at least 99% or 100% identity with SEQ ID NOs: 51-65. In some embodiments, the chimera comprises a capsid protein that comprises an amino acid sequence that shares at least 99% or 100% identity with SEQ ID NOs: 44-50.

TABLE 1
Exemplary chimeric AAV capsid nucleic acid and amino acid sequences

| | SEQ | | SEQ | |
|---|---|---|---|---|
| Name | ID NO. | Amino Acid Sequence | ID NO. | Nucleic Acid Sequence |
| Chimera 2 | 44 | MSFVDHPPDWLEEVGEGL | 51 | atgtcttttgttgatcaccctccagattggtt |
| AAV5VP1u- | | REFLGLEAGPPKPKPNQQ | | ggaagaagttggtgaaggtcttcgcgag |
| AAV6VP2/3 | | HQDQARGLVLPGYNYLG | | tttttgggccttgaagcgggcccaccgaa |
| | | PGNGLDRGEPVNRADEVA | | accaaaacccaatcagcagcatcaagatc |
| | | REHDISYNEQLEAGDNPY | | aagcccgtggtcttgtgctgcctggttata |
| | | LKYNHADAEFQEKLADD | | actatctcggacccggaaacggtctcgat |
| | | TSFGGNLGKAVFQAKKRV | | cgaggagagcctgtcaacagggcagac |
| | | LEPFGLVEEGAKTAPGKK | | gaggtcgcgcgagagcacgacatctcgt |
| | | RPVEQSPQEPDSSSGIGKT | | acaacgagcagcttgaggcgggagaca |
| | | GQQPAKKRLNFGQTGDSE | | acccctacctcaagtacaaccacgcggac |
| | | SVPDPQPLGEPPATPAAV | | gccgagtttcaggagaagctcgccgacg |
| | | GPTTMASGGGAPMADNN | | acacatccttcgggggaaacctcggaaa |
| | | EGADGVGNASGNWHCDS | | ggcagtctttcaggccaagaaaagggttc |
| | | TWLGDRVITTSTRTWALP | | tcgaaccttttggcctggttgaagagggtg |
| | | TYNNHLYKQISSASTGAS | | ctaagacggctcctggaaagaaacgtcc |
| | | NDNHYFGYSTPWGYFDF | | ggtagagcagtcgccacaagagccaga |
| | | NRFHCHFSPRDWQRLINN | | ctcctcctcgggcattggcaagacaggcc |
| | | NWGFRPKRLNFKLFNIQV | | agcagcccgctaaaaagagactcaatttt |
| | | KEVTTNDGVTTIANNLTS | | ggtcagactggcgactcagagtcagtccc |
| | | TVQVFSDSEYQLPYVLGS | | cgacccacaacctctcggagaacctcca |
| | | AHQGCLPPFPADVFMIPQ | | gcaacccccgctgctgtgggacctactac |
| | | YGYLTLNNGSQAVGRSSF | | aatggcttcaggcggtggcgcaccaatg |
| | | YCLEYFPSQMLRTGNNFT | | gcagacaataacgaaggcgccgacgga |
| | | FSYTFEDVPFHSSYAHSQS | | gtgggtaatgcctcaggaaattggcattgc |
| | | LDRLMNPLIDQYLYYLNR | | gattccacatggctgggcgacagagtcat |
| | | TQNQSGSAQNKDLLFSRG | | caccaccagcacccgaacatgggccttg |
| | | SPAGMSVQPKNWLPGPC | | cccacctataacaaccacctctacaagca |
| | | YRQQRVSKTKTDNNNSNF | | aatctccagtgcttcaacgggggccagca |
| | | TWTGASKYNLNGRESIINP | | acgacaaccactacttcggctacagcacc |
| | | GTAMASHKDDKDKFFPM | | ccctgggggtattttgatttcaacagattcc |
| | | SGVMIFGKESAGASNTAL | | actgccatttctcaccacgtgactggcagc |
| | | DNVMITDEEEIKATNPVA | | gactcatcaacaacaattggggattccgg |
| | | TERFGTVAVNLQSSSTDP | | cccaagagactcaacttcaagctcttcaac |
| | | ATGDVHVMGALPGMVW | | atccaagtcaaggaggtcacgacgaatg |
| | | QDRDVYLQGPIWAKIPHT | | atggcgtcacgaccatcgctaataacctta |
| | | DGHFHPSPLMGGFGLKHP | | ccagcacggttcaagtcttctcggactcg |
| | | PPQILIKNTPVPANPPAEFS | | gagtaccagttgccgtacgtcctcggctct |
| | | ATKFASFITQYSTGQVSVE | | gcgcaccagggctgcctccctccgttccc |
| | | IEWELQKENSKRWNPEVQ | | ggcggacgtgttcatgattccgcagtacg |
| | | YTSNYAKSANVDFTVDN | | gctacctaacgctcaacaatggcagccag |
| | | NGLYTEPRPIGTRYLTRPL | | gcagtgggacggtcatccttttactgcctg |
| | | | | gaatatttcccatcgcagatgctgagaacg |
| | | | | ggcaataactttaccttcagctacaccttcg |
| | | | | aggacgtgcctttccacagcagctacgcg |
| | | | | cacagccagagcctggaccggctgatga |
| | | | | atcctctcatcgaccagtacctgtattacct |
| | | | | gaacagaactcagaatcagtccggaagt |
| | | | | gcccaaaacaaggacttgctgtttagccg |
| | | | | ggggtctccagctggcatgtctgttcagcc |
| | | | | caaaaactggctacctggaccctgttacc |
| | | | | ggcagcagcgcgtttctaaaacaaaaaca |
| | | | | gacaacaacaacagcaactttacctggac |
| | | | | tggtgcttcaaaatataaccttaatgggcgt |
| | | | | gaatctataatcaaccctggcactgctatg |
| | | | | gcctcacacaaagacgacaaagacaagt |
| | | | | tctttcccatgagcggtgtcatgatttttgga |
| | | | | aaggagagcgccggagcttcaaacactg |
| | | | | cattggacaatgtcatgatcacagacgaa |
| | | | | gaggaaatcaaagccactaaccccgtgg |
| | | | | ccaccgaaagatttgggactgtggcagtc |
| | | | | aatctccagagcagcagcacagaccctg |
| | | | | cgaccggagatgtgcatgttatgggagcc |
| | | | | ttacctggaatggtgtggcaagacagaga |
| | | | | cgtatacctgcagggtcctatttgggccaa |
| | | | | aattcctcacacggatggacactttcaccc |
| | | | | gtctcctctcatgggcggctttggacttaa |
| | | | | gcacccgcctcctcagatcctcatcaaaa |
| | | | | acacgcctgttcctgcgaatcctccggca |
| | | | | gagttttcggctacaaagtttgcttcattcat |
| | | | | cacccagtattccacaggacaagtgagc |
| | | | | gtggagattgaatgggagctgcagaaag |
| | | | | aaaacagcaaacgctggaatcccgaagt |
| | | | | gcagtatacatctaactatgcaaaatctgc |
| | | | | caacgttgatttcactgtggacaacaatgg |
| | | | | actttatactgagcctcgccccattggcac |
| | | | | ccgttacctcacccgtcccctgtaa |
| Chimera 3 | 45 | MTDGYLPDWLEDNLSEG | 52 | atgactgacggttaccttccagattggcta |
| rAAV4P1/2- | | VREWWALQPGAPKPKAN | | gaggacaacctctctgaaggcgttcgaga |
| AAV6VP3 | | QQHQDNARGLVLPGYKY | | gtggtgggcgctgcaacctggagcccct |
| | | LGPGNGLDKGEPVNAAD | | aaacccaaggcaaatcaacaacatcagg |
| | | AAALEHDKAYDQQLKAG | | acaacgctcggggtcttgtgcttccgggtt |
| | | DNPYLKYNHADAEFQQR | | acaaatacctcggacccggcaacggact |
| | | LQGDTSFGGNLGRAVFQA | | cgacaagggggaacccgtcaacgcagc |
| | | KKRVLEPLGLVEQAGETA | | ggacgcggcagccctcgagcacgacaa |
| | | PGKKRPLIESPQQPDSSTGI | | ggcctacgaccagcagctcaaggccggt |
| | | GKKGKQPAKKKLVFEDET | | gacaacccctacctcaagtacaaccacgc |
| | | GAGDGPPEGSTSGAMSDD | | cgacgcggagttccagcagcggcttcag |
| | | SEMASGGGAPMADNNEG | | ggcgacacatcgtttgggggcaacctcg |
| | | ADGVGNASGNVVHCDSTW | | gcagagcagtcttccaggccaaaaagag |
| | | LGDRVITTSTRTWALPTY | | ggttcttgaacctcttggtctggttgagcaa |
| | | NNHLYKQISSASTGASND | | gcgggtgagacggctcctggaaagaag |
| | | NHYFGYSTPWGYFDFNRF | | agaccgttgattgaatccccccagcagcc |
| | | HCHFSPRDWQRLINNNVV | | cgactcctccacgggtatcggcaaaaaa |
| | | GFRPKRLNFKLFNIQVKEV | | ggcaagcagccggctaaaaagaagctc |
| | | TTNDGVTTIANNLTSTVQ | | gttttcgaagacgaaactggagcaggcg |
| | | VFSDSEYQLPYVLGSAHQ | | acggaccccctgagggatcaacttccgg |
| | | GCLPPFPADVFMIPQYGY | | agccatgtctgatgacagtgagatggcttc |
| | | LTLNNGSQAVGRSSFYCL | | aggcggtggcgcaccaatggcagacaat |
| | | EYFPSQMLRTGNNFTFSY | | aacgaaggcgccgacggagtgggtaat |
| | | TFEDVPFHSSYAHSQSLDR | | gcctcaggaaattggcattgcgattccaca |
| | | LMNPLIDQYLYYLNRTQN | | tggctgggcgacagagtcatcaccacca |
| | | QSGSAQNKDLLFSRGSPA | | gcacccgaacatgggccttgcccacctat |
| | | GMSVQPKNWLPGPCYRQ | | aacaaccacctctacaagcaaatctccagt |
| | | QRVSKTKTDNNNSNFTWT | | gcttcaacgggggccagcaacgacaacc |
| | | GASKYNLNGRESIINPGTA | | actacttcggctacagcaccccctggggg |
| | | MASHKDDKDKFFPMSGV | | tattttgatttcaacagattccactgccatttc |
| | | MIFGKESAGASNTALDNV | | tcaccacgtgactggcagcgactcatcaa |
| | | MITDEEEIKATNPVATERF | | caacaattggggattccggcccaagaga |
| | | GTVAVNLQSSSTDPATGD | | ctcaacttcaagctcttcaacatccaagtca |
| | | VHVMGALPGMVWQDRD | | aggaggtcacgacgaatgatggcgtcac |
| | | VYLQGPIWAKIPHTDGHF | | gaccatcgctaataaccttaccagcacgg |
| | | HPSPLMGGFGLKHPPPQIL | | ttcaagtcttctcggactcggagtaccagtt |
| | | IKNTPVPANPPAEFSATKF | | gccgtacgtcctcggctctgcgcaccagg |
| | | ASFITQYSTGQVSVEIEWE | | gctgcctccctccgttcccggcggacgtg |
| | | LQKENSKRWNPEVQYTSN | | ttcatgattccgcagtacggctacctaacg |
| | | YAKSANVDFTVDNNGLY | | ctcaacaatggcagccaggcagtgggac |
| | | TEPRPIGTRYLTRPL | | ggtcatccttttactgcctggaatatttccca |
| | | | | tcgcagatgctgagaacgggcaataactt |
| | | | | taccttcagctacaccttcgaggacgtgc |
| | | | | ctttccacagcagctacgcgcacagccag |
| | | | | agcctggaccggctgatgaatcctctcatc |
| | | | | gaccagtacctgtattacctgaacagaact |
| | | | | cagaatcagtccggaagtgcccaaaaca |
| | | | | aggacttgctgtttagccgggggtctcca |
| | | | | gctggcatgtctgttcagcccaaaaactg |
| | | | | gctacctggaccctgttaccggcagcagc |
| | | | | gcgtttctaaaacaaaaacagacaacaac |
| | | | | aacagcaactttacctggactggtgcttca |
| | | | | aaatataaccttaatgggcgtgaatctataa |
| | | | | tcaaccctggcactgctatggcctcacac |
| | | | | aaagacgacaaagacaagttctttcccat |
| | | | | gagcggtgtcatgatttttggaaaggaga |
| | | | | gcgccggagcttcaaacactgcattggac |
| | | | | aatgtcatgatcacagacgaagaggaaat |
| | | | | caaagccactaaccccgtggccaccgaa |
| | | | | agatttgggactgtggcagtcaatctccag |
| | | | | agcagcagcacagaccctgcgaccgga |
| | | | | gatgtgcatgttatgggagccttacctgga |
| | | | | atggtgtggcaagacagagacgtatacct |
| | | | | gcagggtcctatagggccaaaattcctca |
| | | | | cacggatggacactttcacccgtctcctct |
| | | | | catgggcggctaggacttaagcacccgc |
| | | | | ctcctcagatcctcatcaaaaacacgcctg |
| | | | | ttcctgcgaatcctccggcagagtatcgg |
| | | | | ctacaaagtagcttcattcatcacccagtat |
| | | | | tccacaggacagtgagcgtggagattga |
| | | | | atgggagctgcagaaagaaaacagcaaa |
| | | | | cgctggaatcccgaagtgcagtatacatct |
| | | | | aactatgcaaaatctgccaacgttgatttca |
| | | | | ctgtggacaacaatggactttatactgagc |
| | | | | ctcgccccattggcacccgttacctcaccc |
| | | | | gtcccctgtaa |
| Chimera 4 | 46 | MSFVDHPPDWLEEVGEGL | 53 | atgtcttttgttgatcaccctccagattggtt |
| rAAV5VP1/2- | | REFLGLEAGPPKPKPNQQ | | ggaagaagttggtgaaggtcttcgcgag |
| AAV6VP3 | | HQDQARGLVLPGYNYLG | | tttagggccttgaagcgggcccaccgaa |
| | | PGNGLDRGEPVNRADEVA | | accaaaacccaatcagcagcatcaagatc |
| | | REHDISYNEQLEAGDNPY | | aagcccgtggtcttgtgctgcctggttata |
| | | LKYNHADAEFQEKLADD | | actatctcggacccggaaacggtctcgat |
| | | TSFGGNLGKAVFQAKKRV | | cgaggagagcctgtcaacagggcagac |
| | | LEPFGLVEEGAKTAPTGK | | gaggtcgcgcgagagcacgacatctcgt |
| | | RIDDHFPKRKKARTEEDS | | acaacgagcagcttgaggcgggagaca |
| | | KPSTSSDAEAGPSGSQQL | | acccctacctcaagtacaaccacgcggac |
| | | QIPAQPASSLGADTMASG | | gccgagtttcaggagaagctcgccgacg |
| | | GGAPMADNNEGADGVGN | | acacatccttcgggggaaacctcggaaa |
| | | ASGNWHCDSTWLGDRVI | | ggcagtctttcaggccaagaaaagggttc |
| | | TTSTRTWALPTYNNHLYK | | tcgaaccttaggcctggttgaagagggtg |
| | | QISSASTGASNDNHYFGYS | | ctaagacggcccctaccggaaagcggat |
| | | TPWGYFDFNRFHCHFSPR | | agacgaccactaccaaaaagaaagaag |
| | | DWQRLINNNVVGFRPKRL | | gctcggaccgaagaggactccaagcctt |
| | | NFKLFNIQVKEVTTNDGV | | ccacctcgtcagacgccgaagctggacc |
| | | TTIANNLTSTVQVFSDSEY | | cagcggatcccagcagctgcaaatccca |
| | | QLPYVLGSAHQGCLPPFP | | gcccaaccagcctcaagtagggagctga |
| | | ADVFMIPQYGYLTLNNGS | | tacaatggcttcaggcggtggcgcaccaa |
| | | QAVGRSSFYCLEYFPSQM | | tggcagacaataacgaaggcgccgacg |
| | | LRTGNNFTFSYTFEDVPFH | | gagtgggtaatgcctcaggaaattggcatt |
| | | SSYAHSQSLDRLMNPLID | | gcgattccacatggctgggcgacagagt |
| | | QYLYYLNRTQNQSGSAQ | | catcaccaccagcacccgaacatgggcc |
| | | NKDLLFSRGSPAGMSVQP | | ttgcccacctataacaaccacctctacaag |
| | | KNWLPGPCYRQQRVSKT | | caaatctccagtgcttcaacgggggccag |
| | | KTDNNNSNFTWTGASKY | | caacgacaaccactacttcggctacagca |
| | | NLNGRESIINPGTAMASHK | | ccccctgggggtattttgatacaacagatt |
| | | DDKDKFFPMSGVMIFGKE | | ccactgccatttctcaccacgtgactggca |
| | | SAGASNTALDNVMITDEE | | gcgactcatcaacaacaattggggattcc |
| | | EIKATNPVATERFGTVAV | | ggcccaagagactcaacttcaagctcttc |
| | | NLQSSSTDPATGDVHVMG | | aacatccaagtcaaggaggtcacgacga |
| | | ALPGMVWQDRDVYLQGP | | atgatggcgtcacgaccatcgctaataac |
| | | IWAKIPHTDGHFHPSPLM | | cttaccagcacggttcaagtcttctcggac |
| | | GGFGLKHPPPQILIKNTPV | | tcggagtaccagttgccgtacgtcctcgg |
| | | PANPPAEFSATKFASFITQ | | ctctgcgcaccagggctgcctccctccgtt |
| | | YSTGQVSVEIEWELQKEN | | cccggcggacgtgttcatgattccgcagt |
| | | SKRWNPEVQYTSNYAKS | | acggctacctaacgctcaacaatggcagc |
| | | ANVDFTVDNNGLYTEPRP | | caggcagtgggacggtcatccttttactgc |
| | | IGTRYLTRPL | | ctggaatatttcccatcgcagatgctgaga |
| | | | | acgggcaataactttaccttcagctacacc |
| | | | | ttcgaggacgtgcctttccacagcagctac |
| | | | | gcgcacagccagagcctggaccggctg |
| | | | | atgaatcctctcatcgaccagtacctgtatt |
| | | | | acctgaacagaactcagaatcagtccgga |
| | | | | agtgcccaaaacaaggacttgctgtttagc |
| | | | | cgggggtctccagctggcatgtctgttca |
| | | | | gcccaaaaactggctacctggaccctgtt |
| | | | | accggcagcagcgcgtttctaaaacaaaa |
| | | | | acagacaacaacaacagcaactttacctg |
| | | | | gactggtgcttcaaaatataaccttaatgg |
| | | | | gcgtgaatctataatcaaccctggcactgc |
| | | | | tatggcctcacacaaagacgacaaagac |
| | | | | aagttctttcccatgagcggtgtcatgatttt |
| | | | | tggaaaggagagcgccggagcttcaaac |
| | | | | actgcattggacaatgtcatgatcacagac |
| | | | | gaagaggaaatcaaagccactaaccccg |
| | | | | tggccaccgaaagatttgggactgtggca |
| | | | | gtcaatctccagagcagcagcacagacc |
| | | | | ctgcgaccggagatgtgcatgttatggga |
| | | | | gccttacctggaatggtgtggcaagacag |
| | | | | agacgtatacctgcagggtcctatttgggc |
| | | | | caaaattcctcacacggatggacactttca |
| | | | | cccgtctcctctcatgggcggctttggactt |
| | | | | aagcacccgcctcctcagatcctcatcaa |
| | | | | aaacacgcctgttcctgcgaatcctccgg |
| | | | | cagagttttcggctacaaagtttgcttcattc |
| | | | | atcacccagtattccacaggacaagtgag |
| | | | | cgtggagattgaatgggagctgcagaaa |
| | | | | gaaaacagcaaacgctggaatcccgaag |
| | | | | tgcagtatacatctaactatgcaaaatctgc |
| | | | | caacgttgatttcactgtggacaacaatgg |
| | | | | actttatactgagcctcgccccattggcac |
| | | | | ccgttacctcacccgtcccctgtaa |
| Chimera 5 | 47 | MAADGYLPDWLEDNLSE | 54 | atggctgctgacggttatcttccagattgg |
| rAAV11VP1/2- | | GIREWWDLKPGAPKPKA | | ctcgaggacaacctctctgagggcattcg |
| AAV6VP 3 | | NQQKQDDGRGLVLPGYK | | cgagtggtgggacctgaaacctggagcc |
| | | YLGPFNGLDKGEPVNAAD | | ccgaagcccaaggccaaccagcagaag |
| | | AAALEHDKAYDQQLKAG | | caggacgacggccggggtctggtgcttc |
| | | DNPYLRYNHADAEFQERL | | ctggctacaagtacctcggacccttcaac |
| | | QEDTSFGGNLGRAVFQAK | | ggactcgacaagggggagcccgtcaac |
| | | KRVLEPLGLVEEGAKTAP | | gcggcggacgcagcggccctcgagcac |
| | | GKKRPLESPQEPDSSSGIG | | gacaaggcctacgaccagcagctcaaag |
| | | KKGKQPARKRLNFEEDTG | | cgggtgacaatccgtacctgcggtataac |
| | | AGDGPPEGSDTSAMSSDIE | | cacgccgacgccgagtttcaggagcgtct |
| | | MASGGGAPMADNNEGAD | | gcaagaagatacgtcttttgggggcaacc |
| | | GVGNASGNVVHCDSTWLG | | tcgggcgagcagtcttccaggccaagaa |
| | | DRVITTSTRTWALPTYNN | | gagggtactcgaacctctgggcctggttg |
| | | HLYKQISSASTGASNDNH | | aagaaggtgctaaaacggctcctggaaa |
| | | YFGYSTPWGYFDFNRFHC | | gaagagaccgttagagtcaccacaagag |
| | | HFSPRDWQRLINNNWGFR | | cccgactcctcctcgggcatcggcaaaaa |
| | | PKRLNFKLFNIQVKEVTTN | | aggcaaacaaccagccagaaagaggct |
| | | DGVTTIANNLTSTVQVFS | | caactttgaagaggacactggagccgga |
| | | DSEYQLPYVLGSAHQGCL | | gacggaccccctgaaggatcagatacca |
| | | PPFPADVFMIPQYGYLTLN | | gcgccatgtcttcagacattgaaatggctt |
| | | NGSQAVGRSSFYCLEYFP | | caggcggtggcgcaccaatggcagaca |
| | | SQMLRTGNNFTFSYTFED | | ataacgaaggcgccgacggagtgggtaa |
| | | VPFHSSYAHSQSLDRLMN | | tgcctcaggaaattggcattgcgattccac |
| | | PLIDQYLYYLNRTQNQSG | | atggctgggcgacagagtcatcaccacc |
| | | SAQNKDLLFSRGSPAGMS | | agcacccgaacatgggccttgcccaccta |
| | | VQPKNVVLPGPCYRQQRV | | taacaaccacctctacaagcaaatctccag |
| | | SKTKTDNNNSNFTWTGAS | | tgcttcaacgggggccagcaacgacaac |
| | | KYNLNGRESIINPGTAMAS | | cactacttcggctacagcaccccctgggg |
| | | HKDDKDKFFPMSGVMIFG | | gtattttgatacaacagattccactgccattt |
| | | KESAGASNTALDNVMITD | | ctcaccacgtgactggcagcgactcatca |
| | | EEEIKATNPVATERFGTVA | | acaacaattggggattccggcccaagag |
| | | VNLQSSSTDPATGDVHVM | | actcaacttcaagctcttcaacatccaagtc |
| | | GALPGMVWQDRDVYLQG | | aaggaggtcacgacgaatgatggcgtca |
| | | PIWAKIPHTDGHFHPSPLM | | cgaccatcgctaataaccttaccagcacg |
| | | GGFGLKHPPPQILIKNTPV | | gttcaagtcttctcggactcggagtaccag |
| | | PANPPAEFSATKFASFITQ | | ttgccgtacgtcctcggctctgcgcacca |
| | | YSTGQVSVEIEWELQKEN | | gggctgcctccctccgttcccggcggac |
| | | SKRWNPEVQYTSNYAKS | | gtgttcatgattccgcagtacggctaccta |
| | | ANVDFTVDNNGLYTEPRP | | acgctcaacaatggcagccaggcagtgg |
| | | IGTRYLTRPL | | gacggtcatccttttactgcctggaatatac |
| | | | | ccatcgcagatgctgagaacgggcaata |
| | | | | actttaccttcagctacaccttcgaggacgt |
| | | | | gcctaccacagcagctacgcgcacagcc |
| | | | | agagcctggaccggctgatgaatcctctc |
| | | | | atcgaccagtacctgtattacctgaacaga |
| | | | | actcagaatcagtccggaagtgcccaaaa |
| | | | | caaggacttgctgatagccgggggtctcc |
| | | | | agctggcatgtctgttcagcccaaaaactg |
| | | | | gctacctggaccctgttaccggcagcagc |
| | | | | gcgtactaaaacaaaaacagacaacaac |
| | | | | aacagcaactttacctggactggtgcttca |
| | | | | aaatataaccttaatgggcgtgaatctataa |
| | | | | tcaaccctggcactgctatggcctcacac |
| | | | | aaagacgacaaagacaagttctacccat |
| | | | | gagcggtgtcatgatttttggaaaggaga |
| | | | | gcgccggagcttcaaacactgcattggac |
| | | | | aatgtcatgatcacagacgaagaggaaat |
| | | | | caaagccactaaccccgtggccaccgaa |
| | | | | agatttgggactgtggcagtcaatctccag |
| | | | | agcagcagcacagaccctgcgaccgga |
| | | | | gatgtgcatgttatgggagccttacctgga |
| | | | | atggtgtggcaagacagagacgtatacct |
| | | | | gcagggtcctatagggccaaaattcctca |
| | | | | cacggatggacactttcacccgtctcctct |
| | | | | catgggcggctaggacttaagcacccgc |
| | | | | ctcctcagatcctcatcaaaaacacgcctg |
| | | | | ttcctgcgaatcctccggcagagtatcgg |
| | | | | ctacaaagtagcttcattcatcacccagtat |
| | | | | tccacaggacaagtgagcgtggagattg |
| | | | | aatgggagctgcagaaagaaaacagcaa |
| | | | | acgctggaatcccgaagtgcagtatacat |
| | | | | ctaactatgcaaaatctgccaacgagata |
| | | | | cactgtggacaacaatggactttatactga |
| | | | | gcctcgccccattggcacccgttacctca |
| | | | | cccgtcccctgtaa |
| Chimera 6 | 48 | MAADGYLPDWLEDNLSE | 55 | atggctgctgacggttatcttccagattgg |
| AAV12VP1/2- | | GIREWWALKPGAPQPKA | | ctcgaggacaacctctctgaaggcattcg |
| AAV6VP3 | | NQQHQDNGRGLVLPGYK | | cgagtggtgggcgctgaaacctggagct |
| | | YLGPFNGLDKGEPVNEAD | | ccacaacccaaggccaaccaacagcatc |
| | | AAALEHDKAYDKQLEQG | | aggacaacggcaggggtcttgtgcttcct |
| | | DNPYLKYNHADAEFQQR | | gggtacaagtacctcggacccttcaacgg |
| | | LATDTSFGGNLGRAVFQA | | actcgacaagggagagccggtcaagag |
| | | KKRILEPLGLVEEGVKTAP | | gcagacgccgcggccctcgagcacgac |
| | | GKKRPLEKTPNRPTNPDS | | aaggcctacgacaagcagctcgagcag |
| | | GKAPAKKKQKDGEPADS | | ggggacaacccgtatctcaagtacaacca |
| | | ARRTLDFEDSGAGDGPPE | | cgccgacgccgagttccagcagcgcttg |
| | | GSSSGEMSHDAEMASGG | | gcgaccgacacctcttagggggcaacct |
| | | GAPMADNNEGADGVGNA | | cgggcgagcagtcttccaggccaaaaag |
| | | SGNWHCDSTWLGDRVITT | | aggattctcgagcctctgggtctggttgaa |
| | | STRTWALPTYNNHLYKQI | | gagggcgttaaaacggctcctggaaaga |
| | | SSASTGASNDNHYFGYST | | aacgcccattagaaaagactccaaatcgg |
| | | PWGYFDFNRFHCHFSPRD | | ccgaccaacccggactctgggaaggccc |
| | | WQRLINNNVVGFRPKRLNF | | cggccaagaaaaagcaaaaagacggcg |
| | | KLFNIQVKEVTTNDGVTTI | | aaccagccgactctgctagaaggacactc |
| | | ANNLTSTVQVFSDSEYQL | | gactttgaagactctggagcaggagacg |
| | | PYVLGSAHQGCLPPFPAD | | gaccccctgagggatcatcttccggagaa |
| | | VFMIPQYGYLTLNNGSQA | | atgtctcatgatgctgagatggcttcaggc |
| | | VGRSSFYCLEYFPSQMLR | | ggtggcgcaccaatggcagacaataacg |
| | | TGNNFTFSYTFEDVPFHSS | | aaggcgccgacggagtgggtaatgcctc |
| | | YAHSQSLDRLMNPLIDQY | | aggaaattggcattgcgattccacatggct |
| | | LYYLNRTQNQSGSAQNK | | gggcgacagagtcatcaccaccagcacc |
| | | DLLFSRGSPAGMSVQPKN | | cgaacatgggccttgcccacctataacaa |
| | | WLPGPCYRQQRVSKTKTD | | ccacctctacaagcaaatctccagtgcttc |
| | | NNNSNFTWTGASKYNLN | | aacgggggccagcaacgacaaccacta |
| | | GRESIINPGTAMASHKDD | | cttcggctacagcaccccctgggggtattt |
| | | KDKFFPMSGVMIFGKESA | | tgatttcaacagattccactgccatttctcac |
| | | GASNTALDNVMITDEEEI | | cacgtgactggcagcgactcatcaacaac |
| | | KATNPVATERFGTVAVNL | | aattggggattccggcccaagagactcaa |
| | | QSSSTDPATGDVHVMGAL | | cttcaagctcttcaacatccaagtcaagga |
| | | PGMVWQDRDVYLQGPIW | | ggtcacgacgaatgatggcgtcacgacc |
| | | AKIPHTDGHFHPSPLMGG | | atcgctaataaccttaccagcacggttcaa |
| | | FGLKHPPPQILIKNTPVPA | | gtcttctcggactcggagtaccagttgccg |
| | | NPPAEFSATKFASFITQYS | | tacgtcctcggctctgcgcaccagggctg |
| | | TGQVSVEIEWELQKENSK | | cctccctccgttcccggcggacgtgttcat |
| | | RWNPEVQYTSNYAKSAN | | gattccgcagtacggctacctaacgctca |
| | | VDFTVDNNGLYTEPRPIGT | | acaatggcagccaggcagtgggacggtc |
| | | RYLTRPL | | atccttttactgcctggaatatttcccatcgc |
| | | | | agatgctgagaacgggcaataactttacct |
| | | | | tcagctacaccttcgaggacgtgcctttcc |
| | | | | acagcagctacgcgcacagccagagcct |
| | | | | ggaccggctgatgaatcctctcatcgacc |
| | | | | agtacctgtattacctgaacagaactcaga |
| | | | | atcagtccggaagtgcccaaaacaagga |
| | | | | cttgctgtttagccgggggtctccagctgg |
| | | | | catgtctgttcagcccaaaaactggctacc |
| | | | | tggaccctgttaccggcagcagcgcgttt |
| | | | | ctaaaacaaaaacagacaacaacaacag |
| | | | | caactttacctggactggtgcttcaaaatat |
| | | | | aaccttaatgggcgtgaatctataatcaac |
| | | | | cctggcactgctatggcctcacacaaaga |
| | | | | cgacaaagacaagttctttcccatgagcg |
| | | | | gtgtcatgatttttggaaaggagagcgcc |
| | | | | ggagcttcaaacactgcattggacaatgtc |
| | | | | atgatcacagacgaagaggaaatcaaag |
| | | | | ccactaaccccgtggccaccgaaagattt |
| | | | | gggactgtggcagtcaatctccagagca |
| | | | | gcagcacagaccctgcgaccggagatgt |
| | | | | gcatgttatgggagccttacctggaatggt |
| | | | | gtggcaagacagagacgtatacctgcag |
| | | | | ggtcctatttgggccaaaattcctcacacg |
| | | | | gatggacactttcacccgtctcctctcatg |
| | | | | ggcggctttggacttaagcacccgcctcc |
| | | | | tcagatcctcatcaaaaacacgcctgttcct |
| | | | | gcgaatcctccggcagagttttcggctac |
| | | | | aaagtttgcttcattcatcacccagtattcca |
| | | | | caggacaagtgagcgtggagattgaatg |
| | | | | ggagctgcagaaagaaaacagcaaacg |
| | | | | ctggaatcccgaagtgcagtatacatctaa |
| | | | | ctatgcaaaatctgccaacgttgatttcact |
| | | | | gtggacaacaatggactttatactgagcct |
| | | | | cgccccattggcacccgttacctcacccg |
| | | | | tcccctgtaa |
| Chimera 7 | 49 | MTDGYLPDWLEDNLSEG | 56 | atgactgacggttaccttccagattggcta |
| AAV4VP1u- | | VREWWALQPGAPKPKAN | | gaggacaacctctctgaaggcgttcgaga |
| AAV6VP2/3 | | QQHQDNARGLVLPGYKY | | gtggtgggcgctgcaacctggagcccct |
| | | LGPGNGLDKGEPVNAAD | | aaacccaaggcaaatcaacaacatcagg |
| | | AAALEHDKAYDQQLKAG | | acaacgctcggggtcttgtgcttccgggtt |
| | | DNPYLKYNHADAEFQQR | | acaaatacctcggacccggcaacggact |
| | | LQGDTSFGGNLGRAVFQA | | cgacaagggggaacccgtcaacgcagc |
| | | KKRVLEPLGLVEQAGETA | | ggacgcggcagccctcgagcacgacaa |
| | | PGKKRPVEQSPQEPDSSSG | | ggcctacgaccagcagctcaaggccggt |
| | | IGKTGQQPAKKRLNFGQT | | gacaacccctacctcaagtacaaccacgc |
| | | GDSESVPDPQPLGEPPATP | | cgacgcggagttccagcagcggcttcag |
| | | AAVGPTTMASGGGAPMA | | ggcgacacatcgtttgggggcaacctcg |
| | | DNNEGADGVGNASGNWH | | gcagagcagtcttccaggccaaaaagag |
| | | CDSTWLGDRVITTSTRTW | | ggttcttgaacctcttggtctggttgagcaa |
| | | ALPTYNNHLYKQISSASTG | | gcgggtgagacggctcctggaaagaaac |
| | | ASNDNHYFGYSTPWGYF | | gtccggtagagcagtcgccacaagagcc |
| | | DFNRFHCHFSPRDWQRLI | | agactcctcctcgggcattggcaagacag |
| | | NNNWGFRPKRLNFKLFNI | | gccagcagcccgctaaaaagagactcaa |
| | | QVKEVTTNDGVTTIANNL | | ttttggtcagactggcgactcagagtcagt |
| | | TSTVQVFSDSEYQLPYVL | | ccccgacccacaacctctcggagaacctc |
| | | GSAHQGCLPPFPADVFMIP | | cagcaacccccgctgctgtgggacctact |
| | | QYGYLTLNNGSQAVGRSS | | acaatggcttcaggcggtggcgcaccaat |
| | | FYCLEYFPSQMLRTGNNF | | ggcagacaataacgaaggcgccgacgg |
| | | TFSYTFEDVPFHSSYAHSQ | | agtgggtaatgcctcaggaaattggcattg |
| | | SLDRLMNPLIDQYLYYLN | | cgattccacatggctgggcgacagagtca |
| | | RTQNQSGSAQNKDLLFSR | | tcaccaccagcacccgaacatgggccttg |
| | | GSPAGMSVQPKNWLPGP | | cccacctataacaaccacctctacaagca |
| | | CYRQQRVSKTKTDNNNS | | aatctccagtgcttcaacgggggccagca |
| | | NFTWTGASKYNLNGRESII | | acgacaaccactacttcggctacagcacc |
| | | NPGTAMASHKDDKDKFFP | | ccctgggggtattttgatttcaacagattcc |
| | | MSGVMIFGKESAGASNTA | | actgccatttctcaccacgtgactggcagc |
| | | LDNVMITDEEEIKATNPV | | gactcatcaacaacaattggggattccgg |
| | | ATERFGTVAVNLQSSSTD | | cccaagagactcaacttcaagctcttcaac |
| | | PATGDVHVMGALPGMV | | atccaagtcaaggaggtcacgacgaatg |
| | | WQDRDVYLQGPIWAKIPH | | atggcgtcacgaccatcgctaataacctta |
| | | TDGHFHPSPLMGGFGLKH | | ccagcacggttcaagtcttctcggactcg |
| | | PPPQILIKNTPVPANPPAEF | | gagtaccagttgccgtacgtcctcggctct |
| | | SATKFASFITQYSTGQVSV | | gcgcaccagggctgcctccctccgttccc |
| | | EIEWELQKENSKRWNPEV | | ggcggacgtgttcatgattccgcagtacg |
| | | QYTSNYAKSANVDFTVD | | gctacctaacgctcaacaatggcagccag |
| | | NNGLYTEPRPIGTRYLTRP | | gcagtgggacggtcatccttttactgcctg |
| | | L | | gaatatttcccatcgcagatgctgagaacg |
| | | | | ggcaataactttaccttcagctacaccttcg |
| | | | | aggacgtgcctttccacagcagctacgcg |
| | | | | cacagccagagcctggaccggctgatga |
| | | | | atcctctcatcgaccagtacctgtattacct |
| | | | | gaacagaactcagaatcagtccggaagt |
| | | | | gcccaaaacaaggacttgctgtttagccg |
| | | | | ggggtctccagctggcatgtctgttcagcc |
| | | | | caaaaactggctacctggaccctgttacc |
| | | | | ggcagcagcgcgtttctaaaacaaaaaca |
| | | | | gacaacaacaacagcaactttacctggac |
| | | | | tggtgcttcaaaatataaccttaatgggcgt |
| | | | | gaatctataatcaaccctggcactgctatg |
| | | | | gcctcacacaaagacgacaaagacaagt |
| | | | | tctttcccatgagcggtgtcatgatttttgga |
| | | | | aaggagagcgccggagcttcaaacactg |
| | | | | cattggacaatgtcatgatcacagacgaa |
| | | | | gaggaaatcaaagccactaaccccgtgg |
| | | | | ccaccgaaagatttgggactgtggcagtc |
| | | | | aatctccagagcagcagcacagaccctg |
| | | | | cgaccggagatgtgcatgttatgggagcc |
| | | | | ttacctggaatggtgtggcaagacagaga |
| | | | | cgtatacctgcagggtcctatttgggccaa |
| | | | | aattcctcacacggatggacactttcaccc |
| | | | | gtctcctctcatgggcggctttggacttaa |
| | | | | gcacccgcctcctcagatcctcatcaaaa |
| | | | | acacgcctgttcctgcgaatcctccggca |
| | | | | gagttttcggctacaaagtttgcttcattcat |
| | | | | cacccagtattccacaggacaagtgagc |
| | | | | gtggagattgaatgggagctgcagaaag |
| | | | | aaaacagcaaacgctggaatcccgaagt |
| | | | | gcagtatacatctaactatgcaaaatctgc |
| | | | | caacgttgatttcactgtggacaacaatgg |
| | | | | actttatactgagcctcgccccattggcac |
| | | | | ccgttacctcacccgtc |
| Chimera 8 | 50 | MAADGYLPDWLEDNLSE | 57 | atggctgctgacggttatcttccagattgg |
| AAV12VP1u- | | GIREWWALKPGAPQPKA | | ctcgaggacaacctctctgaaggcattcg |
| AAV6VP2/3 | | NQQHQDNGRGLVLPGYK | | cgagtggtgggcgctgaaacctggagct |
| | | YLGPFNGLDKGEPVNEAD | | ccacaacccaaggccaaccaacagcatc |
| | | AAALEHDKAYDKQLEQG | | aggacaacggcaggggtcttgtgcttcct |
| | | DNPYLKYNHADAEFQQR | | gggtacaagtacctcggacccttcaacgg |
| | | LATDTSFGGNLGRAVFQA | | actcgacaagggagagccggtcaacga |
| | | KKRILEPLGLVEEGVKTAP | | ggcagacgccgcggccctcgagcacga |
| | | GKKRPVEQSPQEPDSSSGI | | caaggcctacgacaagcagctcgagcag |
| | | GKTGQQPAKKRLNFGQT | | ggggacaacccgtatctcaagtacaacca |
| | | GDSESVPDPQPLGEPPATP | | cgccgacgccgagttccagcagcgcttg |
| | | AAVGPTTMASGGGAPMA | | gcgaccgacacctcttttgggggcaacct |
| | | DNNEGADGVGNASGNWH | | cgggcgagcagtcttccaggccaaaaag |
| | | CDSTWLGDRVITTSTRTW | | aggattctcgagcctctgggtctggttgaa |
| | | ALPTYNNHLYKQISSASTG | | gagggcgttaaaacggctcctggaaaga |
| | | ASNDNHYFGYSTPWGYF | | aacgtccggtagagcagtcgccacaaga |
| | | DFNRFHCHFSPRDWQRLI | | gccagactcctcctcgggcattggcaaga |
| | | NNNWGFRPKRLNFKLFNI | | caggccagcagcccgctaaaaagagact |
| | | QVKEVTTNDGVTTIANNL | | caattttggtcagactggcgactcagagtc |
| | | TSTVQVFSDSEYQLPYVL | | agtccccgacccacaacctctcggagaa |
| | | GSAHQGCLPPFPADVFMIP | | cctccagcaacccccgctgctgtgggac |
| | | QYGYLTLNNGSQAVGRSS | | ctactacaatggcttcaggcggtggcgca |
| | | FYCLEYFPSQMLRTGNNF | | ccaatggcagacaataacgaaggcgccg |
| | | TFSYTFEDVPFHSSYAHSQ | | acggagtgggtaatgcctcaggaaattgg |
| | | SLDRLMNPLIDQYLYYLN | | cattgcgattccacatggctgggcgacag |
| | | RTQNQSGSAQNKDLLFSR | | agtcatcaccaccagcacccgaacatgg |
| | | GSPAGMSVQPKNWLPGP | | gccttgcccacctataacaaccacctctac |
| | | CYRQQRVSKTKTDNNNS | | aagcaaatctccagtgcttcaacgggggc |
| | | NFTWTGASKYNLNGRESII | | cagcaacgacaaccactacttcggctaca |
| | | NPGTAMASHK | | gcaccccctgggggtattttgatttcaaca |
| | | DDKDKFFPMSGVMIFGKE | | gattccactgccatttctcaccacgtgactg |
| | | SAGASNTALDNVMITDEE | | gcagcgactcatcaacaacaattggggat |
| | | EIKATNPVATERFGTVAV | | tccggcccaagagactcaacttcaagctc |
| | | NLQSSSTDPATGDVHVMG | | ttcaacatccaagtcaaggaggtcacgac |
| | | ALPGMVWQDRDVYLQGP | | gaatgatggcgtcacgaccatcgctaata |
| | | IWAKIPHTDGHFHPSPLM | | accttaccagcacggttcaagtcttctcgg |
| | | GGFGLKHPPPQILIKNTPV | | actcggagtaccagttgccgtacgtcctc |
| | | PANPPAEFSATKFASFITQ | | ggctctgcgcaccagggctgcctccctcc |
| | | YSTGQVSVEIEWELQKEN | | gttcccggcggacgtgttcatgattccgca |
| | | SKRWNPEVQYTSNYAKS | | gtacggctacctaacgctcaacaatggca |
| | | ANVDFTVDNNGLYTEPRP | | gccaggcagtgggacggtcatccttttact |
| | | IGTRYLTRPL | | gcctggaatatttcccatcgcagatgctga |
| | | | | gaacgggcaataactttaccttcagctaca |
| | | | | ccttcgaggacgtgcctaccacagcagct |
| | | | | acgcgcacagccagagcctggaccggc |
| | | | | tgatgaatcctctcatcgaccagtacctgta |
| | | | | ttacctgaacagaactcagaatcagtccg |
| | | | | gaagtgcccaaaacaaggacttgctgttta |
| | | | | gccgggggtctccagctggcatgtctgtt |
| | | | | cagcccaaaaactggctacctggaccctg |
| | | | | ttaccggcagcagcgcgtttctaaaacaa |
| | | | | aaacagacaacaacaacagcaactttacc |
| | | | | tggactggtgcttcaaaatataaccttaatg |
| | | | | ggcgtgaatctataatcaaccctggcactg |
| | | | | ctatggcctcacacaaagacgacaaaga |
| | | | | caagttctacccatgagcggtgtcatgatt |
| | | | | tttggaaaggagagcgccggagcttcaa |
| | | | | acactgcattggacaatgtcatgatcacag |
| | | | | acgaagaggaaatcaaagccactaaccc |
| | | | | cgtggccaccgaaagatagggactgtgg |
| | | | | cagtcaatctccagagcagcagcacaga |
| | | | | ccctgcgaccggagatgtgcatgttatgg |
| | | | | gagccttacctggaatggtgtggcaagac |
| | | | | agagacgtatacctgcagggtcctatagg |
| | | | | gccaaaattcctcacacggatggacacttt |
| | | | | cacccgtctcctctcatgggcggctagga |
| | | | | cttaagcacccgcctcctcagatcctcatc |
| | | | | aaaaacacgcctgttcctgcgaatcctcc |
| | | | | ggcagagttttcggctacaaagtagcttca |
| | | | | ttcatcacccagtattccacaggacaagtg |
| | | | | agcgtggagattgaatgggagctgcaga |
| | | | | aagaaaacagcaaacgctggaatcccga |
| | | | | agtgcagtatacatctaactatgcaaaatct |
| | | | | gccaacgttgatttcactgtggacaacaat |
| | | | | ggactttatactgagcctcgccccattggc |
| | | | | acccgttacctcacccgtcccctgtaa |
| Chimera 7b | | | 58 | ggtaccaaaacaaatgttctcgtcacgtgg |
| AAV4VP1u- | | | | gcatgaatctgatgctgtttccctgcagac |
| AAV6VP2/3 | | | | aatgcgagagaatgaatcagaattcaaat |
| | | | | atctgcttcactcacggacagaaagactgt |
| | | | | ttagagtgctttcccgtgtcagaatctcaac |
| | | | | ccgtttctgtcgtcaaaaaggcgtatcaga |
| | | | | aactgtgctacattcatcatatcatgggaaa |
| | | | | ggtgccagacgcttgcactgcctgcgatc |
| | | | | tggtcaatgtggatttggatgactgcatcttt |
| | | | | gaacaataaatgatttaaatcaggtatgact |
| | | | | gacggttaccttccagattggctagagga |
| | | | | caacctctctgaaggcgttcgagagtggt |
| | | | | gggcgctgcaacctggagcccctaaacc |
| | | | | caaggcaaatcaacaacatcaggacaac |
| | | | | gctcggggtcttgtgcttccgggttacaaa |
| | | | | tacctcggacccggcaacggactcgaca |
| | | | | agggggaacccgtcaacgcagcggacg |
| | | | | cggcagccctcgagcacgacaaggccta |
| | | | | cgaccagcagctcaaggccggtgacaac |
| | | | | ccctacctcaagtacaaccacgccgacgc |
| | | | | ggagttccagcagcggcttcagggcgac |
| | | | | acatcgtttgggggcaacctcggcagag |
| | | | | cagtcttccaggccaaaaagagggttctt |
| | | | | gaacctcttggtctggttgagcaagcggg |
| | | | | tgagacggctcctggaaagaaacgtccg |
| | | | | gtagagcagtcgccacaagagccagact |
| | | | | cctcctcgggcattggcaagacaggcca |
| | | | | gcagcccgctaaaaagagactcaattttg |
| | | | | gtcagactggcgactcagagtcagtcccc |
| | | | | gacccacaacctctcggagaacctccag |
| | | | | caacccccgctgctgtgggacctactaca |
| | | | | atggcttcaggcggtggcgcaccaatgg |
| | | | | cagacaataacgaaggcgccgacggag |
| | | | | tgggtaatgcctcaggaaattggcattgcg |
| | | | | attccacatggctgggcgacagagtcatc |
| | | | | accaccagcacccgaacatgggccttgc |
| | | | | ccacctataacaaccacctctacaagcaa |
| | | | | atctccagtgcttcaacgggggccagcaa |
| | | | | cgacaaccactacttcggctacagcaccc |
| | | | | cctgggggtattttgatttcaacagattcca |
| | | | | ctgccatttctcaccacgtgactggcagcg |
| | | | | actcatcaacaacaattggggattccggc |
| | | | | ccaagagactcaacttcaagctcttcaaca |
| | | | | tccaagtcaaggaggtcacgacgaatgat |
| | | | | ggcgtcacgaccatcgctaataaccttac |
| | | | | cagcacggttcaagtcttctcggactcgg |
| | | | | agtaccagttgccgtacgtcctcggctctg |
| | | | | cgcaccagggctgcctccctccgttcccg |
| | | | | gcggacgtgttcatgattccgcagtacgg |
| | | | | ctacctaacgctcaacaatggcagccagg |
| | | | | cagtgggacggtcatccttttactgcctgg |
| | | | | aatatttcccatcgcagatgctgagaacgg |
| | | | | gcaataactttaccttcagctacaccttcga |
| | | | | ggacgtgcctttccacagcagctacgcgc |
| | | | | acagccagagcctggaccggctgatgaa |
| | | | | tcctctcatcgaccagtacctgtattacctg |
| | | | | aacagaactcagaatcagtccggaagtg |
| | | | | cccaaaacaaggacttgctgtttagccgg |
| | | | | gggtctccagctggcatgtctgttcagccc |
| | | | | aaaaactggctacctggaccctgttaccg |
| | | | | gcagcagcgcgtttctaaaacaaaaacag |
| | | | | acaacaacaacagcaactttacctggact |
| | | | | ggtgcttcaaaatataaccttaatgggcgt |
| | | | | gaatctataatcaaccctggcactgctatg |
| | | | | gcctcacacaaagacgacaaagacaagt |
| | | | | tctttcccatgagcggtgtcatgatttttgga |
| | | | | aaggagagcgccggagcttcaaacactg |
| | | | | cattggacaatgtcatgatcacagacgaa |
| | | | | gaggaaatcaaagccactaaccccgtgg |
| | | | | ccaccgaaagatttgggactgtggcagtc |
| | | | | aatctccagagcagcagcacagaccctg |
| | | | | cgaccggagatgtgcatgttatgggagcc |
| | | | | ttacctggaatggtgtggcaagacagaga |
| | | | | cgtatacctgcagggtcctatttgggccaa |
| | | | | aattcctcacacggatggacactttcaccc |
| | | | | gtctcctctcatgggcggctttggacttaa |
| | | | | gcacccgcctcctcagatcctcatcaaaa |
| | | | | acacgcctgttcctgcgaatcctccggca |
| | | | | gagttttcggctacaaagtttgcttcattcat |
| | | | | cacccagtattccacaggacaagtgagc |
| | | | | gtggagattgaatgggagctgcagaaag |
| | | | | aaaacagcaaacgctggaatcccgaagt |
| | | | | gcagtatacatctaactatgcaaaatctgc |
| | | | | caacgttgatttcactgtggacaacaatgg |
| | | | | actttatactgagcctcgccccattggcac |
| | | | | ccgttacctcacccgtcccctgtaattgtgt |
| | | | | gttaatcaataaaccggt |
| Chimera 2b | | | 59 | ggtaccaaaacaaatgttctcgtcacgtgg |
| AAV5VP1u- | | | | gcatgaatctgatgctgtttccctgcagac |
| AAV6VP2/3 | | | | aatgcgagagaatgaatcagaattcaaat |
| | | | | atctgcttcactcacggacagaaagactgt |
| | | | | ttagagtgctttcccgtgtcagaatctcaac |
| | | | | ccgtttctgtcgtcaaaaaggcgtatcaga |
| | | | | aactgtgctacattcatcatatcatgggaaa |
| | | | | ggtgccagacgcttgcactgcctgcgatc |
| | | | | tggtcaatgtggatttggatgactgcatcttt |
| | | | | gaacaataaatgatttaaatcaggtatgtct |
| | | | | tttgttgatcaccctccagattggttggaag |
| | | | | aagttggtgaaggtcttcgcgagtttttgg |
| | | | | gccttgaagcgggcccaccgaaaccaaa |
| | | | | acccaatcagcagcatcaagatcaagccc |
| | | | | gtggtcttgtgctgcctggttataactatctc |
| | | | | ggacccggaaacggtctcgatcgaggag |
| | | | | agcctgtcaacagggcagacgaggtcgc |
| | | | | gcgagagcacgacatctcgtacaacgag |
| | | | | cagcttgaggcgggagacaacccctacc |
| | | | | tcaagtacaaccacgcggacgccgagttt |
| | | | | caggagaagctcgccgacgacacatcct |
| | | | | tcgggggaaacctcggaaaggcagtcttt |
| | | | | caggccaagaaaagggttctcgaacctttt |
| | | | | ggcctggttgaagagggtgctaagacgg |
| | | | | ctcctggaaagaaacgtccggtagagca |
| | | | | gtcgccacaagagccagactcctcctcg |
| | | | | ggcattggcaagacaggccagcagccc |
| | | | | gctaaaaagagactcaattttggtcagact |
| | | | | ggcgactcagagtcagtccccgacccac |
| | | | | aacctctcggagaacctccagcaaccccc |
| | | | | gctgctgtgggacctactacaatggcttca |
| | | | | ggcggtggcgcaccaatggcagacaata |
| | | | | acgaaggcgccgacggagtgggtaatg |
| | | | | cctcaggaaattggcattgcgattccacat |
| | | | | ggctgggcgacagagtcatcaccaccag |
| | | | | cacccgaacatgggccttgcccacctata |
| | | | | acaaccacctctacaagcaaatctccagt |
| | | | | gcttcaacgggggccagcaacgacaacc |
| | | | | actacttcggctacagcaccccctggggg |
| | | | | tattttgatttcaacagattccactgccatttc |
| | | | | tcaccacgtgactggcagcgactcatcaa |
| | | | | caacaattggggattccggcccaagaga |
| | | | | ctcaacttcaagctcttcaacatccaagtca |
| | | | | aggaggtcacgacgaatgatggcgtcac |
| | | | | gaccatcgctaataaccttaccagcacgg |
| | | | | ttcaagtcttctcggactcggagtaccagtt |
| | | | | gccgtacgtcctcggctctgcgcaccagg |
| | | | | gctgcctccctccgttcccggcggacgtg |
| | | | | ttcatgattccgcagtacggctacctaacg |
| | | | | ctcaacaatggcagccaggcagtgggac |
| | | | | ggtcatccttttactgcctggaatatttccca |
| | | | | tcgcagatgctgagaacgggcaataactt |
| | | | | taccttcagctacaccttcgaggacgtgcc |
| | | | | tttccacagcagctacgcgcacagccaga |
| | | | | gcctggaccggctgatgaatcctctcatc |
| | | | | gaccagtacctgtattacctgaacagaact |
| | | | | cagaatcagtccggaagtgcccaaaaca |
| | | | | aggacttgctgtttagccgggggtctcca |
| | | | | gctggcatgtctgttcagcccaaaaactg |
| | | | | gctacctggaccctgttaccggcagcagc |
| | | | | gcgtttctaaaacaaaaacagacaacaac |
| | | | | aacagcaactttacctggactggtgcttca |
| | | | | aaatataaccttaatgggcgtgaatctataa |
| | | | | tcaaccctggcactgctatggcctcacac |
| | | | | aaagacgacaaagacaagactacccat |
| | | | | gagcggtgtcatgatttttggaaaggaga |
| | | | | gcgccggagcttcaaacactgcattggac |
| | | | | aatgtcatgatcacagacgaagaggaaat |
| | | | | caaagccactaaccccgtggccaccgaa |
| | | | | agatttgggactgtggcagtcaatctccag |
| | | | | agcagcagcacagaccctgcgaccgga |
| | | | | gatgtgcatgttatgggagccttacctgga |
| | | | | atggtgtggcaagacagagacgtatacct |
| | | | | gcagggtcctatagggccaaaattcctca |
| | | | | cacggatggacactttcacccgtctcctct |
| | | | | catgggcggctttggacttaagcacccgc |
| | | | | ctcctcagatcctcatcaaaaacacgcctg |
| | | | | ttcctgcgaatcctccggcagagtatcgg |
| | | | | ctacaaagtagcttcattcatcacccagtat |
| | | | | tccacaggacaagtgagcgtggagattg |
| | | | | aatgggagctgcagaaagaaaacagcaa |
| | | | | acgctggaatcccgaagtgcagtatacat |
| | | | | ctaactatgcaaaatctgccaacgagata |
| | | | | cactgtggacaacaatggactttatactga |
| | | | | gcctcgccccattggcacccgttacctca |
| | | | | cccgtcccctgtaattgtgtgaaatcaata |
| | | | | aaccggt |
| AAV11VP1u- | | | 60 | ggtaccaaaacaaatgactcgtcacgtgg |
| AAV6VP2/3 | | | | gcatgaatctgatgctgtaccctgcagac |
| | | | | aatgcgagagaatgaatcagaattcaaat |
| | | | | atctgcttcactcacggacagaaagactgt |
| | | | | ttagagtgctacccgtgtcagaatctcaac |
| | | | | ccgtactgtcgtcaaaaaggcgtatcaga |
| | | | | aactgtgctacattcatcatatcatgggaaa |
| | | | | ggtgccagacgcttgcactgcctgcgatc |
| | | | | tggtcaatgtggataggatgactgcatcta |
| | | | | gaacaataaatgatttaaatcaggtatggc |
| | | | | tgctgacggttatcttccagattggctcga |
| | | | | ggacaacctctctgagggcattcgcgagt |
| | | | | ggtgggacctgaaacctggagccccgaa |
| | | | | gcccaaggccaaccagcagaagcagga |
| | | | | cgacggccggggtctggtgcttcctggct |
| | | | | acaagtacctcggacccttcaacggactc |
| | | | | gacaagggggagcccgtcaacgcggcg |
| | | | | gacgcagcggccctcgagcacgacaag |
| | | | | gcctacgaccagcagctcaaagcgggtg |
| | | | | acaatccgtacctgcggtataaccacgcc |
| | | | | gacgccgagtttcaggagcgtctgcaag |
| | | | | aagatacgtcttagggggcaacctcggg |
| | | | | cgagcagtcttccaggccaagaagaggg |
| | | | | tactcgaacctctgggcctggttgaagaa |
| | | | | ggtgctaaaacggctcctggaaagaaac |
| | | | | gtccggtagagcagtcgccacaagagcc |
| | | | | agactcctcctcgggcattggcaagacag |
| | | | | gccagcagcccgctaaaaagagactcaa |
| | | | | ttttggtcagactggcgactcagagtcagt |
| | | | | ccccgacccacaacctctcggagaacctc |
| | | | | cagcaacccccgctgctgtgggacctact |
| | | | | acaatggcttcaggcggtggcgcaccaat |
| | | | | ggcagacaataacgaaggcgccgacgg |
| | | | | agtgggtaatgcctcaggaaattggcattg |
| | | | | cgattccacatggctgggcgacagagtca |
| | | | | tcaccaccagcacccgaacatgggccttg |
| | | | | cccacctataacaaccacctctacaagca |
| | | | | aatctccagtgcttcaacgggggccagca |
| | | | | acgacaaccactacttcggctacagcacc |
| | | | | ccctgggggtattttgatttcaacagattcc |
| | | | | actgccatttctcaccacgtgactggcagc |
| | | | | gactcatcaacaacaattggggattccgg |
| | | | | cccaagagactcaacttcaagctcttcaac |
| | | | | atccaagtcaaggaggtcacgacgaatg |
| | | | | atggcgtcacgaccatcgctaataacctta |
| | | | | ccagcacggttcaagtcttctcggactcg |
| | | | | gagtaccagttgccgtacgtcctcggctct |
| | | | | gcgcaccagggctgcctccctccgttccc |
| | | | | ggcggacgtgttcatgattccgcagtacg |
| | | | | gctacctaacgctcaacaatggcagccag |
| | | | | gcagtgggacggtcatccttttactgcctg |
| | | | | gaatatttcccatcgcagatgctgagaacg |
| | | | | ggcaataactttaccttcagctacaccttcg |
| | | | | aggacgtgcctttccacagcagctacgcg |
| | | | | cacagccagagcctggaccggctgatga |
| | | | | atcctctcatcgaccagtacctgtattacct |
| | | | | gaacagaactcagaatcagtccggaagt |
| | | | | gcccaaaacaaggacttgctgtttagccg |
| | | | | ggggtctccagctggcatgtctgttcagcc |
| | | | | caaaaactggctacctggaccctgttacc |
| | | | | ggcagcagcgcgtttctaaaacaaaaaca |
| | | | | gacaacaacaacagcaactttacctggac |
| | | | | tggtgcttcaaaatataaccttaatgggcgt |
| | | | | gaatctataatcaaccctggcactgctatg |
| | | | | gcctcacacaaagacgacaaagacaagt |
| | | | | tctttcccatgagcggtgtcatgatttttgga |
| | | | | aaggagagcgccggagcttcaaacactg |
| | | | | cattggacaatgtcatgatcacagacgaa |
| | | | | gaggaaatcaaagccactaaccccgtgg |
| | | | | ccaccgaaagatttgggactgtggcagtc |
| | | | | aatctccagagcagcagcacagaccctg |
| | | | | cgaccggagatgtgcatgttatgggagcc |
| | | | | ttacctggaatggtgtggcaagacagaga |
| | | | | cgtatacctgcagggtcctatttgggccaa |
| | | | | aattcctcacacggatggacactttcaccc |
| | | | | gtctcctctcatgggcggctttggacttaa |
| | | | | gcacccgcctcctcagatcctcatcaaaa |
| | | | | acacgcctgttcctgcgaatcctccggca |
| | | | | gagttttcggctacaaagtttgcttcattcat |
| | | | | cacccagtattccacaggacaagtgagc |
| | | | | gtggagattgaatgggagctgcagaaag |
| | | | | aaaacagcaaacgctggaatcccgaagt |
| | | | | gcagtatacatctaactatgcaaaatctgc |
| | | | | caacgttgatttcactgtggacaacaatgg |
| | | | | actttatactgagcctcgccccattggcac |
| | | | | ccgttacctcacccgtcccctgtaattgtgt |
| | | | | gttaatcaataaaccggt |
| Chimera 8b | | | 61 | ggtaccaaaacaaatgttctcgtcacgtgg |
| AAV12VP1u- | | | | gcatgaatctgatgctgtttccctgcagac |
| AAV6VP2/3 | | | | aatgcgagagaatgaatcagaattcaaat |
| | | | | atctgcttcactcacggacagaaagactgt |
| | | | | ttagagtgctttcccgtgtcagaatctcaac |
| | | | | ccgtttctgtcgtcaaaaaggcgtatcaga |
| | | | | aactgtgctacattcatcatatcatgggaaa |
| | | | | ggtgccagacgcttgcactgcctgcgatc |
| | | | | tggtcaatgtggatttggatgactgcatcttt |
| | | | | gaacaataaatgatttaaatcaggtatggc |
| | | | | tgctgacggttatcttccagattggctcga |
| | | | | ggacaacctctctgaaggcattcgcgagt |
| | | | | ggtgggcgctgaaacctggagctccaca |
| | | | | acccaaggccaaccaacagcatcaggac |
| | | | | aacggcaggggtcttgtgcttcctgggta |
| | | | | caagtacctcggacccttcaacggactcg |
| | | | | acaagggagagccggtcaacgaggcag |
| | | | | acgccgcggccctcgagcacgacaagg |
| | | | | cctacgacaagcagctcgagcaggggg |
| | | | | acaacccgtatctcaagtacaaccacgcc |
| | | | | gacgccgagttccagcagcgcttggcga |
| | | | | ccgacacctcttttgggggcaacctcggg |
| | | | | cgagcagtcttccaggccaaaaagagga |
| | | | | ttctcgagcctctgggtctggttgaagagg |
| | | | | gcgttaaaacggctcctggaaagaaacgt |
| | | | | ccggtagagcagtcgccacaagagcca |
| | | | | gactcctcctcgggcattggcaagacagg |
| | | | | ccagcagcccgctaaaaagagactcaatt |
| | | | | ttggtcagactggcgactcagagtcagtc |
| | | | | cccgacccacaacctctcggagaacctcc |
| | | | | agcaacccccgctgctgtgggacctacta |
| | | | | caatggcttcaggcggtggcgcaccaat |
| | | | | ggcagacaataacgaaggcgccgacgg |
| | | | | agtgggtaatgcctcaggaaattggcattg |
| | | | | cgattccacatggctgggcgacagagtca |
| | | | | tcaccaccagcacccgaacatgggccttg |
| | | | | cccacctataacaaccacctctacaagca |
| | | | | aatctccagtgcttcaacgggggccagca |
| | | | | acgacaaccactacttcggctacagcacc |
| | | | | ccctgggggtattttgatttcaacagattcc |
| | | | | actgccatttctcaccacgtgactggcagc |
| | | | | gactcatcaacaacaattggggattccgg |
| | | | | cccaagagactcaacttcaagctcttcaac |
| | | | | atccaagtcaaggaggtcacgacgaatg |
| | | | | atggcgtcacgaccatcgctaataacctta |
| | | | | ccagcacggttcaagtcttctcggactcg |
| | | | | gagtaccagttgccgtacgtcctcggctct |
| | | | | gcgcaccagggctgcctccctccgttccc |
| | | | | ggcggacgtgttcatgattccgcagtacg |
| | | | | gctacctaacgctcaacaatggcagccag |
| | | | | gcagtgggacggtcatccttttactgcctg |
| | | | | gaatatttcccatcgcagatgctgagaacg |
| | | | | ggcaataactttaccttcagctacaccttcg |
| | | | | aggacgtgcctttccacagcagctacgcg |
| | | | | cacagccagagcctggaccggctgatga |
| | | | | atcctctcatcgaccagtacctgtattacct |
| | | | | gaacagaactcagaatcagtccggaagt |
| | | | | gcccaaaacaaggacttgctgtttagccg |
| | | | | ggggtctccagctggcatgtctgttcagcc |
| | | | | caaaaactggctacctggaccctgttacc |
| | | | | ggcagcagcgcgtttctaaaacaaaaaca |
| | | | | gacaacaacaacagcaactttacctggac |
| | | | | tggtgcttcaaaatataaccttaatgggcgt |
| | | | | gaatctataatcaaccctggcactgctatg |
| | | | | gcctcacacaaagacgacaaagacaagt |
| | | | | tctttcccatgagcggtgtcatgatttttgga |
| | | | | aaggagagcgccggagcttcaaacactg |
| | | | | cattggacaatgtcatgatcacagacgaa |
| | | | | gaggaaatcaaagccactaaccccgtgg |
| | | | | ccaccgaaagatttgggactgtggcagtc |
| | | | | aatctccagagcagcagcacagaccctg |
| | | | | cgaccggagatgtgcatgttatgggagcc |
| | | | | ttacctggaatggtgtggcaagacagaga |
| | | | | cgtatacctgcagggtcctatttgggccaa |
| | | | | aattcctcacacggatggacactttcaccc |
| | | | | gtctcctctcatgggcggctttggacttaa |
| | | | | gcacccgcctcctcagatcctcatcaaaa |
| | | | | acacgcctgttcctgcgaatcctccggca |
| | | | | gagttttcggctacaaagtttgcttcattcat |
| | | | | cacccagtattccacaggacaagtgagc |
| | | | | gtggagattgaatgggagctgcagaaag |
| | | | | aaaacagcaaacgctggaatcccgaagt |
| | | | | gcagtatacatctaactatgcaaaatctgc |
| | | | | caacgttgatttcactgtggacaacaatgg |
| | | | | actttatactgagcctcgccccattggcac |
| | | | | ccgttacctcacccgtcccctgtaattgtgt |
| | | | | gttaatcaataaaccggt |
| Chimera 3b | | | 62 | ggtaccaaaacaaatgttctcgtcacgtgg |
| AAV4VP1/2- | | | | gcatgaatctgatgctgtttccctgcagac |
| AAV6VP3 | | | | aatgcgagagaatgaatcagaattcaaat |
| | | | | atctgcttcactcacggacagaaagactgt |
| | | | | ttagagtgctttcccgtgtcagaatctcaac |
| | | | | ccgtttctgtcgtcaaaaaggcgtatcaga |
| | | | | aactgtgctacattcatcatatcatgggaaa |
| | | | | ggtgccagacgcttgcactgcctgcgatc |
| | | | | tggtcaatgtggatttggatgactgcatcttt |
| | | | | gaacaataaatgatttaaatcaggtatgact |
| | | | | gacggttaccttccagattggctagagga |
| | | | | caacctctctgaaggcgttcgagagtggt |
| | | | | gggcgctgcaacctggagcccctaaacc |
| | | | | caaggcaaatcaacaacatcaggacaac |
| | | | | gctcggggtcttgtgcttccgggttacaaa |
| | | | | tacctcggacccggcaacggactcgaca |
| | | | | agggggaacccgtcaacgcagcggacg |
| | | | | cggcagccctcgagcacgacaaggccta |
| | | | | cgaccagcagctcaaggccggtgacaac |
| | | | | ccctacctcaagtacaaccacgccgacgc |
| | | | | ggagttccagcagcggcttcagggcgac |
| | | | | acatcgtttgggggcaacctcggcagag |
| | | | | cagtcttccaggccaaaaagagggttctt |
| | | | | gaacctcttggtctggttgagcaagcggg |
| | | | | tgagacggctcctggaaagaagagaccg |
| | | | | ttgattgaatccccccagcagcccgactc |
| | | | | ctccacgggtatcggcaaaaaaggcaag |
| | | | | cagccggctaaaaagaagctcgttttcga |
| | | | | agacgaaactggagcaggcgacggacc |
| | | | | ccctgagggatcaacttccggagccatgt |
| | | | | ctgatgacagtgagatggcttcaggcggt |
| | | | | ggcgcaccaatggcagacaataacgaag |
| | | | | gcgccgacggagtgggtaatgcctcagg |
| | | | | aaattggcattgcgattccacatggctggg |
| | | | | cgacagagtcatcaccaccagcacccga |
| | | | | acatgggccttgcccacctataacaacca |
| | | | | cctctacaagcaaatctccagtgcttcaac |
| | | | | gggggccagcaacgacaaccactacttc |
| | | | | ggctacagcaccccctgggggtattttgat |
| | | | | ttcaacagattccactgccatttctcaccac |
| | | | | gtgactggcagcgactcatcaacaacaat |
| | | | | tggggattccggcccaagagactcaactt |
| | | | | caagctcttcaacatccaagtcaaggagg |
| | | | | tcacgacgaatgatggcgtcacgaccatc |
| | | | | gctaataaccttaccagcacggttcaagtc |
| | | | | ttctcggactcggagtaccagttgccgtac |
| | | | | gtcctcggctctgcgcaccagggctgcct |
| | | | | ccctccgttcccggcggacgtgttcatgat |
| | | | | tccgcagtacggctacctaacgctcaaca |
| | | | | atggcagccaggcagtgggacggtcatc |
| | | | | cttttactgcctggaatatttcccatcgcag |
| | | | | atgctgagaacgggcaataactttaccttc |
| | | | | agctacaccttcgaggacgtgcctttccac |
| | | | | agcagctacgcgcacagccagagcctg |
| | | | | gaccggctgatgaatcctctcatcgacca |
| | | | | gtacctgtattacctgaacagaactcagaa |
| | | | | tcagtccggaagtgcccaaaacaaggac |
| | | | | ttgctgtttagccgggggtctccagctggc |
| | | | | atgtctgttcagcccaaaaactggctacct |
| | | | | ggaccctgttaccggcagcagcgcgtttc |
| | | | | taaaacaaaaacagacaacaacaacagc |
| | | | | aactttacctggactggtgcttcaaaatata |
| | | | | accttaatgggcgtgaatctataatcaacc |
| | | | | ctggcactgctatggcctcacacaaagac |
| | | | | gacaaagacaagttctttcccatgagcggt |
| | | | | gtcatgatttttggaaaggagagcgccgg |
| | | | | agcttcaaacactgcattggacaatgtcat |
| | | | | gatcacagacgaagaggaaatcaaagcc |
| | | | | actaaccccgtggccaccgaaagatttgg |
| | | | | gactgtggcagtcaatctccagagcagca |
| | | | | gcacagaccctgcgaccggagatgtgca |
| | | | | tgttatgggagccttacctggaatggtgtg |
| | | | | gcaagacagagacgtatacctgcagggt |
| | | | | cctatttgggccaaaattcctcacacggat |
| | | | | ggacactttcacccgtctcctctcatgggc |
| | | | | ggctaggacttaagcacccgcctcctcag |
| | | | | atcctcatcaaaaacacgcctgttcctgcg |
| | | | | aatcctccggcagagttttcggctacaaag |
| | | | | tttgcttcattcatcacccagtattccacag |
| | | | | gacaagtgagcgtggagattgaatggga |
| | | | | gctgcagaaagaaaacagcaaacgctgg |
| | | | | aatcccgaagtgcagtatacatctaactat |
| | | | | gcaaaatctgccaacgttgatttcactgtg |
| | | | | gacaacaatggactttatactgagcctcgc |
| | | | | cccattggcacccgttacctcacccgtccc |
| | | | | ctgtaattgtgtgttaatcaataaaccggt |
| Chimera 4b | | | 63 | ggtaccaaaacaaatgttctcgtcacgtgg |
| AAV5VP1_2- | | | | gcatgaatctgatgctgtttccctgcagac |
| AAV6VP3 | | | | aatgcgagagaatgaatcagaattcaaat |
| | | | | atctgcttcactcacggacagaaagactgt |
| | | | | ttagagtgctttcccgtgtcagaatctcaac |
| | | | | ccgtttctgtcgtcaaaaaggcgtatcaga |
| | | | | aactgtgctacattcatcatatcatgggaaa |
| | | | | ggtgccagacgcttgcactgcctgcgatc |
| | | | | tggtcaatgtggatttggatgactgcatcttt |
| | | | | gaacaataaatgatttaaatcaggtatgtct |
| | | | | tttgttgatcaccctccagattggttggaag |
| | | | | aagttggtgaaggtcttcgcgagtttttgg |
| | | | | gccttgaagcgggcccaccgaaaccaaa |
| | | | | acccaatcagcagcatcaagatcaagccc |
| | | | | gtggtcttgtgctgcctggttataactatctc |
| | | | | ggacccggaaacggtctcgatcgaggag |
| | | | | agcctgtcaacagggcagacgaggtcgc |
| | | | | gcgagagcacgacatctcgtacaacgag |
| | | | | cagcttgaggcgggagacaacccctacc |
| | | | | tcaagtacaaccacgcggacgccgagttt |
| | | | | caggagaagctcgccgacgacacatcct |
| | | | | tcgggggaaacctcggaaaggcagtcttt |
| | | | | caggccaagaaaagggttctcgaacctttt |
| | | | | ggcctggttgaagagggtgctaagacgg |
| | | | | cccctaccggaaagcggatagacgacca |
| | | | | ctttccaaaaagaaagaaggctcggacc |
| | | | | gaagaggactccaagccttccacctcgtc |
| | | | | agacgccgaagctggacccagcggatc |
| | | | | ccagcagctgcaaatcccagcccaacca |
| | | | | gcctcaagtttgggagctgatacaatggct |
| | | | | tcaggcggtggcgcaccaatggcagaca |
| | | | | ataacgaaggcgccgacggagtgggtaa |
| | | | | tgcctcaggaaattggcattgcgattccac |
| | | | | atggctgggcgacagagtcatcaccacc |
| | | | | agcacccgaacatgggccttgcccaccta |
| | | | | taacaaccacctctacaagcaaatctccag |
| | | | | tgcttcaacgggggccagcaacgacaac |
| | | | | cactacttcggctacagcaccccctgggg |
| | | | | gtattttgatttcaacagattccactgccattt |
| | | | | ctcaccacgtgactggcagcgactcatca |
| | | | | acaacaattggggattccggcccaagag |
| | | | | actcaacttcaagctcttcaacatccaagtc |
| | | | | aaggaggtcacgacgaatgatggcgtca |
| | | | | cgaccatcgctaataaccttaccagcacg |
| | | | | gttcaagtcttctcggactcggagtaccag |
| | | | | ttgccgtacgtcctcggctctgcgcacca |
| | | | | gggctgcctccctccgttcccggcggac |
| | | | | gtgttcatgattccgcagtacggctaccta |
| | | | | acgctcaacaatggcagccaggcagtgg |
| | | | | gacggtcatccttttactgcctggaatatttc |
| | | | | ccatcgcagatgctgagaacgggcaata |
| | | | | actttaccttcagctacaccttcgaggacgt |
| | | | | gcctttccacagcagctacgcgcacagcc |
| | | | | agagcctggaccggctgatgaatcctctc |
| | | | | atcgaccagtacctgtattacctgaacaga |
| | | | | actcagaatcagtccggaagtgcccaaaa |
| | | | | caaggacttgctgtttagccgggggtctcc |
| | | | | agctggcatgtctgttcagcccaaaaactg |
| | | | | gctacctggaccctgttaccggcagcagc |
| | | | | gcgtttctaaaacaaaaacagacaacaac |
| | | | | aacagcaactttacctggactggtgcttca |
| | | | | aaatataaccttaatgggcgtgaatctataa |
| | | | | tcaaccctggcactgctatggcctcacac |
| | | | | aaagacgacaaagacaagttctttcccat |
| | | | | gagcggtgtcatgatttttggaaaggaga |
| | | | | gcgccggagcttcaaacactgcattggac |
| | | | | aatgtcatgatcacagacgaagaggaaat |
| | | | | caaagccactaaccccgtggccaccgaa |
| | | | | agatttgggactgtggcagtcaatctccag |
| | | | | agcagcagcacagaccctgcgaccgga |
| | | | | gatgtgcatgttatgggagccttacctgga |
| | | | | atggtgtggcaagacagagacgtatacct |
| | | | | gcagggtcctatagggccaaaattcctca |
| | | | | cacggatggacactttcacccgtctcctct |
| | | | | catgggcggctaggacttaagcacccgc |
| | | | | ctcctcagatcctcatcaaaaacacgcctg |
| | | | | ttcctgcgaatcctccggcagagtatcgg |
| | | | | ctacaaagtagcttcattcatcacccagtat |
| | | | | tccacaggacaagtgagcgtggagattg |
| | | | | aatgggagctgcagaaagaaaacagcaa |
| | | | | acgctggaatcccgaagtgcagtatacat |
| | | | | ctaactatgcaaaatctgccaacgagata |
| | | | | cactgtggacaacaatggactttatactga |
| | | | | gcctcgccccattggcacccgttacctca |
| | | | | cccgtcccctgtaattgtgtgaaatcaata |
| | | | | aaccggt |
| Chimera 5b | | | 64 | ggtaccaaaacaaatgttctcgtcacgtgg |
| AAV11VP1/2- | | | | gcatgaatctgatgctgtaccctgcagac |
| AAV6VP3 | | | | aatgcgagagaatgaatcagaattcaaat |
| | | | | atctgcttcactcacggacagaaagactgt |
| | | | | ttagagtgctacccgtgtcagaatctcaac |
| | | | | ccgtactgtcgtcaaaaaggcgtatcaga |
| | | | | aactgtgctacattcatcatatcatgggaaa |
| | | | | ggtgccagacgcttgcactgcctgcgatc |
| | | | | tggtcaatgtggataggatgactgcatcta |
| | | | | gaacaataaatgatttaaatcaggtatggc |
| | | | | tgctgacggttatcttccagattggctcga |
| | | | | ggacaacctctctgagggcattcgcgagt |
| | | | | ggtgggacctgaaacctggagccccgaa |
| | | | | gcccaaggccaaccagcagaagcagga |
| | | | | cgacggccggggtctggtgcttcctggct |
| | | | | acaagtacctcggacccttcaacggactc |
| | | | | gacaagggggagcccgtcaacgcggcg |
| | | | | gacgcagcggccctcgagcacgacaag |
| | | | | gcctacgaccagcagctcaaagcgggtg |
| | | | | acaatccgtacctgcggtataaccacgcc |
| | | | | gacgccgagtttcaggagcgtctgcaag |
| | | | | aagatacgtcttagggggcaacctcggg |
| | | | | cgagcagtcttccaggccaagaagaggg |
| | | | | tactcgaacctctgggcctggttgaagaa |
| | | | | ggtgctaaaacggctcctggaaagaaga |
| | | | | gaccgttagagtcaccacaagagcccga |
| | | | | ctcctcctcgggcatcggcaaaaaaggc |
| | | | | aaacaaccagccagaaagaggctcaact |
| | | | | ttgaagaggacactggagccggagacg |
| | | | | gaccccctgaaggatcagataccagcgc |
| | | | | catgtcttcagacattgaaatggcttcagg |
| | | | | cggtggcgcaccaatggcagacaataac |
| | | | | gaaggcgccgacggagtgggtaatgcct |
| | | | | caggaaattggcattgcgattccacatgg |
| | | | | ctgggcgacagagtcatcaccaccagca |
| | | | | cccgaacatgggccttgcccacctataac |
| | | | | aaccacctctacaagcaaatctccagtgct |
| | | | | tcaacgggggccagcaacgacaaccact |
| | | | | acttcggctacagcaccccctgggggtat |
| | | | | tttgatttcaacagattccactgccatttctc |
| | | | | accacgtgactggcagcgactcatcaaca |
| | | | | acaattggggattccggcccaagagactc |
| | | | | aacttcaagctcttcaacatccaagtcaag |
| | | | | gaggtcacgacgaatgatggcgtcacga |
| | | | | ccatcgctaataaccttaccagcacggttc |
| | | | | aagtcttctcggactcggagtaccagttgc |
| | | | | cgtacgtcctcggctctgcgcaccagggc |
| | | | | tgcctccctccgttcccggcggacgtgttc |
| | | | | atgattccgcagtacggctacctaacgctc |
| | | | | aacaatggcagccaggcagtgggacggt |
| | | | | catccttttactgcctggaatatttcccatcg |
| | | | | cagatgctgagaacgggcaataactttac |
| | | | | cttcagctacaccttcgaggacgtgccttt |
| | | | | ccacagcagctacgcgcacagccagag |
| | | | | cctggaccggctgatgaatcctctcatcga |
| | | | | ccagtacctgtattacctgaacagaactca |
| | | | | gaatcagtccggaagtgcccaaaacaag |
| | | | | gacttgctgtttagccgggggtctccagct |
| | | | | ggcatgtctgttcagcccaaaaactggcta |
| | | | | cctggaccctgttaccggcagcagcgcgt |
| | | | | ttctaaaacaaaaacagacaacaacaaca |
| | | | | gcaactttacctggactggtgcttcaaaat |
| | | | | ataaccttaatgggcgtgaatctataatcaa |
| | | | | ccctggcactgctatggcctcacacaaag |
| | | | | acgacaaagacaagttctttcccatgagc |
| | | | | ggtgtcatgatttttggaaaggagagcgc |
| | | | | cggagcttcaaacactgcattggacaatgt |
| | | | | catgatcacagacgaagaggaaatcaaa |
| | | | | gccactaaccccgtggccaccgaaagatt |
| | | | | tgggactgtggcagtcaatctccagagca |
| | | | | gcagcacagaccctgcgaccggagatgt |
| | | | | gcatgttatgggagccttacctggaatggt |
| | | | | gtggcaagacagagacgtatacctgcag |
| | | | | ggtcctatttgggccaaaattcctcacacg |
| | | | | gatggacactttcacccgtctcctctcatg |
| | | | | ggcggctttggacttaagcacccgcctcc |
| | | | | tcagatcctcatcaaaaacacgcctgttcct |
| | | | | gcgaatcctccggcagagttttcggctac |
| | | | | aaagtttgcttcattcatcacccagtattcca |
| | | | | caggacaagtgagcgtggagattgaatg |
| | | | | ggagctgcagaaagaaaacagcaaacg |
| | | | | ctggaatcccgaagtgcagtatacatctaa |
| | | | | ctatgcaaaatctgccaacgttgatttcact |
| | | | | gtggacaacaatggactttatactgagcct |
| | | | | cgccccattggcacccgttacctcacccg |
| | | | | tcccctgtaattgtgtgttaatcaataaacc |
| | | | | ggt |
| Chimera 6b | | | 65 | ggtaccaaaacaaatgttctcgtcacgtgg |
| AAV12VP1/2- | | | | gcatgaatctgatgctgtttccctgcagac |
| AAV6VP3 | | | | aatgcgagagaatgaatcagaattcaaat |
| | | | | atctgcttcactcacggacagaaagactgt |
| | | | | ttagagtgctttcccgtgtcagaatctcaac |
| | | | | ccgtttctgtcgtcaaaaaggcgtatcaga |
| | | | | aactgtgctacattcatcatatcatgggaaa |
| | | | | ggtgccagacgcttgcactgcctgcgatc |
| | | | | tggtcaatgtggatttggatgactgcatcttt |
| | | | | gaacaataaatgatttaaatcaggtatggc |
| | | | | tgctgacggttatcttccagattggctcga |
| | | | | ggacaacctctctgaaggcattcgcgagt |
| | | | | ggtgggcgctgaaacctggagctccaca |
| | | | | acccaaggccaaccaacagcatcaggac |
| | | | | aacggcaggggtcttgtgcttcctgggta |
| | | | | caagtacctcggacccttcaacggactcg |
| | | | | acaagggagagccggtcaacgaggcag |
| | | | | acgccgcggccctcgagcacgacaagg |
| | | | | cctacgacaagcagctcgagcaggggg |
| | | | | acaacccgtatctcaagtacaaccacgcc |
| | | | | gacgccgagttccagcagcgcttggcga |
| | | | | ccgacacctcttttgggggcaacctcggg |
| | | | | cgagcagtcttccaggccaaaaagagga |
| | | | | ttctcgagcctctgggtctggttgaagagg |
| | | | | gcgttaaaacggctcctggaaagaaacg |
| | | | | cccattagaaaagactccaaatcggccga |
| | | | | ccaacccggactctgggaaggccccgg |
| | | | | ccaagaaaaagcaaaaagacggcgaac |
| | | | | cagccgactctgctagaaggacactcga |
| | | | | ctttgaagactctggagcaggagacgga |
| | | | | ccccctgagggatcatcttccggagaaat |
| | | | | gtctcatgatgctgagatggcttcaggcg |
| | | | | gtggcgcaccaatggcagacaataacga |
| | | | | aggcgccgacggagtgggtaatgcctca |
| | | | | ggaaattggcattgcgattccacatggctg |
| | | | | ggcgacagagtcatcaccaccagcaccc |
| | | | | gaacatgggccttgcccacctataacaac |
| | | | | cacctctacaagcaaatctccagtgcttca |
| | | | | acgggggccagcaacgacaaccactact |
| | | | | tcggctacagcaccccctgggggtattttg |
| | | | | atttcaacagattccactgccatttctcacc |
| | | | | acgtgactggcagcgactcatcaacaaca |
| | | | | attggggattccggcccaagagactcaac |
| | | | | ttcaagctcttcaacatccaagtcaaggag |
| | | | | gtcacgacgaatgatggcgtcacgaccat |
| | | | | cgctaataaccttaccagcacggttcaagt |
| | | | | cttctcggactcggagtaccagttgccgta |
| | | | | cgtcctcggctctgcgcaccagggctgcc |
| | | | | tccctccgttcccggcggacgtgttcatga |
| | | | | ttccgcagtacggctacctaacgctcaac |
| | | | | aatggcagccaggcagtgggacggtcat |
| | | | | ccttttactgcctggaatatacccatcgca |
| | | | | gatgctgagaacgggcaataactttacctt |
| | | | | cagctacaccttcgaggacgtgcctacca |
| | | | | cagcagctacgcgcacagccagagcctg |
| | | | | gaccggctgatgaatcctctcatcgacca |
| | | | | gtacctgtattacctgaacagaactcagaa |
| | | | | tcagtccggaagtgcccaaaacaaggac |
| | | | | ttgctgatagccgggggtctccagctggc |
| | | | | atgtctgttcagcccaaaaactggctacct |
| | | | | ggaccctgttaccggcagcagcgcgtac |
| | | | | taaaacaaaaacagacaacaacaacagc |
| | | | | aactttacctggactggtgcttcaaaatata |
| | | | | accttaatgggcgtgaatctataatcaacc |
| | | | | ctggcactgctatggcctcacacaaagac |
| | | | | gacaaagacaagttctacccatgagcggt |
| | | | | gtcatgatattggaaaggagagcgccgg |
| | | | | agcttcaaacactgcattggacaatgtcat |
| | | | | gatcacagacgaagaggaaatcaaagcc |
| | | | | actaaccccgtggccaccgaaagatttgg |
| | | | | gactgtggcagtcaatctccagagcagca |
| | | | | gcacagaccctgcgaccggagatgtgca |
| | | | | tgttatgggagccttacctggaatggtgtg |
| | | | | gcaagacagagacgtatacctgcagggt |
| | | | | cctatttgggccaaaattcctcacacggat |
| | | | | ggacactacacccgtctcctctcatgggc |
| | | | | ggctttggacttaagcacccgcctcctcag |
| | | | | atcctcatcaaaaacacgcctgttcctgcg |
| | | | | aatcctccggcagagtatcggctacaaag |
| | | | | tagcttcattcatcacccagtattccacag |
| | | | | gacaagtgagcgtggagattgaatggga |
| | | | | gctgcagaaagaaaacagcaaacgctgg |
| | | | | aatcccgaagtgcagtatacatctaactat |
| | | | | gcaaaatctgccaacgttgatttcactgtg |
| | | | | gacaacaatggactttatactgagcctcgc |
| | | | | cccattggcacccgttacctcacccgtccc |
| | | | | ctgtaattgtgtgaaatcaataaaccggt |

TABLE 2
WT AAV capsid amino acid and nucleic acid sequences

| Virus | SEQ | | SEQ | |
|---|---|---|---|---|
| Serotype | ID NO. | Amino acid sequence | ID NO. | Nucleic acid sequence |
| AAV6 | 26 | MAADGYLPDWLEDNLSE | 31 | atggctgccgatggttatcttccagattgg |
| | | GIREWWDLKPGAPKPKA | | ctcgaggacaacctctctgagggcattcg |
| | | NQQKQDDGRGLVLPGYK | | cgagtggtgggacttgaaacctggagcc |
| | | YLGPFNGLDKGEPVNAAD | | ccgaaacccaaagccaaccagcaaaag |
| | | AAALEHDKAYDQQLKAG | | caggacgacggccggggtctggtgcttc |
| | | DNPYLRYNHADAEFQERL | | ctggctacaagtacctcggacccttcaac |
| | | QEDTSFGGNLGRAVFQAK | | ggactcgacaagggggagcccgtcaac |
| | | KRVLEPFGLVEEGAKTAP | | gcggcggatgcagcggccctcgagcac |
| | | GKKRPVEQSPQEPDSSSGI | | gacaaggcctacgaccagcagctcaaag |
| | | GKTGQQPAKKRLNFGQT | | cgggtgacaatccgtacctgcggtataac |
| | | GDSESVPDPQPLGEPPATP | | cacgccgacgccgagtttcaggagcgtct |
| | | AAVGPTTMASGGGAPMA | | gcaagaagatacgtcttttgggggcaacc |
| | | DNNEGADGVGNASGNWH | | tcgggcgagcagtcttccaggccaagaa |
| | | CDSTWLGDRVITTSTRTW | | gagggttctcgaaccttttggtctggttgag |
| | | ALPTYNNHLYKQISSASTG | | gaaggtgctaagacggctcctggaaaga |
| | | ASNDNHYFGYSTPWGYF | | aacgtccggtagagcagtcgccacaaga |
| | | DFNRFHCHFSPRDWQRLI | | gccagactcctcctcgggcattggcaaga |
| | | NNNWGFRPKRLNFKLFNI | | caggccagcagcccgctaaaaagagact |
| | | QVKEVTTNDGVTTIANNL | | caattttggtcagactggcgactcagagtc |
| | | TSTVQVFSDSEYQLPYVL | | agtccccgacccacaacctctcggagaa |
| | | GSAHQGCLPPFPADVFMIP | | cctccagcaacccccgctgctgtgggac |
| | | QYGYLTLNNGSQAVGRSS | | ctactacaatggcttcaggcggtggcgca |
| | | FYCLEYFPSQMLRTGNNF | | ccaatggcagacaataacgaaggcgccg |
| | | TFSYTFEDVPFHSSYAHSQ | | acggagtgggtaatgcctcaggaaattgg |
| | | SLDRLMNPLIDQYLYYLN | | cattgcgattccacatggctgggcgacag |
| | | RTQNQSGSAQNKDLLFSR | | agtcatcaccaccagcacccgaacatgg |
| | | GSPAGMSVQPKNWLPGP | | gccttgcccacctataacaaccacctctac |
| | | CYRQQRVSKTKTDNNNS | | aagcaaatctccagtgcttcaacgggggc |
| | | NFTWTGASKYNLNGRESII | | cagcaacgacaaccactacttcggctaca |
| | | NPGTAMASHKDDKDKFFP | | gcaccccctgggggtattttgatttcaaca |
| | | MSGVMIFGKESAGASNTA | | gattccactgccatttctcaccacgtgactg |
| | | LDNVMITDEEEIKATNPV | | gcagcgactcatcaacaacaattggggat |
| | | ATERFGTVAVNLQSSSTD | | tccggcccaagagactcaacttcaagctc |
| | | PATGDVHVMGALPGMV | | ttcaacatccaagtcaaggaggtcacgac |
| | | WQDRDVYLQGPIWAKIPH | | gaatgatggcgtcacgaccatcgctaata |
| | | TDGHFHPSPLMGGFGLKH | | accttaccagcacggttcaagtcttctcgg |
| | | PPPQILIKNTPVPANPPAEF | | actcggagtaccagttgccgtacgtcctc |
| | | SATKFASFITQYSTGQVSV | | ggctctgcgcaccagggctgcctccctcc |
| | | EIEWELQKENSKRWNPEV | | gttcccggcggacgtgttcatgattccgca |
| | | QYTSNYAKSANVDFTVD | | gtacggctacctaacgctcaacaatggca |
| | | NNGLYTEPRPIGTRYLTRP | | gccaggcagtgggacggtcatccttttact |
| | | L | | gcctggaatatttcccatcgcagatgctga |
| | | | | gaacgggcaataactttaccttcagctaca |
| | | | | ccttcgaggacgtgcctttccacagcagct |
| | | | | acgcgcacagccagagcctggaccggc |
| | | | | tgatgaatcctctcatcgaccagtacctgta |
| | | | | ttacctgaacagaactcagaatcagtccg |
| | | | | gaagtgcccaaaacaaggacttgctgttta |
| | | | | gccgggggtctccagctggcatgtctgtt |
| | | | | cagcccaaaaactggctacctggaccctg |
| | | | | ttaccggcagcagcgcgtttctaaaacaa |
| | | | | aaacagacaacaacaacagcaactttacc |
| | | | | tggactggtgcttcaaaatataaccttaatg |
| | | | | ggcgtgaatctataatcaaccctggcactg |
| | | | | ctatggcctcacacaaagacgacaaaga |
| | | | | caagttctttcccatgagcggtgtcatgatt |
| | | | | tttggaaaggagagcgccggagcttcaa |
| | | | | acactgcattggacaatgtcatgatcacag |
| | | | | acgaagaggaaatcaaagccactaaccc |
| | | | | cgtggccaccgaaagatttgggactgtgg |
| | | | | cagtcaatctccagagcagcagcacaga |
| | | | | ccctgcgaccggagatgtgcatgttatgg |
| | | | | gagccttacctggaatggtgtggcaagac |
| | | | | agagacgtatacctgcagggtcctatttgg |
| | | | | gccaaaattcctcacacggatggacacttt |
| | | | | cacccgtctcctctcatgggcggctttgga |
| | | | | cttaagcacccgcctcctcagatcctcatc |
| | | | | aaaaacacgcctgttcctgcgaatcctcc |
| | | | | ggcagagttttcggctacaaagtttgcttca |
| | | | | ttcatcacccagtattccacaggacaagtg |
| | | | | agcgtggagattgaatgggagctgcaga |
| | | | | aagaaaacagcaaacgctggaatcccga |
| | | | | agtgcagtatacatctaactatgcaaaatct |
| | | | | gccaacgttgatttcactgtggacaacaat |
| | | | | ggactttatactgagcctcgccccattggc |
| | | | | acccgttacctcacccgtcccctgtaa |
| AAV4 | 27 | MTDGYLPDWLEDNLSEG | 32 | atgactgacggttaccttccagattggcta |
| | | VREWWALQPGAPKPKAN | | gaggacaacctctctgaaggcgttcgaga |
| | | QQHQDNARGLVLPGYKY | | gtggtgggcgctgcaacctggagcccct |
| | | LGPGNGLDKGEPVNAAD | | aaacccaaggcaaatcaacaacatcagg |
| | | AAALEHDKAYDQQLKAG | | acaacgctcggggtcttgtgcttccgggtt |
| | | DNPYLKYNHADAEFQQR | | acaaatacctcggacccggcaacggact |
| | | LQGDTSFGGNLGRAVFQA | | cgacaagggggaacccgtcaacgcagc |
| | | KKRVLEPLGLVEQAGETA | | ggacgcggcagccctcgagcacgacaa |
| | | PGKKRPLIESPQQPDSSTGI | | ggcctacgaccagcagctcaaggccggt |
| | | GKKGKQPAKKKLVFEDET | | gacaacccctacctcaagtacaaccacgc |
| | | GAGDGPPEGSTSGAMSDD | | cgacgcggagttccagcagcggcttcag |
| | | SEMRAAAGGAAVEGGQG | | ggcgacacatcgtttgggggcaacctcg |
| | | ADGVGNASGDWHCDSTW | | gcagagcagtcttccaggccaaaaagag |
| | | SEGHVTTTSTRTWVLPTY | | ggttcttgaacctcttggtctggttgagcaa |
| | | NNHLYKRLGESLQSNTYN | | gcgggtgagacggctcctggaaagaag |
| | | GFSTPWGYFDFNRFHCHF | | agaccgttgattgaatccccccagcagcc |
| | | SPRDWQRLINNNWGMRP | | cgactcctccacgggtatcggcaaaaaa |
| | | KAMRVKIFNIQVKEVTTS | | ggcaagcagccggctaaaaagaagctc |
| | | NGETTVANNLTSTVQIFA | | glatcgaagacgaaactggagcaggcg |
| | | DS SYELPYVMDAGQEGSL | | acggaccccctgagggatcaacttccgg |
| | | PPFPNDVFMVPQYGYCGL | | agccatgtctgatgacagtgagatgcgtg |
| | | VTGNTSQQQTDRNAFYCL | | cagcagctggcggagctgcagtcgagg |
| | | EYFPSQMLRTGNNFEITYS | | gcggacaaggtgccgatggagtgggtaa |
| | | FEKVPFHSMYAHSQSLDR | | tgcctcgggtgattggcattgcgattccac |
| | | LMNPLIDQYLWGLQSTTT | | ctggtctgagggccacgtcacgaccacc |
| | | GTTLNAGTATTNFTKLRP | | agcaccagaacctgggtcttgcccaccta |
| | | TNFSNFKKNVVLPGPSIKQ | | caacaaccacctctacaagcgactcgga |
| | | QGFSKTANQNYKIPATGS | | gagagcctgcagtccaacacctacaacg |
| | | DSLIKYETHSTLDGRWSA | | gattctccaccccctggggatactttgactt |
| | | LTPGPPMATAGPADSKFS | | caaccgcttccactgccacttctcaccacg |
| | | NSQLIFAGPKQNGNTATV | | tgactggcagcgactcatcaacaacaact |
| | | PGTLIFTSEEELAATNATD | | ggggcatgcgacccaaagccatgcgggt |
| | | TDMWGNLPGGDQSNSNL | | caaaatcttcaacatccaggtcaaggagg |
| | | PTVDRLTALGAVPGMVW | | tcacgacgtcgaacggcgagacaacggt |
| | | QNRDIYYQGPIWAKIPHT | | ggctaataaccttaccagcacggttcagat |
| | | DGHFHPSPLIGGFGLKHPP | | ctttgcggactcgtcgtacgaactgccgta |
| | | PQIFIKNTPVPANPATTFSS | | cgtgatggatgcgggtcaagagggcagc |
| | | TPVNSFITQYSTGQVSVQI | | ctgcctccttttcccaacgacgtctttatggt |
| | | DWEIQKERSKRWNPEVQF | | gccccagtacggctactgtggactggtga |
| | | TSNYGQQNSLLWAPDAA | | ccggcaacacttcgcagcaacagactga |
| | | GKYTEPRAIGTRYLTHHL | | cagaaatgccttctactgcctggagtacttt |
| | | | | ccttcgcagatgctgcggactggcaacaa |
| | | | | ctttgaaattacgtacagttttgagaaggtg |
| | | | | cctttccactcgatgtacgcgcacagcca |
| | | | | gagcctggaccggctgatgaaccctctca |
| | | | | tcgaccagtacctgtggggactgcaatcg |
| | | | | accaccaccggaaccaccctgaatgccg |
| | | | | ggactgccaccaccaactttaccaagctg |
| | | | | cggcctaccaacttttccaactttaaaaaga |
| | | | | actggctgcccgggccttcaatcaagcag |
| | | | | cagggcttctcaaagactgccaatcaaaa |
| | | | | ctacaagatccctgccaccgggtcagaca |
| | | | | gtctcatcaaatacgagacgcacagcact |
| | | | | ctggacggaagatggagtgccctgaccc |
| | | | | ccggacctccaatggccacggctggacc |
| | | | | tgcggacagcaagttcagcaacagccag |
| | | | | ctcatctttgcggggcctaaacagaacgg |
| | | | | caacacggccaccgtacccgggactctg |
| | | | | atcttcacctctgaggaggagctggcagc |
| | | | | caccaacgccaccgatacggacatgtgg |
| | | | | ggcaacctacctggcggtgaccagagca |
| | | | | acagcaacctgccgaccgtggacagact |
| | | | | gacagccttgggagccgtgcctggaatg |
| | | | | gtctggcaaaacagagacatttactacca |
| | | | | gggtcccatttgggccaagattcctcatac |
| | | | | cgatggacactttcacccctcaccgctgat |
| | | | | tggtgggtttgggctgaaacacccgcctc |
| | | | | ctcaaatttttatcaagaacaccccggtacc |
| | | | | tgcgaatcctgcaacgaccttcagctctac |
| | | | | tccggtaaactccttcattactcagtacagc |
| | | | | actggccaggtgtcggtgcagattgactg |
| | | | | ggagatccagaaggagcggtccaaacg |
| | | | | ctggaaccccgaggtccagtttacctcca |
| | | | | actacggacagcaaaactctctgttgtgg |
| | | | | gctcccgatgcggctgggaaatacactga |
| | | | | gcctagggctatcggtacccgctacctca |
| | | | | cccaccacctgtaa |
| AAV5 | 28 | MSFVDHPPDWLEEVGEGL | 33 | atgtcttttgttgatcaccctccagattggtt |
| | | REFLGLEAGPPKPKPNQQ | | ggaagaagttggtgaaggtcttcgcgagt |
| | | HQDQARGLVLPGYNYLG | | ttttgggccttgaagcgggcccaccgaaa |
| | | PGNGLDRGEPVNRADEVA | | ccaaaacccaatcagcagcatcaagatca |
| | | REHDISYNEQLEAGDNPY | | agcccgtggtcttgtgctgcctggttataa |
| | | LKYNHADAEFQEKLADD | | ctatctcggacccggaaacggtctcgatc |
| | | TSFGGNLGKAVFQAKKRV | | gaggagagcctgtcaacagggcagacg |
| | | LEPFGLVEEGAKTAPTGK | | aggtcgcgcgagagcacgacatctcgta |
| | | RIDDHFPKRKKARTEEDS | | caacgagcagcttgaggcgggagacaa |
| | | KPSTSSDAEAGPSGSQQL | | cccctacctcaagtacaaccacgcggac |
| | | QIPAQPASSLGADTMSAG | | gccgagtttcaggagaagctcgccgacg |
| | | GGGPLGDNNQGADGVGN | | acacatccttcgggggaaacctcggaaa |
| | | ASGDWHCDSTWMGDRV | | ggcagtctttcaggccaagaaaagggttc |
| | | VTKSTRTWVLPSYNNHQY | | tcgaaccttttggcctggttgaagagggtg |
| | | REIKSGSVDGSNANAYFG | | ctaagacggcccctaccggaaagcggat |
| | | YSTPWGYFDFNRFHSHWS | | agacgaccactttccaaaaagaaagaag |
| | | PRDWQRLINNYWGFRPRS | | gctcggaccgaagaggactccaagcctt |
| | | LRVKIFNIQVKEVTVQDST | | ccacctcgtcagacgccgaagctggacc |
| | | TTIANNLTSTVQVFTDDD | | cagcggatcccagcagctgcaaatccca |
| | | YQLPYVVGNGTEGCLPAF | | gcccaaccagcctcaagtttgggagctga |
| | | PPQVFTLPQYGYATLNRD | | tacaatgtctgcgggaggtggcggcccat |
| | | NTENPTERSSFFCLEYFPS | | tgggcgacaataaccaaggtgccgatgg |
| | | KMLRTGNNFEFTYNFEEV | | agtgggcaatgcctcgggagattggcatt |
| | | PFHSSFAPSQNLFKLANPL | | gcgattccacgtggatgggggacagagt |
| | | VDQYLYRFVSTNNTGGV | | cgtcaccaagtccacccgaacctgggtg |
| | | QFNKNLAGRYANTYKNW | | ctgcccagctacaacaaccaccagtaccg |
| | | FPGPMGRTQGWNLGSGV | | agagatcaaaagcggctccgtcgacgga |
| | | NRASVSAFATTNRMELEG | | agcaacgccaacgcctactttggatacag |
| | | ASYQVPPQPNGMTNNLQ | | caccccctgggggtactttgactttaaccg |
| | | GSNTYALENTMIFNSQPA | | cttccacagccactggagcccccgagact |
| | | NPGTTATYLEGNMLITSES | | ggcaaagactcatcaacaactactgggg |
| | | ETQPVNRVAYNVGGQMA | | cttcagaccccggtccctcagagtcaaaa |
| | | TNNQSSTTAPATGTYNLQ | | tcttcaacattcaagtcaaagaggtcacgg |
| | | EIVPGSVWMERDVYLQGP | | tgcaggactccaccaccaccatcgccaac |
| | | IWAKIPETGAHFHPSPAM | | aacctcacctccaccgtccaagtgtttacg |
| | | GGFGLKHPPPMMLIKNTP | | gacgacgactaccagctgccctacgtcgt |
| | | VPGNITSFSDVPVSSFITQY | | cggcaacgggaccgagggatgcctgcc |
| | | STGQVTVEMEWELKKEN | | ggccttccctccgcaggtctttacgctgcc |
| | | SKRWNPEIQYTNNYNDPQ | | gcagtacggttacgcgacgctgaaccgc |
| | | FVDFAPDSTGEYRTTRPIG | | gacaacacagaaaatcccaccgagagga |
| | | TRYLTRPL | | gcagcttcttctgcctagagtactttcccag |
| | | | | caagatgctgagaacgggcaacaactttg |
| | | | | agtttacctacaactttgaggaggtgccctt |
| | | | | ccactccagcttcgctcccagtcagaacct |
| | | | | gttcaagctggccaacccgctggtggacc |
| | | | | agtacttgtaccgcttcgtgagcacaaata |
| | | | | acactggcggagtccagttcaacaagaa |
| | | | | cctggccgggagatacgccaacacctac |
| | | | | aaaaactggttcccggggcccatgggcc |
| | | | | gaacccagggctggaacctgggctccgg |
| | | | | ggtcaaccgcgccagtgtcagcgccttcg |
| | | | | ccacgaccaataggatggagctcgaggg |
| | | | | cgcgagttaccaggtgcccccgcagccg |
| | | | | aacggcatgaccaacaacctccagggca |
| | | | | gcaacacctatgccctggagaacactatg |
| | | | | atcttcaacagccagccggcgaacccgg |
| | | | | gcaccaccgccacgtacctcgagggcaa |
| | | | | catgctcatcaccagcgagagcgagacg |
| | | | | cagccggtgaaccgcgtggcgtacaacg |
| | | | | tcggcgggcagatggccaccaacaacca |
| | | | | gagctccaccactgcccccgcgaccggc |
| | | | | acgtacaacctccaggaaatcgtgcccg |
| | | | | gcagcgtgtggatggagagggacgtgta |
| | | | | cctccaaggacccatctgggccaagatcc |
| | | | | cagagacgggggcgcactttcacccctct |
| | | | | ccggccatgggcggattcggactcaaac |
| | | | | acccaccgcccatgatgctcatcaagaac |
| | | | | acgcctgtgcccggaaatatcaccagctt |
| | | | | ctcggacgtgcccgtcagcagcttcatca |
| | | | | cccagtacagcaccgggcaggtcaccgt |
| | | | | ggagatggagtgggagctcaagaagga |
| | | | | aaactccaagaggtggaacccagagatc |
| | | | | cagtacacaaacaactacaacgaccccc |
| | | | | agtttgtggactttgccccggacagcacc |
| | | | | ggggaatacagaaccaccagacctatcg |
| | | | | gaacccgataccttacccgacccctttaa |
| AAV11 | 29 | MAADGYLPDWLEDNLSE | 34 | atggctgctgacggttatcttccagattgg |
| | | GIREWWDLKPGAPKPKA | | ctcgaggacaacctctctgagggcattcg |
| | | NQQKQDDGRGLVLPGYK | | cgagtggtgggacctgaaacctggagcc |
| | | YLGPFNGLDKGEPVNAAD | | ccgaagcccaaggccaaccagcagaag |
| | | AAALEHDKAYDQQLKAG | | caggacgacggccggggtctggtgcttc |
| | | DNPYLRYNHADAEFQERL | | ctggctacaagtacctcggacccttcaac |
| | | QEDTSFGGNLGRAVFQAK | | ggactcgacaagggggagcccgtcaac |
| | | KRVLEPLGLVEEGAKTAP | | gcggcggacgcagcggccctcgagcac |
| | | GKKRPLESPQEPDSSSGIG | | gacaaggcctacgaccagcagctcaaag |
| | | KKGKQPARKRLNFEEDTG | | cgggtgacaatccgtacctgcggtataac |
| | | AGDGPPEGSDTSAMSSDIE | | cacgccgacgccgagtttcaggagcgtct |
| | | MRAAPGGNAVDAGQGSD | | gcaagaagatacgtcttttgggggcaacc |
| | | GVGNASGDWHCDSTWSE | | tcgggcgagcagtcttccaggccaagaa |
| | | GKVTTTSTRTWVLPTYNN | | gagggtactcgaacctctgggcctggttg |
| | | HLYLRLGTTSSSNTYNGFS | | aagaaggtgctaaaacggctcctggaaa |
| | | TPWGYFDFNRFHCHFSPR | | gaagagaccgttagagtcaccacaagag |
| | | DWQRLINNNWGLRPKAM | | cccgactcctcctcgggcatcggcaaaaa |
| | | RVKIFNIQVKEVTTSNGET | | aggcaaacaaccagccagaaagaggct |
| | | TVANNLTSTVQIFADSSYE | | caactttgaagaggacactggagccgga |
| | | LPYVMDAGQEGSLPPFPN | | gacggaccccctgaaggatcagatacca |
| | | DVFMVPQYGYCGIVTGEN | | gcgccatgtcttcagacattgaaatgcgtg |
| | | QNQTDRNAFYCLEYFPSQ | | cagcaccgggcggaaatgctgtcgatgc |
| | | MLRTGNNFECANNFEKVP | | gggacaaggttccgatggagtgggtaat |
| | | FHSMYAHSQSLDRLMNPL | | gcctcgggtgattggcattgcgattccacc |
| | | LDQYLWHLQSTTSGETLN | | tggtctgagggcaaggtcacaacaacctc |
| | | QGNAATTFGKIRSGDFAF | | gaccagaacctgggtcttgcccacctaca |
| | | YRKNWLPGPCVKQQRFS | | acaaccacttgtacctgcgtctcggaaca |
| | | KTASQNYKIPASGGNALL | | acatcaagcagcaacacctacaacggatt |
| | | KYDTHYTLNNRWSNIAPG | | ctccaccccctggggatattttgacttcaac |
| | | PPMATAGPSDGDFSNAQL | | agattccactgtcacttctcaccacgtgact |
| | | IFPGPSVTGNTTTSANNLL | | ggcaaagactcatcaacaacaactgggg |
| | | FTSEEEIAATNPRDTDMFG | | actacgaccaaaagccatgcgcgttaaaa |
| | | QIADNNQNATTAPITGNV | | tcttcaatatccaagttaaggaggtcacaa |
| | | TAMGVLPGMVWQNRDIY | | cgtcgaacggcgagactacggtcgctaat |
| | | YQGPIWAKIPHADGHFHP | | aaccttaccagcacggttcagatatttgcg |
| | | SPLIGGFGLKHPPPQIFIKN | | gactcgtcgtatgagctcccgtacgtgatg |
| | | TPVPANPATTFTAARVDSF | | gacgctggacaagaggggagcctgcctc |
| | | ITQYSTGQVAVQIEWEIEK | | ctttccccaatgacgtgttcatggtgcctca |
| | | ERSKRWNPEVQFTSNYGN | | atatggctactgtggcatcgtgactggcga |
| | | QSSMLWAPDTTGKYTEPR | | gaatcagaaccaaacggacagaaacgct |
| | | VIGSRYLTNHL | | ttctactgcctggagtattttccttcgcaaat |
| | | | | gttgagaactggcaacaactttgaaatgg |
| | | | | cttacaactttgagaaggtgccgttccact |
| | | | | caatgtatgctcacagccagagcctggac |
| | | | | agactgatgaatcccctcctggaccagta |
| | | | | cctgtggcacttacagtcgactacctctgg |
| | | | | agagactctgaatcaaggcaatgcagca |
| | | | | accacatttggaaaaatcaggagtggaga |
| | | | | ctttgccttttacagaaagaactggctgcct |
| | | | | gggccttgtgttaaacagcagagattctca |
| | | | | aaaactgccagtcaaaattacaagattcct |
| | | | | gccagcgggggcaacgctctgttaaagt |
| | | | | atgacacccactataccttaaacaaccgct |
| | | | | ggagcaacatcgcgcccggacctccaat |
| | | | | ggccacagccggaccttcggatggggac |
| | | | | ttcagtaacgcccagcttatattccctggac |
| | | | | catctgttaccggaaatacaacaacttcag |
| | | | | ccaacaatctgttgtttacatcagaagaag |
| | | | | aaattgctgccaccaacccaagagacac |
| | | | | ggacatgtttggccagattgctgacaataa |
| | | | | tcagaatgctacaactgctcccataaccg |
| | | | | gcaacgtgactgctatgggagtgctgcct |
| | | | | ggcatggtgtggcaaaacagagacattta |
| | | | | ctaccaagggccaatttgggccaagatcc |
| | | | | cacacgcggacggacattttcatccttcac |
| | | | | cgctgattggtgggtttggactgaaacacc |
| | | | | cgcctccccagatattcatcaagaacactc |
| | | | | ccgtacctgccaatcctgcgacaaccttca |
| | | | | ctgcagccagagtggactctttcatcacac |
| | | | | aatacagcaccggccaggtcgctgttcag |
| | | | | attgaatgggaaattgaaaaggaacgctc |
| | | | | caaacgctggaatcctgaagtgcagtttac |
| | | | | ttcaaactatgggaaccagtcttctatgttgt |
| | | | | gggctcctgatacaactgggaagtataca |
| | | | | gagccgcgggttattggctctcgttatttga |
| | | | | ctaatcatttgtaa |
| AAV12 | 30 | MAADGYLPDWLEDNLSE | 35 | atggctgctgacggttatcttccagattgg |
| | | GIREWWALKPGAPQPKA | | ctcgaggacaacctctctgaaggcattcg |
| | | NQQHQDNGRGLVLPGYK | | cgagtggtgggcgctgaaacctggagct |
| | | YLGPFNGLDKGEPVNEAD | | ccacaacccaaggccaaccaacagcatc |
| | | AAALEHDKAYDKQLEQG | | aggacaacggcaggggtcttgtgcttcct |
| | | DNPYLKYNHADAEFQQR | | gggtacaagtacctcggacccttcaacgg |
| | | LATDTSFGGNLGRAVFQA | | actcgacaagggagagccggtcaacga |
| | | KKRILEPLGLVEEGVKTAP | | ggcagacgccgcggccctcgagcacga |
| | | GKKRPLEKTPNRPTNPDS | | caaggcctacgacaagcagctcgagcag |
| | | GKAPAKKKQKDGEPADS | | ggggacaacccgtatctcaagtacaacca |
| | | ARRTLDFEDSGAGDGPPE | | cgccgacgccgagttccagcagcgcttg |
| | | GSSSGEMSHDAEMRAAP | | gcgaccgacacctcttttgggggcaacct |
| | | GGNAVEAGQGADGVGNA | | cgggcgagcagtcttccaggccaaaaag |
| | | SGDWHCDSTWSEGRVTT | | aggattctcgagcctctgggtctggttgaa |
| | | TSTRTWVLPTYNNHLYLR | | gagggcgttaaaacggctcctggaaaga |
| | | IGTTANSNTYNGFSTPWG | | aacgcccattagaaaagactccaaatcgg |
| | | YFDFNRFHCHFSPRDWQR | | ccgaccaacccggactctgggaaggccc |
| | | LINNNWGLRPKSMRVKIF | | cggccaagaaaaagcaaaaagacggcg |
| | | NIQVKEVTTSNGETTVAN | | aaccagccgactctgctagaaggacactc |
| | | NLTSTVQIFADSTYELPYV | | gactttgaagactctggagcaggagacg |
| | | MDAGQEGSFPPFPNDVFM | | gaccccctgagggatcatcttccggagaa |
| | | VPQYGYCGVVTGKNQNQ | | atgtctcatgatgctgagatgcgtgcggc |
| | | TDRNAFYCLEYFPSQMLR | | gccaggcggaaatgctgtcgaggcggg |
| | | TGNNFEVSYQFEKVPFHS | | acaaggtgccgatggagtgggtaatgcct |
| | | MYAHSQSLDRMMNPLLD | | ccggtgattggcattgcgattccacctggt |
| | | QYLWHLQSTTTGNSLNQG | | cagagggccgagtcaccaccaccagca |
| | | TATTTYGKITTGDFAYYR | | cccgaacctgggtcctacccacgtacaac |
| | | KNWLPGACIKQQKFSKNA | | aaccacctgtacctgcgaatcggaacaac |
| | | NQNYKIPASGGDALLKYD | | ggccaacagcaacacctacaacggattct |
| | | THTTLNGRWSNMAPGPP | | ccaccccctggggatactttgactttaacc |
| | | MATAGAGDSDFSNSQLIF | | gcttccactgccacttttccccacgcgact |
| | | AGPNPSGNTTTSSNNLLFT | | ggcagcgactcatcaacaacaactgggg |
| | | SEEEIATTNPRDTDMFGQI | | actcaggccgaaatcgatgcgtgttaaaat |
| | | ADNNQNATTAPHIANLDA | | cttcaacatacaggtcaaggaggtcacga |
| | | MGIVPGMVWQNRDIYYQ | | cgtcaaacggcgagactacggtcgctaat |
| | | GPIWAKVPHTDGHFHPSP | | aaccttaccagcacggttcagatctttgcg |
| | | LMGGFGLKHPPPQIFIKNT | | gattcgacgtatgaactcccatacgtgatg |
| | | PVPANPNTTFSAARINSFL | | gacgccggtcaggaggggagctttcctc |
| | | TQYSTGQVAVQIDWEIQK | | cgtttcccaacgacgtctttatggttcccca |
| | | EHSKRWNPEVQFTSNYGT | | atacggatactgcggagttgtcactggaa |
| | | QNSMLWAPDNAGNYHEL | | aaaaccagaaccagacagacagaaatgc |
| | | RAIGSRFLTHHL | | ctatactgcctggaatactaccatcccaaa |
| | | | | tgctaagaactggcaacaattttgaagtca |
| | | | | gttaccaatagaaaaagttcctaccattca |
| | | | | atgtacgcgcacagccagagcctggaca |
| | | | | gaatgatgaatcctttactggatcagtacct |
| | | | | gtggcatctgcaatcgaccactaccggaa |
| | | | | attcccttaatcaaggaacagctaccacca |
| | | | | cgtacgggaaaattaccactggagacttt |
| | | | | gcctactacaggaaaaactggttgcctgg |
| | | | | agcctgcattaaacaacaaaaattacaaa |
| | | | | gaatgccaatcaaaactacaagattcccg |
| | | | | ccagcgggggagacgcccattaaagtat |
| | | | | gacacgcataccactctaaatgggcgatg |
| | | | | gagtaacatggctcctggacctccaatgg |
| | | | | caaccgcaggtgccggggactcggatttt |
| | | | | agcaacagccagctgatctttgccggacc |
| | | | | caatccgagcggtaacacgaccacatctt |
| | | | | caaacaatagttgatacctcagaagagg |
| | | | | agattgccacaacaaacccacgagacac |
| | | | | ggacatgtaggacagattgcagataataa |
| | | | | tcaaaatgccaccaccgcccctcacatcg |
| | | | | ctaacctggacgctatgggaattgttcccg |
| | | | | gaatggtctggcaaaacagagacatctac |
| | | | | taccagggccctatagggccaaggtccc |
| | | | | tcacacggacggacactttcacccttcgc |
| | | | | cgctgatgggaggatttggactgaaacac |
| | | | | ccgcctccacagattttcatcaaaaacacc |
| | | | | cccgtacccgccaatcccaatactaccttt |
| | | | | agcgctgcaaggattaattcttactgacgc |
| | | | | agtacagcaccggacaagttgccgttcag |
| | | | | atcgactgggaaattcagaaggagcattc |
| | | | | caaacgctggaatcccgaagttcaatttac |
| | | | | ttcaaactacggcactcaaaattctatgctg |
| | | | | tgggctcccgacaatgctggcaactacca |
| | | | | cgaactccgggctattgggtcccgtacct |
| | | | | cacccaccacttgtaa |

In some cases, an engineered AAV can include exogenous sequences from alternate serotypes. For example, a chimeric AAV, that can include sequences from at least two different AAV serotypes, can be generated. The term “serotype” can be a distinction with respect to an AAV having a capsid which is serologically distinct from other AAV serotypes. Serologic distinctiveness can be determined on the basis of the lack of cross-reactivity between antibodies to the AAV as compared to other AAVs. Cross-reactivity can be measured in a neutralizing antibody assay. For this assay polyclonal serum can be generated against a specific AAV in a rabbit or other suitable animal model using the adeno-associated viruses. In this assay, serum generated against a specific AAV can then be tested in its ability to neutralize either the same (homologous) or a heterologous AAV. The dilution that achieves 50% neutralization is considered the neutralizing antibody titer. If, for two AAVs, the quotient of the heterologous titer divided by the homologous titer is lower than 16 in a reciprocal manner, those two vectors are considered as the same serotype. Conversely, if the ratio of the heterologous titer over the homologous titer is 16 or more in a reciprocal manner, the two AAVs are considered distinct serotypes.

Homologous recombination can be used to generate capsids with new features and unique properties. Epitope coding sequences fused to either the N or C termini of the capsid coding sequences can be used to expose new peptides on the surface of the viral capsid without affecting gene function. In some embodiments, epitope sequences are inserted into specific positions in the capsid coding sequence by tagging the epitope into the coding sequences itself. In some embodiments, a chimeric capsid uses an epitope identified from a peptide library inserted into a specific position in the capsid coding sequence. The use of gene library to screen can be performed. For example, a screen of chimeras or mutant AAVs can be performed to identify chimeras and mutants that when used to transduce a cell confer increased transduction efficiency and/or increased expression of a transgene, such as an exogenous receptor.

Chimeric capsids in AAV vectors can expand the range of cell types that can be transfected and can increase the efficiency of transduction. Increased transduction or transfection can be from about a 10%, 20%, 30%, 40%, 50%, 60%, 70%, 80%, 90%, 100%, 150%, 200%, 250% increase to about a 300% increase as compared to a transduction using an AAV with an unmodified capsid. For example, increased transduction or transfection can be measured as compared to a WT AAV in terms of the detection of a transgene present (as a nucleic acid or polypeptide) on or in a cell. In some embodiments, an AAV comprising a chimeric capsid of two different AAV serotypes will have increased transduction efficiency as compared to one or both of the WT AAVs from which the capsid was derived. A chimeric capsid can contain a degenerate, recombined, shuffled, or otherwise modified Cap protein. For example, targeted insertion of receptor-specific ligands or single-chain antibodies at the N-terminus of VP proteins can be performed. An insertion of a lymphocyte antibody or target into an AAV can be performed to improve binding and infection of a T-cell. In some cases, virions having chimeric capsids (e.g., capsids containing a degenerate or otherwise modified Cap protein) can be made. To further alter the capsids of such virions, for example, to enhance or modify the binding affinity for a specific cell type, such as a lymphocyte, additional mutations can be introduced into the capsid of the virion. For example, suitable chimeric capsids can have ligand insertion mutations to facilitate viral targeting to specific cell types. The construction and characterization of AAV capsid mutants including insertion mutants, alanine screening mutants, and epitope tag mutants are described in Wu et al., J. Virol. 74:8635-45, 2000. Methods of making AAV capsid mutants are known, and include site-directed mutagenesis (Wu et al., J. Virol. 72:5919-5926); molecular breeding, nucleic acid, exon, and DNA family shuffling (Soong et al., Nat. Genet. 25:436-439, 2000; Coco et al., Nature Biotech. 2001; 19:354; and U.S. Pat. Nos. 5,837,458; 5,811,238; and 6,180,406; Kolkman and Stemmer, Nat. Biotech. 19:423-428, 2001; Fisch et al., Proceedings of the National Academy of Sciences 93:7761-7766, 1996; Christians et al., Nat. Biotech. 17:259-264, 1999); ligand insertions (Girod et al. Nat. Med. 9:1052-1056, 1999); cassette mutagenesis (Rueda et al. Virology 263:89-99, 1999; Boyer et al., J. Virol. 66:1031-1039, 1992); and the insertion of short random oligonucleotide sequences.

In some cases, a transcapsidation can be performed. Transcapsidation can be a process that involves the packaging of the ITR of one AAV serotype into the capsid of a different serotype. In another case, adsorption of receptor ligands to an AAV capsid surface can be performed and can be the addition of foreign peptides to the surface of an AAV capsid. In some cases, this can confer the ability to specifically target cells that no AAV serotype currently has a tropism towards, and this can greatly expand the uses of AAV as a gene therapy tool.

### AAP Modifications and Chimeras

In some embodiments, a modified AAV described herein comprises an AAP protein that comprises at least one amino acid modification compared to an AAP protein in a WT AAV of the same serotype. In some embodiments, said modified AAV comprises an AAP protein that comprises at least 1, 2, 3, 4, 5, 6, 7, 8, 9, or 10 amino acid modifications compared to WT AAP of the same serotype. Modifications can include amino acid substitutions, deletions, or additions. In some embodiments, said modified AAV comprises an AAP protein that comprises at least 1, 2, 3, 4, 5, 6, 7, 8, 9, or 10 amino acid substitutions compared to WT AAP of the same serotype. In some embodiments, said modified AAV comprises an AAP protein that comprises 1, 2, 3, 4, 5, 6, 7, 8, 9, or 10 amino acid substitutions compared to WT AAP of the same serotype.

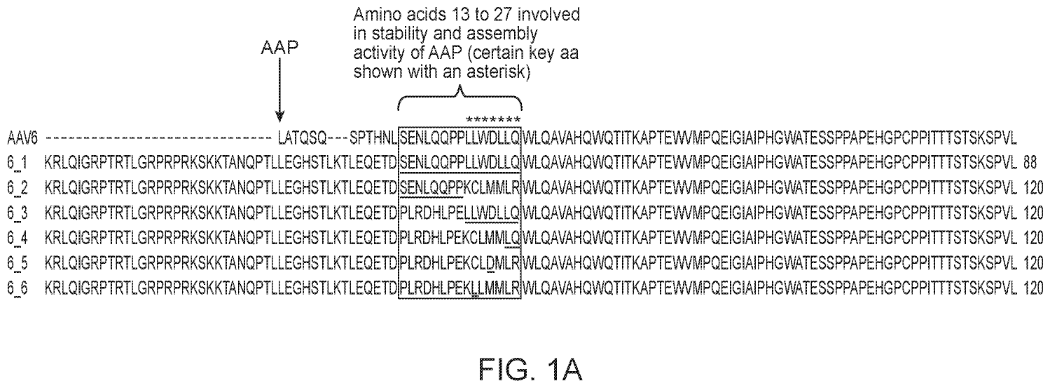

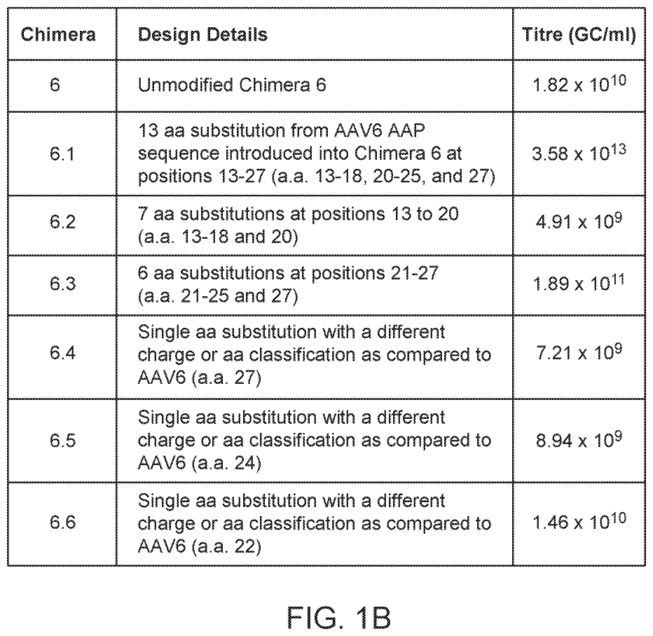

In some embodiments, said modified AAV comprises an AAP protein with a at least one amino acid modification (e.g., substitution) between amino acid positions 1 and 50, 5 and 40, 10 and 35, 13 and 27, 13 and 21, or 21 and 27 of the AAP protein, as compared to a WT AAP protein of the same serotype. In some embodiments, a mutation in AAP region is at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of said AAP protein, as compared to a WT AAP protein of the same serotype. One of ordinary skill in the art will readily understand that a sequence alignment of AAP sequences can be used to determine corresponding amino acid numbers in various AAP serotypes. An exemplary sequence alignment is provided in FIG. 1A. A variety of sequence alignment programs can be utilized for example, LALIGN, FFAS, BLAST, GeneWise, SIM, and SSEA.

Exemplary AAP chimeras are disclosed in Table 4 (nucleic acid sequences) and Table 5 (amino acid sequences). Exemplary WT AAP sequences are disclosed in Table 6.

In some embodiments, the chimera comprises an AAP protein encoded by a nucleic acid sequence in Table 4 or Table 5. In some embodiments, the chimera comprises an AAP protein comprising an amino acid sequence in Table 5. In some embodiments, the chimera comprises an AAP protein encoded by a nucleic acid sequence in Table 4. In some embodiments, the chimera comprises an AAP protein encoded by a nucleic acid sequence that shares at least 80%, 85%, 90%, 95%, 96%, 97%, 98%, 99% or 100% identity with SEQ ID NOs: 3-15. In some embodiments, the chimera comprises an AAP protein that comprises an amino acid sequence that shares at least 80%, 85%, 90%, 95%, 96%, 97%, 98%, 99% or 100% identity with SEQ ID NOs: 2, 16-25. In some embodiments, the chimera comprises an AAP protein encoded by a nucleic acid sequence that shares at least 99% or 100% identity with SEQ ID NOs: 3-15. In some embodiments, the chimera comprises an AAP protein that comprises an amino acid sequence that shares at least 99% or 100% identity with SEQ ID NOs: 2, 16-25.

### Transgenes, and Modified ITRs

In some embodiments, an AAV viral vector is used to introduce an exogenous transgene, such as a cellular receptor, into a cell. In some embodiments, said transgene encodes a functional protein. In some embodiments, said transgene encodes a cell surface receptor. In some embodiments said transgene encodes an intracellular protein. In some embodiments, said transgene encodes an exogenous T cell receptor (TCR), chimeric antigen receptor (CAR), or B cell receptor. In some embodiments, said transgene encodes an exogenous receptor that specifically binds to a cancer cells. In some embodiments, said transgene comprises homology arms for targeted integration of the transgene into the genome of a cell. In some embodiments, said transgene is randomly integrated into the genome of a cell.

In some embodiments, each end of the AAV single-stranded DNA genome contains an inverted terminal repeat (ITR). In some embodiments, said ITRs are the only cis-acting element required for genome replication and packaging. An ITR can be from any AAV serotype. For example, an ITR can be from the following AAV serotypes, AAV1, AAV2, AAV3, AAV4, AAV5, AAV6, AAV7, AAV8, AAV9, AAV10, AAV11, or AAV12. In some embodiments, said ITR is from AAV2.

### Helper Viruses

In some cases, the present disclosure provides construction of helper vectors that provide AAV Rep, Cap, and/or AAP proteins for producing stocks of virions composed of an AAV vector (e.g., a vector encoding an exogenous receptor sequence) and a chimeric capsid (e.g., a capsid containing a degenerate, recombined, shuffled or otherwise modified Cap protein). In some cases, a modification can involve the production of AAV cap nucleic acids that are modified, e.g., cap nucleic acids that contain portions of sequences derived from more than one AAV serotype (e.g., AAV serotypes 1-12). Such chimeric nucleic acids can be produced by a number of mutagenesis techniques. A method for generating chimeric cap genes can involve the use of degenerate oligonucleotides in an in vitro DNA amplification reaction. A protocol for incorporating degenerate mutations (e.g., polymorphisms from different AAV serotypes) into a nucleic acid sequence is described in Coco et al. (Nature Biotechnology 20:1246-1250, 2002). In this method, known as degenerate homoduplex recombination, “top-strand” oligonucleotides, that contain polymorphisms (degeneracies) from genes within a gene family, are constructed. Complementary degeneracies are engineered into multiple bridging “scaffold” oligonucleotides. A single sequence of annealing, gap-filling, and ligation steps results in the production of a library of nucleic acids capturing every possible permutation of the parental polymorphisms. Any portion of a capsid gene can be mutated using methods such as degenerate homoduplex recombination. Particular capsid gene sequences, however, are preferred. For example, critical residues responsible for binding of an AAV2 capsid to its cell surface receptor heparin sulfate proteoglycan (HSPG) have been mapped. Arginine residues at positions 585 and 588 appear to be critical for binding, as non-conservative mutations within these residues eliminate binding to heparin-agarose. Computer modeling of the AAV2 and AAV4 atomic structures identified seven hypervariable regions that overlap arginine residues 585 and 588, and that are exposed to the surface of the capsid. These hypervariable regions are thought to be exposed as surface loops on the capsid that mediates receptor binding. Therefore, these loops can be used as targets for mutagenesis in methods of producing chimeric virions with tropisms different from WT virions.

### Multiplicity of Infection

In some cases, a mutated or chimeric adeno-associated viral vector of the disclosure can be measured using multiplicity of infection (MOI). In some cases, MOI can refer to the ratio, or multiple of vector or viral genomes to the cells to which the nucleic can be delivered. In some cases, the MOI can be 1×10⁶ GC/mL. In some cases, the MOI can be 1×10⁵ GC/mL to 1×10⁷ GC/mL. In some cases, the MOI can be 1×10⁴ GC/mL to 1×10⁸ GC/mL. In some cases, recombinant viruses of the disclosure are at least about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI. In some cases, a mutated or chimeric adeno-associated viruses of this disclosure are from about 1×10⁸ GC/mL to about 3×10¹⁴ GC/mL MOI, or are at most about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI. In some cases, the viral vectors of the present disclosure are more effective and may have lower off-target effects during transduction of cells as compared to unmodified vectors. For example, a lower MOI of a modified virus may result in fewer off-target transgene insertions as compared to transducing a comparable cell with an unmodified vector.

### Methods of Producing Modified AAVs

The present disclosure provides methods and materials for producing recombinant modified AAV vectors and virions described herein. In some embodiments, the modified AAV vectors are chimeric and comprise a modified AAP protein. The present disclosure provides methods and materials for producing recombinant AAVs that can express one or more proteins of interest in a cell. As described herein, the methods and materials disclosed herein allow for high production or production of the proteins of interest at levels that achieve a therapeutic effect in vivo. An example of a protein of interest is an exogenous receptor. Exemplary exogenous receptors include, but are not limited to, a T-cell receptor (TCR), a B cell receptor, or a chimeric antigen receptor (CAR).

To generate AAV virions or viral particles, an AAV expression vector is introduced into a suitable host cell using known techniques, such as by transfection. Transfection techniques are known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Suitable transfection methods include, but are not limited to, calcium phosphate co-precipitation, direct micro-injection, electroporation, liposome mediated gene transfer, and nucleic acid delivery using high-velocity microprojectiles, which are known in the art.

In some embodiments, methods for producing a recombinant AAV virions include providing a packaging cell line with a viral construct comprising a 5′ inverted terminal repeat (ITR) of AAV and a 3′ AAV ITR (such as those described herein), helper functions for generating a productive AAV infection, and AAV cap genes; and recovering a recombinant AAV virions from the supernatant of the packaging cell line. Various types of cells can be used as the packaging cell line. For example, packaging cell lines include, but are not limited to, HEK 293 cells, HeLa cells, and Vero cells. In some embodiments, supernatant of the packaging cell line is treated by PEG precipitation for concentrating the virus. In some embodiments, a centrifugation step is be used to concentrate a virus. For example a column can be used to precipitate virus during a centrifugation. In some embodiments, a precipitation occurs at no more than about 4° C. (for example about 3° C., about 2° C., about 1° C., or about 1° C.) for at least about 2 hours, at least about 3 hours, at least about 4 hours, at least about 6 hours, at least about 9 hours, at least about 12 hours, or at least about 24 hours. In some embodiments, the recombinant AAV is isolated from the PEG-precipitated supernatant by low-speed centrifugation followed by cesium chloride gradient. In some embodiment, the low-speed centrifugation is carried out at about 4000 rpm, about 4500 rpm, about 5000 rpm, or about 6000 rpm for about 20 minutes, about 30 minutes, about 40 minutes, about 50 minutes or about 60 minutes. In some embodiments, recombinant AAV is isolated from PEG-precipitated supernatant by centrifugation at about 5000 rpm for about 30 minutes followed by purification using a cesium chloride gradient. In some embodiments, cesium chloride purification can be replaced with IDX gradient ultracentrifugation. Supernatant can be collected at about 12 hours, about 24 hours, about 36 hours, about 48 hours, about 72 hours, about 96 hours, or about 120 hours after transfection, or a time between any of these two time points after a transfection. Supernatant can also be purified, concentrated, or a combination thereof. For example, a concentration or viral titer can be determined by qPCR or silver stain. An optimal viral titer can vary depending on cell type to be transduced. A range of virus can be from about 1000 MOI to 2000 MOI, from 1500 MOI to 2500 MOI, from 2000 MOI to 3000 MOI, from 3000 MOI to 4000 MOI, from 4000 MOI to 5000 MOI, from 5000 MOI to 6000 MOI, from 6000 MOI to 7000 MOI, from 7000 MOI to 8000 MOI, from 8000 MOI to 9000 MOI, or from 9000 MOI to 10,000 MOI. The For example, to infect 1 million cells using a MOI of 10,000, one will need 10,000×1,000,000=10¹⁰ GC.

Introduction of plasmids or viruses into a host cell can also be accomplished using techniques known to those of ordinary skill in the art and as discussed throughout the specification. In some cases, standard transfection techniques are used, e.g., calcium phosphate transfection or electroporation, and/or infection by hybrid adenovirus/AAV vectors into cell lines such as HEK 293 (a human embryonic kidney cell line containing functional adenovirus E1 genes which provides trans-acting E1 proteins). One of skill in the art will readily understand that the novel AAV sequences described herein can be readily adapted for use in these and other viral vector systems for in vitro, ex vivo, or in vivo gene delivery. Similarly, one of skill in the art can readily select other fragments of the AAV genome for use in a variety of AAV and non-AAV vector systems. Such vectors systems can include, e.g., lentiviruses, retroviruses, poxviruses, vaccinia viruses, and adenoviral systems, among others. Selection of these vector systems is not a limitation of the present disclosure.

In some embodiments, helper functions are provided by one or more helper plasmids or helper viruses comprising adenoviral helper genes. Non-limiting examples of the adenoviral helper genes include E1A, E1B, E2A, E4 and VA, which can provide helper functions to AAV packaging. In some cases, an AAV cap gene can be present in a plasmid. A plasmid can further comprise an AAV rep gene. In other cases, an AAP gene can be present in a plasmid.

Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US Publication No. 20110201088, helper vectors pHELP (Applied Viromics). A skilled artisan will appreciate that any helper virus or helper plasmid of AAV that can provide adequate helper function to AAV can be used herein. The recombinant AAV viruses disclosed herein can also be produced using any convention methods known in the art suitable for producing infectious recombinant AAV. In some cases, a recombinant AAV can be produced by using a cell line that stably expresses some of the necessary components for AAV particle production. For example, a plasmid (or multiple plasmids) comprising AAV rep and cap genes, and a selectable marker, such as a neomycin resistance gene, can be integrated into the genome of a cell (the packaging cells). The packaging cell line can then be co-infected with a helper virus (e.g., adenovirus providing the helper functions) and the viral vector comprising the 5′ and 3′ AAV ITR and the nucleotide sequence encoding the protein(s) of interest. In another non-limiting example, adenovirus or baculovirus rather than plasmids can be used to introduce rep and cap genes into packaging cells. As yet another non-limiting example, both the viral vector containing the 5′ and 3′ AAV ITRs and the rep and cap genes can be stably integrated into the DNA of producer cells, and the helper functions can be provided by a WT adenovirus to produce the recombinant AAV.

In some cases, a packaging plasmid can contain all the necessary viral proteins on one plasmid to enable packing of an ITR-flanked donor template into replication-incompetent virus particles.

Suitable host cells that can be used to produce AAV virions or viral particles include yeast cells, insect cells, microorganisms, and mammalian cells. Various stable human cell lines can be used, including, but not limited to, HEK 293 cells. Host cells can be engineered to provide helper functions in order to replicate and encapsidate nucleotide sequences flanked by AAV ITRs to produce viral particles or AAV virions. AAV helper functions can be provided by AAV-derived coding sequences that are expressed in host cells to provide AAV gene products in trans for AAV replication and packaging. AAV virus can be made replication-competent or replication-incompetent. In general, a replication-incompetent AAV virus lacks one or more AAV packaging genes. Cells can be contacted with viral vectors, viral particles, or virus as described herein in vitro, in vivo, or ex vivo. In some embodiments, cells that are contacted in vitro can be derived from established cell lines or primary cells derived from a subject, either modified ex vivo for return to the subject, or allowed to grow in culture in vitro. In some aspects, a virus is used to deliver a viral vector into primary cells ex vivo to modify the cells, such as introducing an exogenous nucleic acid sequence, a transgene, or an engineered cell receptor in an immune cell, or a T-cell in particular, followed by expansion, selection, or limited number of passages in culture before such modified cells are returned back to the subject. In some aspects, such modified cells are used in cell-based therapy to treat a disease or condition, including cancer.

Any conventional methods suitable for purifying AAV can be used in the embodiments described herein to purify the recombinant AAV. For example, the recombinant AAV can be isolated and purified from packaging cells and/or the supernatant of the packaging cells. In some embodiments, the AAV can be purified by separation method using a cesium chloride gradient. Also, US Patent Publication No. 20020136710 describes another non-limiting example of method for purifying AAV, in which AAV was isolated and purified from a sample using a solid support that includes a matrix to which an artificial receptor or receptor-like molecule that mediates AAV attachment is immobilized.

Disclosed herein can be a functional AAV. A functional AAV can be an AAV characterized by the ability to produce viral particles with equivalent or greater packaging and transduction efficiency as any one of a WT AAV, such as AAV6. Function can be assessed in a pseudotyping setting with AAV6 rep and AAV6 ITRs. Thus, an altered parental AAV can be constructed using conventional techniques and the AAV vector can be considered functional if virus is produced from the parental AAV at titers of at least 50% when compared to production of a WT AAV such as AAV6. Further, the ability of AAV to transduce cells can be readily determined by one of skill in the art. For example, a parental AAV can be constructed such that it contains a marker gene which allows easy detection of virus. For example, an AAV can contain eGFP or another transgene which allows fluorescent detection. Where the AAV contains CMV-eGFP, when the virus produced from the altered parental AAV capsid is transduced into HEK 293 cells at a multiplicity of infection of 10⁴, function is demonstrated where transduction efficiency is greater than 5% GFP fluorescence of total cells in a context where the cells were pretreated with WT human adenovirus type 5 at a multiplicity of infection of 20 for 2 hours.

### Methods of Engineering Cells Using Modified AAVs and Populations of Engineered Cells

Provided herein are compositions of cells engineered using a modified AAV described herein. In some embodiments, said cells are immune cells. In some embodiments, said cells are primary cells. In some embodiments, said cells are engineered ex vivo. In some embodiments, said cells are primary cells. In some embodiments, said cells are engineered ex vivo and administered to the subject the cells were obtained from. In some embodiments, said cells are primary cells. In some embodiments, said cells are engineered ex vivo and administered to a subject different from the subject (but of the same species) than the cells were obtained from. In some embodiments, the cells comprise T cells (e.g., CD4+ T cells, CD8+ T cells), tumor infiltrating lymphocytes (TILs), B cells, NK cells, NK T cells, macrophages, monocytes, or dendritic cells.

In some embodiments, said cells comprise a transgene integrated into the genome of the cell, wherein said integration is mediated by a modified AAV described herein. In some embodiments, the transgene encodes a cell surface receptor. In some embodiments, the transgene encodes a T cell receptor (TCR), B cell receptor, or chimeric antigen receptor (CAR). In some embodiments, the transgene is integrated into a safe harbor locus, e.g., HPRT, AAVS1, CCR5, or Rosa26. In some embodiments, the transgene is a TCR or a CAR and is integrated into TRAC or TCRB locus. In some embodiments, said transgene is integrated into a gene encoding an immune checkpoint protein. In some embodiments, said immune checkpoint protein is selected from the group consisting of cytokine inducible SH2-containing protein (CISH), programmed cell death 1 (PD-1), cytotoxic T-lymphocyte-associated protein 4 (CTLA4), adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C-C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), T-cell receptor alpha locus (TRA), T cell receptor beta locus (TRB), egl-9 family hypoxia-inducible factor 1 (EGLN1), egl-9 family hypoxia-inducible factor 2 (EGLN2), egl-9 family hypoxia-inducible factor 3 (EGLN3), and protein phosphatase 1 regulatory subunit 12C (PPP1R12C).

In some embodiments, said cells comprise an alteration (e.g., disruption) of at least one gene in the genome, wherein said alteration (e.g., disruption) results in inhibition or decrease in expression of a function protein encoded by said gene. In some embodiments, said disruption is mediated by integration of a transgene into the genome of the cell, wherein said integration is mediated by a modified AAV described herein. In some embodiments, said disruption is mediated by a CRISPR system, TALEN system, Zinc Finger nuclease system, transposon-based system, ZEN system, meganuclease system, or Mega-TAL system. In some embodiments, said disruption is mediated by a CRISPR system that comprises a gRNA that binds to a target DNA sequence and a Cas endonuclease. In some embodiments, said Cas endonuclease is Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof or modified versions thereof. In some embodiments, said Cas endonuclease is Cas9. In some embodiments, the gRNA and cas9 endonuclease are transfected into said cells (e.g., via electroporation). In some embodiments, said disruption is in a gene (coding sequence) or regulatory element of a gene (e.g., promoter or enhancer) of a gene encoding an immune checkpoint protein. In some embodiments, said disruption is in a gene (coding sequence) or regulatory element of a gene (e.g., promoter or enhancer) of a gene selected from the group consisting of cytokine inducible SH2-containing protein (CISH), programmed cell death 1 (PD-1), cytotoxic T-lymphocyte-associated protein 4 (CTLA4), adenosine A2a receptor (ADORA), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1 (AAVS1), or chemokine (C-C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, or guanylate cyclase 1, soluble, beta 3 (GUCY1B3), T-cell receptor alpha locus (TRA), T cell receptor beta locus (TRB), egl-9 family hypoxia-inducible factor 1 (EGLN1), egl-9 family hypoxia-inducible factor 2 (EGLN2), egl-9 family hypoxia-inducible factor 3 (EGLN3), and protein phosphatase 1 regulatory subunit 12C (PPP1R12C).

### Methods of Identifying AAV Serotypes

Disclosed herein are, inter alia, methods of identifying an AAV serotype. In some embodiments, an AAV serotype is identified using a PCR approach. Using PCR, one or ordinary skill in the art can amplify regions of the AAV genome, principally a 255 bp fragment of the capsid gene called the “signature region” in which the 5′ and 3′ sequences are conserved but the central sequence can be variable and unique to each AAV serotype. In some embodiments, the signature region is from about 50 bp, 75 bp, 80 bp, 100 bp, 125 bp, 150 bp, 175 bp, 200 bp, 225 bp, 255 bp, 260 bp, 270 bp, 280 bp, 290 bp, 300 bp, 325 bp, 350 bp, 375 bp, 400 bp, or up to about 450 bp. Primers can be designed to anneal to conserved regions of the rep and cap genes to amplify and identify novel AAV serotypes (e.g., as shown in Gao et al., 2002). The signature region of AAV can be amplified from genomic DNA (gDNA). gDNA can be extracted from a mammalian cell or a non-mammalian cell. In some cases, gDNA can be extracted from a cell line such as HCT116, HEK293, Jurkat, U-937, NCI-H838, pDG, AAV DJ, or a combination thereof. In some cases, gDNA can be extracted from a human cell. gDNA can be extracted from peripheral blood mononuclear cells (PBMCs). gDNA can be extracted from liver, heart, brain, kidney, lung, spleen, bone, skin, buccal, blood, saliva, and the like.

### Methods of Using Modified AAVs and Cells Produced Using Modified AAVs to Treat Cancer

The present disclosure provides, inter alia, methods of using modified AAVs described herein to treat cancer. In some embodiments, cells engineered ex vivo using a modified AAV described herein are administered to a subject in need thereof, (e.g., a subject having cancer). In some embodiments, said cells are administered to an autologous subject. In some embodiments, said cells are administered to an allogenic subject. The dosing and regimen of administration can be determined by a person of ordinary skill in the art. In some embodiments, 0.1 to 10.0×10⁶ cells per kg body weight of said subject, 0.1 to 9.0×10⁶ cells per kg body weight of said subject, 0.1 to 8.0×10⁶ cells per kg body weight of said subject, 0.1 to 7.0×10⁶ cells per kg body weight of said subject, 0.1 to 6.0×10⁶ cells per kg body weight of said subject, 0.1 to 5.0×10⁶ cells per kg body weight of said subject, 0.1 to 4.0×10⁶ cells per kg body weight of said subject, 0.1 to 3.0×10⁶ cells per kg body weight of said subject, 0.1 to 2.0×10⁶ cells per kg body weight of said subject, or 0.1 to 1.0×10⁶ cells per kg body weight of said subject are administered to said subject. In some embodiments, 0.1 to 10×10⁸ cells, 0.1 to 9×10⁸ cells, 0.1 to 8×10⁸ cells, 0.1 to 7×10⁸ cells, 0.1 to 6×10⁸ cells, 0.1 to 5×10⁸ cells, 0.1 to 4×10⁸ cells, 0.1 to 3×10⁸ cells, 0.1 to 2×10⁸ cells, or 0.1 to 1×10⁸ cells are administered to said subject. In some embodiments, said cells are immune cells (e.g., immune cells described herein). In some embodiments, said immune cells are T cells, tumor infiltrating lymphocytes, B cells, NK cells, macrophages, monocytes, or dendritic cells.

In some embodiments, the cancer is a solid tumor. In some embodiments, the cancer is a hematological malignancy. In some embodiments, the cancer is acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. In some embodiments, the cancer is metastatic.

### EXAMPLES

The present disclosure will be described in greater detail by way of the following specific examples. The following examples are offered for illustrative purposes, and are not intended to limit the disclosure in any manner. Those of skill in the art will readily recognize a variety of non-critical parameters that can be changed or modified to yield alternative embodiments according to the invention. All patents, patent applications, and printed publications listed herein are incorporated herein by reference in their entirety.

### Example 1—AAP Nucleotide and Polypeptide Sequences

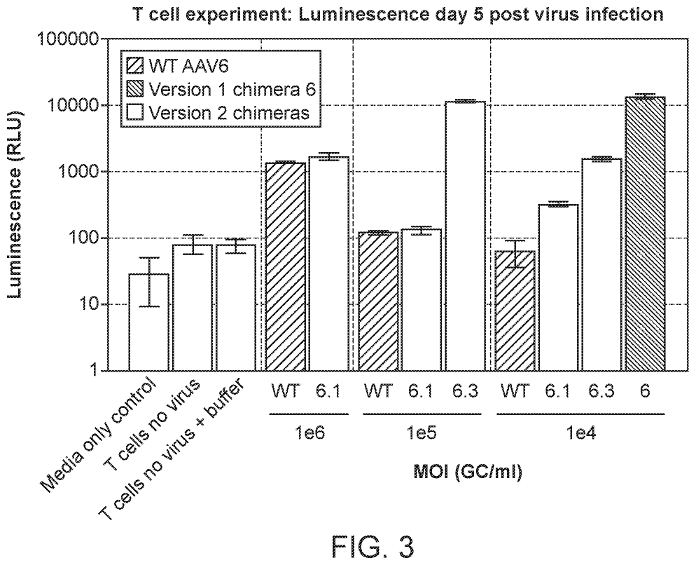

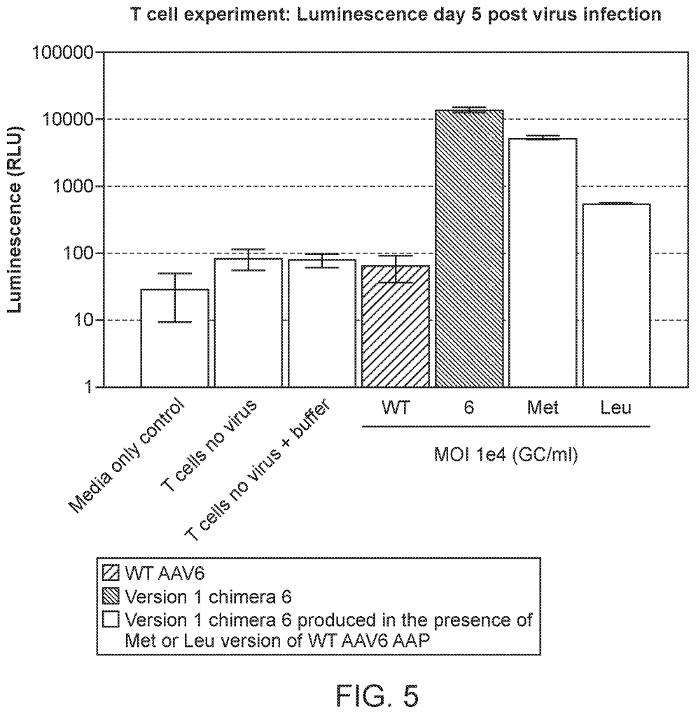

A number of AAV chimeras (e.g., having VP1, VP2, and VP3 sequences from at least two different AAV serotypes, resulting in chimeric AAP sequences) were identified and isolated. Among the chimeras, chimera 6, which has VP1 and VP2 sequences from AAV serotype 12 and VP3 sequence from AAV serotype 6 with a chimeric AAP sequence of AAV serotype 12 and 6, significantly increased AAV infectivity (see FIG. 3 and FIG. 5). To further improve the quality of chimera 6 (e.g., virus titer), point mutations were made in a region that is important for the stability and assembly activity of AAP proteins—amino acids 13-27 (the amino acid numbers are with respect to WT AAV6 AAP sequences; FIG. 1A). For example, chimera 6.1 has 13 amino acid substitutions (amino acids 13-18, amino acids 20-25, and amino acid 27) that restore the amino acid sequence of chimera 6.1 to that of WT AAV6 in this region (amino acids 13-27). Likewise, chimera 6.2 has seven amino acid substitutions (amino acids 13-18 and amino acid 20) and chimera 6.3 has six amino acid substitutions (amino acids 21-25 and amino acid 27) that restore the amino acid sequence of chimeras 6.2 and 6.3 in this region to that of WT AAV6 (amino acids 13-20 and amino acids 21-27, respectively). Chimeras 6.4, 6.5, and 6.6 have one amino acid substitution at amino acid 27, 24, and 22, respectively. Table 4 below describes the nucleic acid sequences of AAP chimeras; and Table 5 provides the corresponding amino acid sequences of the AAP chimeras.

TABLE 3
AAP Nucleic Acid and amino acid sequence of WT AAV6.

| | AAP nucleic acid sequence | | AAP amino acid sequence |
|---|---|---|---|
| | (portion corresponding to | | (portion corresponding |
| SEQ | amino acids 13-27 of AAV6 | SEQ ID | to amino acids 13-27 of |
| ID NO: | bold and underlined) | NO: | AAV6 bold and underlined) |
| 1 | ctggcgactcagagtcagtccccgacccaca | 2 | LATQSQSPTHNLSENLQQPPLLW |
| | acctctcggagaacctccagcaacccccgc | | DLLQWLQAVAHQWQTITKAPTE |
| | tgctgtgggacctactacaatggcttcaggc | | WVMPQEIGIAIPHGWATESSPPAP |
| | ggtggcgcaccaatggcagacaataacgaa | | EHGPCPPITTTSTSKSPVLQRGPAT |
| | ggcgccgacggagtgggtaatgcctcagga | | TTTTSATAPPGGILISTDSTAISHH |
| | aattggcattgcgattccacatggctgggcga | | VTGSDSSTTIGDSGPRDSTSSSSTS |
| | cagagtcatcaccaccagcacccgaacatgg | | KSRRSRRMMASRPSLITLPARFKS |
| | gccttgcccacctataacaaccacctctacaa | | SRTRSTSCRTSSALRTRAASLRSR |
| | gcaaatctccagtgcttcaacgggggccagc | | RTCS |
| | aacgacaaccactacttcggctacagcaccc | | |
| | cctgggggtattttgatttcaacagattccactg | | |
| | ccatttctcaccacgtgactggcagcgactcat | | |
| | caacaacaattggggattccggcccaagaga | | |
| | ctcaacttcaagctcttcaacatccaagtcaag | | |
| | gaggtcacgacgaatgatggcgtcacgacca | | |
| | tcgctaataaccttaccagcacggttcaagtctt | | |
| | ctcggactcggagtaccagttgccgtacgtcc | | |
| | tcggctctgcgcaccagggctgcctccctccg | | |
| | ttcccggcggacgtgttcatga | | |

TABLE 4
AAP Nucleic Acid Sequences of Chimeras (Chimeras 3, 4, 5, and 6 have AAP
sequences formed from two different AAV serotypes.)

| | SEQ ID | |
|---|---|---|
| Chimera | NO: | AAP nucleic acid sequence |
| Chimera 2 | 3 | ctggcgactcagagtcagtccccgacccacaacctctcggagaacctccagcaacccccgctgctgtg |
| AAV5VP1u- | | ggacctactacaatggcttcaggcggtggcgcaccaatggcagacaataacgaaggcgccgacggag |
| AAV6VP2/3 | | tgggtaatgcctcaggaaattggcattgcgattccacatggctgggcgacagagtcatcaccaccagca |
| | | cccgaacatgggccttgcccacctataacaaccacctctacaagcaaatctccagtgcttcaacggggg |
| | | ccagcaacgacaaccactacttcggctacagcaccccctgggggtattttgatttcaacagattccactgc |
| | | catttctcaccacgtgactggcagcgactcatcaacaacaattggggattccggcccaagagactcaact |
| | | tcaagctcttcaacatccaagtcaaggaggtcacgacgaatgatggcgtcacgaccatcgctaataacct |
| | | taccagcacggttcaagtcttctcggactcggagtaccagttgccgtacgtcctcggctctgcgcaccag |
| | | ggctgcctccctccgttcccggcggacgtgttcatga |
| Chimera 3 | 4 | ttgaatccccccagcagcccgactcctccacgggtatcggcaaaaaaggcaagcagccggctaaaaa |
| rAAV4P1/2- | | gaagctcgttttcgaagacgaaactggagcaggcgacggaccccctgagggatcaacttccggagcc |
| AAV6VP3 | | atgtctgatgacagtgagatggcttcaggcggtggcgcaccaatggcagacaataacgaaggcgccga |
| | | cggagtgggtaatgcctcaggaaattggcattgcgattccacatggctgggcgacagagtcatcaccac |
| | | cagcacccgaacatgggccttgcccacctataacaaccacctctacaagcaaatctccagtgcttcaacg |
| | | ggggccagcaacgacaaccactacttcggctacagcaccccctgggggtattttgatttcaacagattcc |
| | | actgccatttctcaccacgtgactggcagcgactcatcaacaacaattggggattccggcccaagagact |
| | | caacttcaagctcttcaacatccaagtcaaggaggtcacgacgaatgatggcgtcacgaccatcgctaat |
| | | aaccttaccagcacggttcaagtcttctcggactcggagtaccagttgccgtacgtcctcggctctgcgca |
| | | ccagggctgcctccctccgttcccggcggacgtgttcatga |
| Chimera 4 | 5 | acgaccactttccaaaaagaaagaaggctcggaccgaagaggactccaagccttccacctcgtcagac |
| rAAV5VP1/2- | | gccgaagctggacccagcggatcccagcagctgcaaatcccagcccaaccagcctcaagtttgggag |
| AAV6VP3 | | ctgatacaatggcttcaggcggtggcgcaccaatggcagacaataacgaaggcgccgacggagtggg |
| | | taatgcctcaggaaattggcattgcgattccacatggctgggcgacagagtcatcaccaccagcacccg |
| | | aacatgggccttgcccacctataacaaccacctctacaagcaaatctccagtgcttcaacgggggccag |
| | | caacgacaaccactacttcggctacagcaccccctgggggtallllgatttcaacagattccactgccattt |
| | | ctcaccacgtgactggcagcgactcatcaacaacaattggggattccggcccaagagactcaacttcaa |
| | | gctcttcaacatccaagtcaaggaggtcacgacgaatgatggcgtcacgaccatcgctaataaccttacc |
| | | agcacggttcaagtcttctcggactcggagtaccagttgccgtacgtcctcggctctgcgcaccagggct |
| | | gcctccctccgttcccggcggacgtgttcatga |
| Chimera 5 | 6 | agtcaccacaagagcccgactcctcctcgggcatcggcaaaaaaggcaaacaaccagccagaaaga |
| rAAV11VP1/2- | | ggctcaactttgaagaggacactggagccggagacggaccccctgaaggatcagataccagcgccat |
| AAV6VP3 | | gtcttcagacattgaaatggcttcaggcggtggcgcaccaatggcagacaataacgaaggcgccgacg |
| | | gagtgggtaatgcctcaggaaattggcattgcgattccacatggctgggcgacagagtcatcaccacca |
| | | gcacccgaacatgggccttgcccacctataacaaccacctctacaagcaaatctccagtgcttcaacgg |
| | | gggccagcaacgacaaccactacttcggctacagcaccccctgggggtattttgatttcaacagattcca |
| | | ctgccatttctcaccacgtgactggcagcgactcatcaacaacaattggggattccggcccaagagactc |
| | | aacttcaagctcttcaacatccaagtcaaggaggtcacgacgaatgatggcgtcacgaccatcgctaata |
| | | accttaccagcacggttcaagtcttctcggactcggagtaccagttgccgtacgtcctcggctctgcgcac |
| | | cagggctgcctccctccgttcccggcggacgtgttcatga |
| Chimera 6 | 7 | aaaagactccaaatcggccgaccaacccggactctgggaaggccccggccaagaaaaagcaaaaag |
| AAV12VP1/2- | | acggcgaaccagccgactctgctagaaggacactcgactttgaagactctggagcaggagacggacc |
| AAV6VP3 | | ccctgagggatcatcttccggagaaatgtctcatgatgctgagatggcttcaggcggtggcgcaccaatg |
| | | gcagacaataacgaaggcgccgacggagtgggtaatgcctcaggaaattggcattgcgattccacatg |
| | | gctgggcgacagagtcatcaccaccagcacccgaacatgggccttgcccacctataacaaccacctct |
| | | acaagcaaatctccagtgcttcaacgggggccagcaacgacaaccactacttcggctacagcaccccc |
| | | tgggggtattttgatttcaacagattccactgccatttctcaccacgtgactggcagcgactcatcaacaac |
| | | aattggggattccggcccaagagactcaacttcaagctcttcaacatccaagtcaaggaggtcacgacg |
| | | aatgatggcgtcacgaccatcgctaataaccttaccagcacggttcaagtcttctcggactcggagtacc |
| | | agttgccgtacgtcctcggctctgcgcaccagggctgcctccctccgttcccggcggacgtgttcatga |
| Chimera 7 | 8 | ctggcgactcagagtcagtccccgacccacaacctctcggagaacctccagcaacccccgctgctgtg |
| AAV4VP1u- | | ggacctactacaatggcttcaggcggtggcgcaccaatggcagacaataacgaaggcgccgacggag |
| AAV6VP2/3 | | tgggtaatgcctcaggaaattggcattgcgattccacatggctgggcgacagagtcatcaccaccagca |
| | | cccgaacatgggccttgcccacctataacaaccacctctacaagcaaatctccagtgcttcaacggggg |
| | | ccagcaacgacaaccactacttcggctacagcaccccctgggggtattttgatttcaacagattccactgc |
| | | catttctcaccacgtgactggcagcgactcatcaacaacaattggggattccggcccaagagactcaact |
| | | tcaagctcttcaacatccaagtcaaggaggtcacgacgaatgatggcgtcacgaccatcgctaataacct |
| | | taccagcacggttcaagtcttctcggactcggagtaccagttgccgtacgtcctcggctctgcgcaccag |
| | | ggctgcctccctccgttcccggcggacgtgttcatga |
| Chimera 8 | 9 | ctggcgactcagagtcagtccccgacccacaacctctcggagaacctccagcaacccccgctgctgtg |
| AAV12VP1u- | | ggacctactacaatggcttcaggcggtggcgcaccaatggcagacaataacgaaggcgccgacggag |
| AAV6VP2/3 | | tgggtaatgcctcaggaaattggcattgcgattccacatggctgggcgacagagtcatcaccaccagca |
| | | cccgaacatgggccttgcccacctataacaaccacctctacaagcaaatctccagtgcttcaacggggg |
| | | ccagcaacgacaaccactacttcggctacagcaccccctgggggtattttgatttcaacagattccactgc |
| | | catttctcaccacgtgactggcagcgactcatcaacaacaattggggattccggcccaagagactcaact |
| | | tcaagctcttcaacatccaagtcaaggaggtcacgacgaatgatggcgtcacgaccatcgctaataacct |
| | | taccagcacggttcaagtcttctcggactcggagtaccagttgccgtacgtcctcggctctgcgcaccag |
| | | ggctgcctccctccgttcccggcggacgtgttcatga |
| Chimera | 10 | aaaagactccaaatcggccgaccaacccggactctgggaaggccccggccaagaaaaagcaaaaag |
| 6.1 | | acggcgaaccagccgactctgctagaaggacactcgactttgaagactctggagcaggagacggact |
| AAV12VP1u- | | cggagaacctccagcaacccccgctgctgtgggacctactacaatggcttcaggcggtggcgcacc |
| AAV6VP2/3 | | aatggcagacaataacgaaggcgccgacggagtgggtaatgcctcaggaaattggcattgcgattcca |
| | | catggctgggcgacagagtcatcaccaccagcacccgaacatgggccttgcccacctataacaaccac |
| | | ctctacaagcaaatctccagtgcttcaacgggggccagcaacgacaaccactacttcggctacagcacc |
| | | ccctgggggtattttgatttcaacagattccactgccatttctcaccacgtgactggcagcgactcatcaac |
| | | aacaattggggattccggcccaagagactcaacttcaagctcttcaacatccaagtcaaggaggtcacg |
| | | acgaatgatggcgtcacgaccatcgctaataaccttaccagcacggttcaagtcttctcggactcggagt |
| | | accagttgccgtacgtcctcggctctgcgcaccagggctgcctccctccgttcccggcggacgtgttcat |
| | | ga |
| Chimera | 11 | aaaagactccaaatcggccgaccaacccggactctgggaaggccccggccaagaaaaagcaaaaag |
| 6.2 | | acggcgaaccagccgactctgctagaaggacactcgactttgaagactctggagcaggagacggact |
| AAV12VP1u- | | cggagaacctccagcaacccccgaaatgtctcatgatgctgagatggcttcaggcggtggcgcacc |
| AAV6VP2/3 | | aatggcagacaataacgaaggcgccgacggagtgggtaatgcctcaggaaattggcattgcgattcca |
| | | catggctgggcgacagagtcatcaccaccagcacccgaacatgggccttgcccacctataacaaccac |
| | | ctctacaagcaaatctccagtgcttcaacgggggccagcaacgacaaccactacttcggctacagcacc |
| | | ccctgggggtattttgatttcaacagattccactgccatttctcaccacgtgactggcagcgactcatcaac |
| | | aacaattggggattccggcccaagagactcaacttcaagctcttcaacatccaagtcaaggaggtcacg |
| | | acgaatgatggcgtcacgaccatcgctaataaccttaccagcacggttcaagtcttctcggactcggagt |
| | | accagttgccgtacgtcctcggctctgcgcaccagggctgcctccctccgttcccggcggacgtgttcat |
| | | ga |
| Chimera | 12 | aaaagactccaaatcggccgaccaacccggactctgggaaggccccggccaagaaaaagcaaaaag |
| 6.3 | | acggcgaaccagccgactctgctagaaggacactcgactttgaagactctggagcaggagacggacc |
| AAV12VP1u- | | ccctgagggatcatcttccggagctgctgtgggacctactacaatggcttcaggcggtggcgcacca |
| AAV6VP2/3 | | atggcagacaataacgaaggcgccgacggagtgggtaatgcctcaggaaattggcattgcgattccac |
| | | atggctgggcgacagagtcatcaccaccagcacccgaacatgggccttgcccacctataacaaccacc |
| | | tctacaagcaaatctccagtgcttcaacgggggccagcaacgacaaccactacttcggctacagcaccc |
| | | cctgggggtattttgatttcaacagattccactgccatttctcaccacgtgactggcagcgactcatcaaca |
| | | acaattggggattccggcccaagagactcaacttcaagctcttcaacatccaagtcaaggaggtcacga |
| | | cgaatgatggcgtcacgaccatcgctaataaccttaccagcacggttcaagtcttctcggactcggagta |
| | | ccagttgccgtacgtcctcggctctgcgcaccagggctgcctccctccgttcccggcggacgtgttcatg |
| | | a |
| Chimera | 13 | aaaagactccaaatcggccgaccaacccggactctgggaaggccccggccaagaaaaagcaaaaag |
| 6.4 | | acggcgaaccagccgactctgctagaaggacactcgactttgaagactctggagcaggagacggacc |
| AAV12VPlu- | | ccctgagggatcatcttccggagaaatgtctcatgatgctgcaatggcttcaggcggtggcgcacca |
| AAV6VP2/3 | | atggcagacaataacgaaggcgccgacggagtgggtaatgcctcaggaaattggcattgcgattccac |
| | | atggctgggcgacagagtcatcaccaccagcacccgaacatgggccttgcccacctataacaaccacc |
| | | tctacaagcaaatctccagtgcttcaacgggggccagcaacgacaaccactacttcggctacagcaccc |
| | | cctgggggtattttgatttcaacagattccactgccatttctcaccacgtgactggcagcgactcatcaaca |
| | | acaattggggattccggcccaagagactcaacttcaagctcttcaacatccaagtcaaggaggtcacga |
| | | cgaatgatggcgtcacgaccatcgctaataaccttaccagcacggttcaagtcttctcggactcggagta |
| | | ccagttgccgtacgtcctcggctctgcgcaccagggctgcctccctccgttcccggcggacgtgttcatg |
| | | a |
| Chimera | 14 | aaaagactccaaatcggccgaccaacccggactctgggaaggccccggccaagaaaaagcaaaaag |
| 6.5 | | acggcgaaccagccgactctgctagaaggacactcgactttgaagactctggagcaggagacggacc |
| AAV12VP1u- | | ccctgagggatcatcttccggagaaatgtctcgacatgctgagatggcttcaggcggtggcgcacc |
| AAV6VP2/3 | | aatggcagacaataacgaaggcgccgacggagtgggtaatgcctcaggaaattggcattgcgattcca |
| | | catggctgggcgacagagtcatcaccaccagcacccgaacatgggccttgcccacctataacaaccac |
| | | ctctacaagcaaatctccagtgcttcaacgggggccagcaacgacaaccactacttcggctacagcacc |
| | | ccctgggggtattttgatttcaacagattccactgccatttctcaccacgtgactggcagcgactcatcaac |
| | | aacaattggggattccggcccaagagactcaacttcaagctcttcaacatccaagtcaaggaggtcacg |
| | | acgaatgatggcgtcacgaccatcgctaataaccttaccagcacggttcaagtcttctcggactcggagt |
| | | accagttgccgtacgtcctcggctctgcgcaccagggctgcctccctccgttcccggcggacgtgttcat |
| | | ga |
| Chimera | 15 | aaaagactccaaatcggccgaccaacccggactctgggaaggccccggccaagaaaaagcaaaaag |
| 6.6 | | acggcgaaccagccgactctgctagaaggacactcgactttgaagactctggagcaggagacggacc |
| AAV12VP1u- | | ccctgagggatcatcttccggagaaactgctcatgatgctgagatggcttcaggcggtggcgcacc |
| AAV6VP2/3 | | aatggcagacaataacgaaggcgccgacggagtgggtaatgcctcaggaaattggcattgcgattcca |
| | | catggctgggcgacagagtcatcaccaccagcacccgaacatgggccttgcccacctataacaaccac |
| | | ctctacaagcaaatctccagtgcttcaacgggggccagcaacgacaaccactacttcggctacagcacc |
| | | ccctgggggtattttgatttcaacagattccactgccatttctcaccacgtgactggcagcgactcatcaac |
| | | aacaattggggattccggcccaagagactcaacttcaagctcttcaacatccaagtcaaggaggtcacg |
| | | acgaatgatggcgtcacgaccatcgctaataaccttaccagcacggttcaagtcttctcggactcggagt |
| | | accagttgccgtacgtcctcggctctgcgcaccagggctgcctccctccgttcccggcggacgtgttcat |
| | | ga |

TABLE 5
AAP Amino Acid Sequences of Chimeras (amino acids 13-27 of
AAV6 AAP or corresponding amino acids in AAP of Chimera 6,
6.1, 6.2, 6.3, 6.4, 6.5 and 6.6 are underlined; SEQ ID NO: 9
is the same for WT AAV6 and Chimeras 2, 7, and 8)

| | SEQ ID | |
|---|---|---|
| Chimera | NO: | AAP amino acid sequence |
| Chimera 2 | 2 | LATQSQSPTHNLSENLQQPPLLWDLLQWLQAVAHQWQTITKAP |
| AAV5VP1u- | | TEWVMPQEIGIAIPHGWATESSPPAPEHGPCPPITTTSTSKSPVLQ |
| AAV6VP2/3 | | RGPATTTTTSATAPPGGILISTDSTAISHHVTGSDSSTTIGDSGPR |
| | | DSTSSSSTSKSRRSRRMMASRPSLITLPARFKSSRTRSTSCRTSSA |
| | | LRTRAASLRSRRTCS |
| Chimera 7 | 2 | LATQSQSPTHNLSENLQQPPLLWDLLQWLQAVAHQWQTITKAP |
| AAV4VP1u- | | TEWVMPQEIGIAIPHGWATESSPPAPEHGPCPPITTTSTSKSPVLQ |
| AAV6VP2/3 | | RGPATTTTTSATAPPGGILISTDSTAISHHVTGSDSSTTIGDSGPR |
| | | DSTSSSSTSKSRRSRRMMASRPSLITLPARFKSSRTRSTSCRTSSA |
| | | LRTRAASLRSRRTCS |
| Chimera 8 | 2 | LATQSQSPTHNLSENLQQPPLLWDLLQWLQAVAHQWQTITKAP |
| AAV12VP1u- | | TEWVMPQEIGIAIPHGWATESSPPAPEHGPCPPITTTSTSKSPVLQ |
| AAV6VP2/3 | | RGPATTTTTSATAPPGGILISTDSTAISHHVTGSDSSTTIGDSGPR |
| | | DSTSSSSTSKSRRSRRMMASRPSLITLPARFKSSRTRSTSCRTSSA |
| | | LRTRAASLRSRRTCS |
| Chimera 3 | 16 | LNPPSSPTPPRVSAKKASSRLKRSSFSKTKLEQATDPLRDQLPEP |
| rAAV4P1/2- | | CLMTVRWLQAVAHQWQTITKAPTEWVMPQEIGIAIPHGWATE |
| AAV6VP3 | | SSPPAPEHGPCPPITTTSTSKSPVLQRGPATTTTTSATAPPGGILIS |
| | | TDSTAISHHVTGSDSSTTIGDSGPRDSTSSSSTSKSRRSRRMMAS |
| | | RPSLITLPARFKSSRTRSTSCRTSSALRTRAASLRSRRTCS |
| Chimera 4 | 17 | TTTFQKERRLGPKRTPSLPPRQTPKLDPADPSSCKSQPNQPQVW |
| rAAV5VP1/2- | | ELIQWLQAVAHQWQTITKAPTEWVMPQEIGIAIPHGWATESSPP |
| AAV6VP3 | | APEHGPCPPITTTSTSKSPVLQRGPATTTTTSATAPPGGILISTDST |
| | | AISHHVTGSDSSTTIGDSGPRDSTSSSSTSKSRRSRRMMASRPSLI |
| | | TLPARFKSSRTRSTSCRTSSALRTRAASLRSRRTCS |
| Chimera 5 | 18 | SHHKSPTPPRASAKKANNQPERGSTLKRTLEPETDPLKDQIPAP |
| rAAV11VP1/2- | | CLQTLKWLQAVAHQWQTITKAPTEWVMPQEIGIAIPHGWATES |
| AAV6VP3 | | SPPAPEHGPCPPITTTSTSKSPVLQRGPATTTTTSATAPPGGILIST |
| | | DSTAISHHVTGSDSSTTIGDSGPRDSTSSSSTSKSRRSRRMMASR |
| | | PSLITLPARFKSSRTRSTSCRTSSALRTRAASLRSRRTCS |
| Chimera 6 | 19 | KRLQIGRPTRTLGRPRPRKSKKTANQPTLLEGHSTLKTLEQETD |
| AAV12VP1/2- | | PLRDHLPEKCLMMLRWLQAVAHQWQTITKAPTEWVMPQEIGI |
| AAV6VP3 | | AIPHGWATESSPPAPEHGPCPPITTTSTSKSPVLQRGPATTTTTSA |
| | | TAPPGGILISTDSTAISHHVTGSDSSTTIGDSGPRDSTSSSSTSKSR |
| | | RSRRMMASRPSLITLPARFKSSRTRSTSCRTSSALRTRAASLRSR |
| | | RTCS |
| Chimera | 20 | KRLQIGRPTRTLGRPRPRKSKKTANQPTLLEGHSTLKTLEQETD |
| 6.1 | | SENLQQPPLLWDLLQWLQAVAHQWQTITKAPTEWVMPQEIGI |
| AAV12VP1/2- | | AIPHGWATESSPPAPEHGPCPPITTTSTSKSPVLQRGPATTTTTSA |
| AAV6VP3 | | TAPPGGILISTDSTAISHHVTGSDSSTTIGDSGPRDSTSSSSTSKSR |
| | | RSRRMMASRPSLITLPARFKSSRTRSTSCRTSSALRTRAASLRSR |
| | | RTCS |
| Chimera | 21 | KRLQIGRPTRTLGRPRPRKSKKTANQPTLLEGHSTLKTLEQETD |
| 6.2 | | SENLQQPPKCLMMLRWLQAVAHQWQTITKAPTEWVMPQEIGI |
| AAV12VP1/2- | | AIPHGWATESSPPAPEHGPCPPITTTSTSKSPVLQRGPATTTTTSA |
| AAV6VP3 | | TAPPGGILISTDSTAISHHVTGSDSSTTIGDSGPRDSTSSSSTSKSR |
| | | RSRRMMASRPSLITLPARFKSSRTRSTSCRTSSALRTRAASLRSR |
| | | RTCS |
| Chimera | 22 | KRLQIGRPTRTLGRPRPRKSKKTANQPTLLEGHSTLKTLEQETD |
| 6.3 | | PLRDHLPELLWDLLQWLQAVAHQWQTITKAPTEWVMPQEIGI |
| AAV12VP1/2- | | AIPHGWATESSPPAPEHGPCPPITTTSTSKSPVLQRGPATTTTTSA |
| AAV6VP3 | | TAPPGGILISTDSTAISHHVTGSDSSTTIGDSGPRDSTSSSSTSKSR |
| | | RSRRMMASRPSLITLPARFKSSRTRSTSCRTSSALRTRAASLRSR |
| | | RTCS |
| Chimera | 23 | KRLQIGRPTRTLGRPRPRKSKKTANQPTLLEGHSTLKTLEQETD |
| 6.4 | | PLRDHLPEKCLMMLQWLQAVAHQWQTITKAPTEWVMPQEIGI |
| AAV12VP1/2- | | AIPHGWATESSPPAPEHGPCPPITTTSTSKSPVLQRGPATTTTTSA |
| AAV6VP3 | | TAPPGGILISTDSTAISHHVTGSDSSTTIGDSGPRDSTSSSSTSKSR |
| | | RSRRMMASRPSLITLPARFKSSRTRSTSCRTSSALRTRAASLRSR |
| | | RTCS |
| Chimera | 24 | KRLQIGRPTRTLGRPRPRKSKKTANQPTLLEGHSTLKTLEQETD |
| 6.5 | | PLRDHLPEKCLDMLRWLQAVAHQWQTITKAPTEWVMPQEIGI |
| AAV12VP1/2- | | AIPHGWATESSPPAPEHGPCPPITTTSTSKSPVLQRGPATTTTTSA |
| AAV6VP3 | | TAPPGGILISTDSTAISHHVTGSDSSTTIGDSGPRDSTSSSSTSKSR |
| | | RSRRMMASRPSLITLPARFKSSRTRSTSCRTSSALRTRAASLRSR |
| | | RTCS |
| Chimera | 25 | KRLQIGRPTRTLGRPRPRKSKKTANQPTLLEGHSTLKTLEQETD |
| 6.6 | | PLRDHLPEKLLMMLRWLQAVAHQWQTITKAPTEWVMPQEIGI |
| AAV12VP1/2- | | AIPHGWATESSPPAPEHGPCPPITTTSTSKSPVLQRGPATTTTTSA |
| AAV6VP3 | | TAPPGGILISTDSTAISHHVTGSDSSTTIGDSGPRDSTSSSSTSKSR |
| | | RSRRMMASRPSLITLPARFKSSRTRSTSCRTSSALRTRAASLRSR |
| | | RTCS |

TABLE 6
WT AAV alternative reading frame (AAP) amino acid and nucleic
acid sequences

| | SEQ ID | | SEQ ID | |
|---|---|---|---|---|
| Construct | NO | Amino acid sequence | NO | Nucleic acid sequence |
| AAV6 | 1 | LATQSQSPTHNLSENLQQ | 2 | ctggcgactcagagtcagtccccga |
| | | PPLLWDLLQWLQAVAHQ | | cccacaacctctcggagaacctcca |
| | | WQTITKAPTEWVMPQEI | | gcaacccccgctgctgtgggaccta |
| | | GIAIPHGWATESSPPAPEH | | ctacaatggcttcaggcggtggcgc |
| | | GPCPPITTTSTSKSPVLQR | | accaatggcagacaataacgaagg |
| | | GPATTTTTSATAPPGGILI | | cgccgacggagtgggtaatgcctca |
| | | STDSTAISHHVTGSDSSTT | | ggaaattggcattgcgattccacatg |
| | | IGDSGPRDSTSSSSTSKSR | | gctgggcgacagagtcatcaccacc |
| | | RSRRMMASRPSLITLPAR | | agcacccgaacatgggccttgccca |
| | | FKSSRTRSTSCRTSSALRT | | cctataacaaccacctctacaagcaa |
| | | RAASLRSRRTCS | | atctccagtgcttcaacgggggcca |
| | | | | gcaacgacaaccactacttcggcta |
| | | | | cagcaccccctgggggtattttgattt |
| | | | | caacagattccactgccatttctcacc |
| | | | | acgtgactggcagcgactcatcaac |
| | | | | aacaattggggattccggcccaaga |
| | | | | gactcaacttcaagctcttcaacatcc |
| | | | | aagtcaaggaggtcacgacgaatga |
| | | | | tggcgtcacgaccatcgctaataacc |
| | | | | ttaccagcacggttcaagtcttctcgg |
| | | | | actcggagtaccagttgccgtacgtc |
| | | | | ctcggctctgcgcaccagggctgcc |
| | | | | tccctccgttcccggcggacgtgttc |
| | | | | atga |
| AAV4 | 36 | LNPPSSPTPPRVSAKKASS | 40 | ttgaatccccccagcagcccgactc |
| | | RLKRSSFSKTKLEQATDP | | ctccacgggtatcggcaaaaaaggc |
| | | LRDQLPEPCLMTVRCVQ | | aagcagccggctaaaaagaagctc |
| | | QLAELQSRADKVPMEWV | | gattcgaagacgaaactggagcag |
| | | MPRVIGIAIPPGLRATSRP | | gcgacggaccccctgagggatcaa |
| | | PAPEPGSCPPTTTTSTSDS | | cttccggagccatgtctgatgacagt |
| | | ERACSPTPTTDSPPPGDTL | | gagatgcgtgcagcagctggcgga |
| | | TSTASTATSHHVTGSDSS | | gctgcagtcgagggcggacaaggt |
| | | TTTGACDPKPCGSKS STS | | gccgatggagtgggtaatgcctcgg |
| | | RSRRSRRRTARQRWLITL | | gtgattggcattgcgattccacctggt |
| | | PARFRSLRTRRTNCRT | | ctgagggccacgtcacgaccacca |
| | | | | gcaccagaacctgggtcttgcccac |
| | | | | ctacaacaaccacctctacaagcga |
| | | | | ctcggagagagcctgcagtccaaca |
| | | | | cctacaacggattctccaccccctgg |
| | | | | ggatactttgacttcaaccgcttccac |
| | | | | tgccacttctcaccacgtgactggca |
| | | | | gcgactcatcaacaacaactggggc |
| | | | | atgcgacccaaagccatgcgggtca |
| | | | | aaatcttcaacatccaggtcaaggag |
| | | | | gtcacgacgtcgaacggcgagaca |
| | | | | acggtggctaataaccttaccagcac |
| | | | | ggttcagatctttgcggactcgtcgta |
| | | | | cgaactgccgtacgtga |
| AAV5 | 37 | TTTFQKERRLGPKRTPSL | 41 | acgaccactttccaaaaagaaagaa |
| | | PPRQTPKLDPADPSSCKS | | ggctcggaccgaagaggactccaa |
| | | QPNQPQVWELIQCLREV | | gccttccacctcgtcagacgccgaa |
| | | AAHWATITKVPMEWAM | | gctggacccagcggatcccagcag |
| | | PREIGIAIPRGWGTESSPS | | ctgcaaatcccagcccaaccagcct |
| | | PPEPGCCPATTTTSTERSK | | caagtttgggagctgatacaatgtct |
| | | AAPSTEATPTPTLDTAPP | | gcgggaggtggcggcccattgggc |
| | | GGTLTLTASTATGAPETG | | gacaataaccaaggtgccgatggag |
| | | KDSSTTTGASDPGPSESK | | tgggcaatgcctcgggagattggca |
| | | SSTFKSKRSRCRTPPPPSP | | ttgcgattccacgtggatgggggac |
| | | TTSPPPSKCLRTTTTSCPT | | agagtcgtcaccaagtccacccgaa |
| | | SSATGPRDACRPSLRRSL | | cctgggtgctgcccagctacaacaa |
| | | RCRSTVTRR | | ccaccagtaccgagagatcaaaagc |
| | | | | ggctccgtcgacggaagcaacgcc |
| | | | | aacgcctactttggatacagcacccc |
| | | | | ctgggggtactttgactttaaccgctt |
| | | | | ccacagccactggagcccccgaga |
| | | | | ctggcaaagactcatcaacaactact |
| | | | | ggggcttcagaccccggtccctcag |
| | | | | agtcaaaatcttcaacattcaagtcaa |
| | | | | agaggtcacggtgcaggactccacc |
| | | | | accaccatcgccaacaacctcacctc |
| | | | | caccgtccaagtgtttacggacgac |
| | | | | gactaccagctgccctacgtcgtcg |
| | | | | gcaacgggaccgagggatgcctgc |
| | | | | cggccttccctccgcaggtctttacg |
| | | | | ctgccgcagtacggttacgcgacgc |
| | | | | tga |
| AAV11 | 38 | SHHKSPTPPRASAKKANN | 42 | agtcaccacaagagcccgactcctc |
| | | QPERGSTLKRTLEPETDP | | ctcgggcatcggcaaaaaaggcaa |
| | | LKDQIPAPCLQTLKCVQH | | acaaccagccagaaagaggctcaa |
| | | RAEMLSMRDKVPMEWV | | ctttgaagaggacactggagccgga |
| | | MPRVIGIAIPPGLRARSQQ | | gacggaccccctgaaggatcagata |
| | | PRPEPGSCPPTTTTCTCVS | | ccagcgccatgtcttcagacattgaa |
| | | EQHQAATPTTDSPPPGDI | | atgcgtgcagcaccgggcggaaat |
| | | LTSTDSTVTSHHVTGKDS | | gctgtcgatgcgggacaaggttccg |
| | | STTTGDYDQKPCALKSSI | | atggagtgggtaatgcctcgggtgat |
| | | SKLRRSQRRTARLRSLITL | | tggcattgcgattccacctggtctga |
| | | PARFRYLRTRRMSSRT | | gggcaaggtcacaacaacctcgac |
| | | | | cagaacctgggtcttgcccacctaca |
| | | | | acaaccacttgtacctgcgtctcgga |
| | | | | acaacatcaagcagcaacacctaca |
| | | | | acggattctccaccccctggggatat |
| | | | | tttgacttcaacagattccactgtcact |
| | | | | tctcaccacgtgactggcaaagactc |
| | | | | atcaacaacaactggggactacgac |
| | | | | caaaagccatgcgcgttaaaatcttc |
| | | | | aatatccaagttaaggaggtcacaac |
| | | | | gtcgaacggcgagactacggtcgct |
| | | | | aataaccttaccagcacggttcagat |
| | | | | atttgcggactcgtcgtatgagctccc |
| | | | | gtacgtga |
| AAV12 | 39 | KRLQIGRPTRTLGRPRPR | 43 | aaaagactccaaatcggccgaccaa |
| | | KSKKTANQPTLLEGHSTL | | cccggactctgggaaggccccggc |
| | | KTLEQETDPLRDHLPEKC | | caagaaaaagcaaaaagacggcga |
| | | LMMLRCVRRQAEMLSRR | | accagccgactctgctagaaggaca |
| | | DKVPMEWVMPPVIGIAIP | | ctcgactttgaagactctggagcagg |
| | | PGQRAESPPPAPEPGSYP | | agacggaccccctgagggatcatct |
| | | RTTTTCTCESEQRPTATP | | tccggagaaatgtctcatgatgctga |
| | | TTDSPPPGDTLTLTASTA | | gatgcgtgcggcgccaggcggaaa |
| | | TFPHATGSDSSTTTGDSG | | tgctgtcgaggcgggacaaggtgcc |
| | | RNRCVLKSSTYRSRRSRR | | gatggagtgggtaatgcctccggtg |
| | | QTARLRSLITLPARFRSLR | | attggcattgcgattccacctggtcag |
| | | IRRMNSHT | | agggccgagtcaccaccaccagca |
| | | | | cccgaacctgggtcctacccacgta |
| | | | | caacaaccacctgtacctgcgaatc |
| | | | | ggaacaacggccaacagcaacacc |
| | | | | tacaacggattctccaccccctgggg |
| | | | | atactttgactttaaccgcttccactgc |
| | | | | cacttttccccacgcgactggcagcg |
| | | | | actcatcaacaacaactggggactc |
| | | | | aggccgaaatcgatgcgtgttaaaat |
| | | | | cttcaacatacaggtcaaggaggtca |
| | | | | cgacgtcaaacggcgagactacggt |
| | | | | cgctaataaccttaccagcacggttc |
| | | | | agatctttgcggattcgacgtatgaac |
| | | | | tcccatacgtga |

### Example 2—Viral Titer of Chimeras 6, 6.1, 6.2, 6.3, 6.4, 6.5, and 6.6

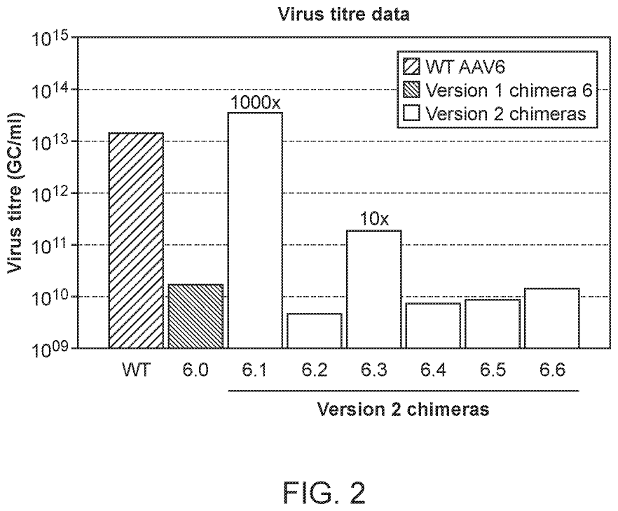

The AAV vectors containing AAV chimera 6, 6.1, 6.2, 6.3, 6.4, 6.5, or 6.6 were transformed into One Shot TOP10 Chemically Competent E. coli (Thermo Fisher). One mg of plasmid DNA for each vector was purified from the bacteria using the EndoFree Plasmid Maxi Kit (Qiagen) and sent to Vigene Biosciences, MD USA, for production of infectious AAV. The titer of the purified virus was determined (FIG. 2).

The virus titer data show that chimera 6.1 has a viral titer that is similar to WT AAV6, which is about 1000× higher than chimera 6, as shown in FIG. 2. The virus titer data also show that chimera 6.3 has a titer that is about 10× greater than chimera 6, as shown in FIG. 2.

### Example 3—T-Cells Transduced with Chimeras 6, 6.1, and 6.3

To determine how chimera 6, chimera 6.1, and chimera 6.3 each compares to WT AAV6 at a MOI of 1e6, 1e5, and 1e4 GC (genome copies)/mL in terms of infectivity, T-cells were infected with WT AAV6, chimera 6, chimera 6.1, or chimera 6.3 (CMV NanoLuc virus) at an MOI of 1e6, 1e5, or 1e4 GC/mL.

NanoLuc results in FIG. 3 show that, at a MOI of 1e4 GC/mL, chimera 6 (about 100×) and chimera 6.3 (about 10×) have increased luminescence (RLU), indicating superior infectivity in T-cells, as compared to WT AAV6. NanoLuc results in FIG. 3 also show that, at a MOI of 1e5 GC/mL, chimera 6.3 (about 100×) shows increased luminescence (RLU), indicating superior infectivity in T-cells, as compared to WT AAV6. Chimera 6.1 shows similar (at MOIs of 1e5 and 1e6 GC/mL) or slightly higher (at a MOI of 1e4 GC/mL) infectivity in T-cells, as compared to WT AAV6, as shown in NanoLuc results in FIG. 3.

### Example 4—Viral Titer of Chimera 6 Produced in the Presence of WT AAV6 AAP

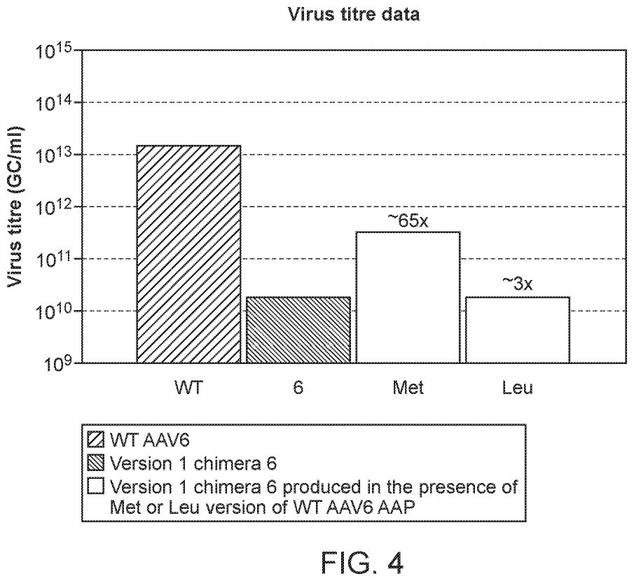

The AAV vector plasmids containing AAV chimera 6 either produced with or without the presence of Met or Leu versions of WT AAV6 AAP (Met and Leu versions only differ in their start codon) were transformed into One Shot TOP10 Chemically Competent E. coli (Thermo Fisher). One mg of plasmid DNA for each vector was purified from the bacteria using the EndoFree Plasmid Maxi Kit (Qiagen) and sent to Vigene Biosciences, MD USA, for production of infectious AAV. The titer of the purified virus was then determined (FIG. 4).

Vigene virus titer data show that chimera 6 produced in the presence of the Met version of WT AAV6 AAP has about 65× higher virus titer than chimera 6, as shown in FIG. 4. Vigene virus titer data also show that chimera 6 produced in the presence of the Leu version of WT AAV6 has about 3× higher virus titer than chimera 6, as shown in FIG. 4.

### Example 5—T-Cells Transduced with Chimera 6 in the Presence of WT AAV6 AAP

To determine how chimera 6 (alone) or chimera 6 plus a WT AAV6 AAP sequence in trans (either Met or Leu version; Met and Leu versions only differ in their start codon) compares to WT AAV6 at a MOI of 1e4 GC/mL in terms of infectivity, T-cells were infected with WT AAV6, chimera 6, or chimera 6 with a trans WT AAV6 AAP (CMV NanoLuc virus) at a MOI of 1e4 GC/mL.

NanoLuc results show that, as compared to WT, both chimera 6 (about 100×) and chimera 6 produced in the presence of WT AAV6 AAP (about 100× for the Met version and about 10× for the Leu version) show increased luminescence (RLU), or superior infectivity in T-cells, as shown in FIG. 5.

1. A polynucleic acid sequence that encodes:
a. in a first reading frame, an adeno-associated virus (AAV) VP1 polypeptide, an AAV VP2 polypeptide, and an AAV VP3 polypeptide, and
b. in a second reading frame, a modified AAV assembly-activating protein (AAP) polypeptide that is at least partially in a region of said first reading frame that encodes at least a portion of said VP2 polypeptide and at least a portion of said VP3 polypeptide, and
wherein said AAP polypeptide comprises i) at least one amino acid substitution in said region of said first reading frame that encodes at least a portion of said VP2 polypeptide as compared to a wild-type AAV AAP polypeptide of the same AAV serotype of said VP2 polypeptide; or ii) at least one amino acid substitution in said region of said first reading frame that encodes at least a portion of said VP3 polypeptide as compared to a wild-type AAV AAP polypeptide of the same AAV serotype of said VP3 polypeptide,
and wherein one of said VP1, VP2, and VP3 polypeptides is a first AAV serotype, and one of said VP1, VP2, and VP3 polypeptides is a second AAV serotype, wherein said first and second AAV serotypes are different.

2. (canceled)

3. The polynucleic acid sequence of claim 1, wherein introduction of a said polynucleic acid into a population of cells under conditions suitable for AAV particle production from said cells, results in a higher titer of AAV particles produced by said population of cells compared to introduction of a comparable polynucleic acid lacking said modified AAP polypeptide.

4.-6. (canceled)

7. The polynucleic acid sequence of claim 1, wherein said VP2 polypeptide is an AAV6 serotype, and said at least one amino acid substitution in said region of said first reading frame that encodes at least a portion of said VP2 polypeptide is in a helical region of said modified AAP polypeptide is within amino acids 13 to 27 of said AAP polypeptide.

8. The polynucleic acid sequence of claim 7, wherein said at least one amino acid substitution in said region of said first reading frame that encodes at least a portion of said VP2 polypeptide is in a helical region of said modified AAP polypeptide is within amino acids 21 to 27 of said AAP polypeptide.

9. The polynucleic acid of claim 1, wherein said at least one amino acid substitution comprises a substitution at amino acid K53, C54, L55, M56, M57, or R59 of SEQ ID NO: 39, or any combination thereof, in said AAP polypeptide.

10.-16. (canceled)

17. The polynucleic acid sequence of claim 1, wherein said first AAV serotype and said second AAV serotype are selected from AAV1, AAV2, AAV3, AAV4, AAV5, AAV6, AAV7, AAV8, AAV9, AAV10, AAV11, and AAV12.

18. The polynucleic acid sequence of claim 1, wherein said first AAV serotype is AAV12 and said second AAV serotype is AAV6.

19. The polynucleic acid sequence of claim 1, wherein said VP1 and VP2 polypeptides are AAV12 serotype and said VP3 polypeptide is an AAV6 serotype.

20. A polynucleic acid sequence that encodes i) in a first reading frame, a VP2 polypeptide of an AAV serotype, and ii) in a second reading frame, a modified assembly-activating protein (AAP) polypeptide comprising at least one amino acid substitution within amino acids 5-40 in said modified AAP polypeptide with respect to a wild type AAP polypeptide of the AAV serotype.

21. The polynucleic acid sequence of claim 20, wherein said polynucleic acid sequence comprises a nucleic acid sequence encoding an AAV12 VP1 polypeptide, a nucleic acid sequence encoding an AAV12 VP2 polypeptide, and a nucleic acid sequence encoding an AAV6 VP3 polypeptide, in a single reading frame.

22. The polynucleic acid sequence of claim 20, wherein said at least one amino acid substitution comprises a substitution at amino acid K53, C54, L55, M56, M57, and R59 of SEQ ID NO: 39, or any combination thereof, in said AAP polypeptide.

23.-29. (canceled)

30. The polynucleic acid sequence of claim 20, wherein said AAV serotype is AAV6.

31.-67. (canceled)

68. A system comprising a first polynucleic acid sequence that encodes at least three adeno-associated virus (AAV) polypeptides, wherein said first polynucleic acid sequence encodes a VP1 polypeptide, a VP2 polypeptide, and a VP3 polypeptide, wherein two of said VP1, VP2, and VP3 polypeptides are from a first AAV serotype, and one of said VP1, VP2, and VP3 polypeptides is from a second AAV serotype, wherein said first AAV serotype and said second AAV serotype are not the same; and a second polynucleic acid sequence heterologous to said first polynucleic acid sequence that encodes an AAV assembly-activating protein (AAP) polypeptide, wherein said first polynucleic acid sequence and second polynucleic acid sequence are not covalently linked.

69.-70. (canceled)

71. The system of claim 68, wherein said AAV AAP polypeptide is an AAV6 AAP polypeptide.

72. The system of claim 68, wherein said first AAV serotype and said second AAV serotype are selected from AAV1, AAV2, AAV3, AAV4, AAV5, AAV6, AAV7, AAV8, AAV9, AAV10, AAV11, AAV12, or any combination thereof.

73. The system of claim 68, wherein said first AAV serotype is AAV12.

74. The system of claim 68, wherein said first AAV serotype is AAV12 and said second AAV serotype is AAV6.

75. The system of claim 74, wherein said first polynucleic acid sequence encodes an AAV12 VP1, an AAV12, VP2, and an AAV6 VP3.

76.-111. (canceled)

112. The system of claim 68, further comprising a third polynucleic acid sequence that encodes a Rep polypeptide.

113. The system of claim 112, wherein the Rep polypeptide comprises a modified Rep polypeptide, and wherein the modified Rep polypeptide provides at least one of improved packaging efficiency, yield, infectivity, transduction efficiency, and transfection efficiency as compared to a system lacking said modified Rep polypeptide.


SEQUENCE LISTING (72 sequences)

SEQ ID NO: 1 - DNA, 591 bp, Adeno-associated virus
ctggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 2 - protein, 196 aa, Unknown
  Description of Unknown Adeno-associated virus or chimeric sequence
LATQSQSPTHNLSENLQQPPLLWDLLQWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 3 - DNA, 591 bp, Artificial Sequence; identical to SEQ ID NO: 1
  Description of Artificial Sequence Synthetic polynucleotide

SEQ ID NO: 4 - DNA, 663 bp, Artificial Sequence
  Description of Artificial Sequence Synthetic polynucleotide
ttgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 5 - DNA, 654 bp, Artificial Sequence
  Description of Artificial Sequence Synthetic polynucleotide
acgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 6 - DNA, 660 bp, Artificial Sequence
  Description of Artificial Sequence Synthetic polynucleotide
agtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 7 - DNA, 687 bp, Artificial Sequence
  Description of Artificial Sequence Synthetic polynucleotide
aaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 8 - DNA, 591 bp, Artificial Sequence; identical to SEQ ID NO: 1
  Description of Artificial Sequence Synthetic polynucleotide

SEQ ID NO: 9 - DNA, 591 bp, Artificial Sequence; identical to SEQ ID NO: 1
  Description of Artificial Sequence Synthetic polynucleotide

SEQ ID NO: 10 - DNA, 687 bp, Artificial Sequence
  Description of Artificial Sequence Synthetic polynucleotide
aaaagactccaaatcggccgaccaacccggactctgggaaggccccggccaagaaaaagcaaaaagacggcgaaccagccgactctgctagaaggacactcgactttgaagactctggagcaggagacggactcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 11 - DNA, 687 bp, Artificial Sequence
  Description of Artificial Sequence Synthetic polynucleotide
aaaagactccaaatcggccgaccaacccggactctgggaaggccccggccaagaaaaagcaaaaagacggcgaaccagccgactctgctagaaggacactcgactttgaagactctggagcaggagacggactcggagaacctccagcaacccccgaaatgtctcatgatgctgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 12 - DNA, 687 bp, Artificial Sequence
  Description of Artificial Sequence Synthetic polynucleotide
aaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 13 - DNA, 687 bp, Artificial Sequence
  Description of Artificial Sequence Synthetic polynucleotide
aaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgcaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 14 - DNA, 687 bp, Artificial Sequence
  Description of Artificial Sequence Synthetic polynucleotide
aaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcgacatgctgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 15 - DNA, 687 bp, Artificial Sequence
  Description of Artificial Sequence Synthetic polynucleotide
aaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaactgctcatgatgctgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatga

SEQ ID NO: 16 - protein, 220 aa, Artificial Sequence
  Description of Artificial Sequence Synthetic polypeptide
LNPPSSPTPPRVSAKKASSRLKRSSFSKTKLEQATDPLRDQLPEPCLMTVRWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 17 - protein, 217 aa, Artificial Sequence
  Description of Artificial Sequence Synthetic polypeptide
TTTFQKERRLGPKRTPSLPPRQTPKLDPADPSSCKSQPNQPQVWELIQWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 18 - protein, 219 aa, Artificial Sequence
  Description of Artificial Sequence Synthetic polypeptide
SHHKSPTPPRASAKKANNQPERGSTLKRTLEPETDPLKDQIPAPCLQTLKWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 19 - protein, 228 aa, Artificial Sequence
  Description of Artificial Sequence Synthetic polypeptide
KRLQIGRPTRTLGRPRPRKSKKTANQPTLLEGHSTLKTLEQETDPLRDHLPEKCLMMLRWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 20 - protein, 228 aa, Artificial Sequence
  Description of Artificial Sequence Synthetic polypeptide
KRLQIGRPTRTLGRPRPRKSKKTANQPTLLEGHSTLKTLEQETDSENLQQPPLLWDLLQWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 21 - protein, 228 aa, Artificial Sequence
  Description of Artificial Sequence Synthetic polypeptide
KRLQIGRPTRTLGRPRPRKSKKTANQPTLLEGHSTLKTLEQETDSENLQQPPKCLMMLRWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 22 - protein, 228 aa, Artificial Sequence
  Description of Artificial Sequence Synthetic polypeptide
KRLQIGRPTRTLGRPRPRKSKKTANQPTLLEGHSTLKTLEQETDPLRDHLPELLWDLLQWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 23 - protein, 228 aa, Artificial Sequence
  Description of Artificial Sequence Synthetic polypeptide
KRLQIGRPTRTLGRPRPRKSKKTANQPTLLEGHSTLKTLEQETDPLRDHLPEKCLMMLQWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 24 - protein, 228 aa, Artificial Sequence
  Description of Artificial Sequence Synthetic polypeptide
KRLQIGRPTRTLGRPRPRKSKKTANQPTLLEGHSTLKTLEQETDPLRDHLPEKCLDMLRWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 25 - protein, 228 aa, Artificial Sequence
  Description of Artificial Sequence Synthetic polypeptide
KRLQIGRPTRTLGRPRPRKSKKTANQPTLLEGHSTLKTLEQETDPLRDHLPEKLLMMLRWLQAVAHQWQTITKAPTEWVMPQEIGIAIPHGWATESSPPAPEHGPCPPITTTSTSKSPVLQRGPATTTTTSATAPPGGILISTDSTAISHHVTGSDSSTTIGDSGPRDSTSSSSTSKSRRSRRMMASRPSLITLPARFKSSRTRSTSCRTSSALRTRAASLRSRRTCS

SEQ ID NO: 26 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 27 - protein, 734 aa, Adeno-associated virus
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 28 - protein, 724 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 29 - protein, 733 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFECANNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 30 - protein, 742 aa, Adeno-associated virus
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 31 - DNA, 2211 bp, Adeno-associated virus
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 32 - DNA, 2205 bp, Adeno-associated virus
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 33 - DNA, 2175 bp, Adeno-associated virus
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 34 - DNA, 2202 bp, Adeno-associated virus
atggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtgttcatggtgcctcaatatggctactgtggcatcgtgactggcgagaatcagaaccaaacggacagaaacgctttctactgcctggagtattttccttcgcaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcatcacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaa

SEQ ID NO: 35 - DNA, 2229 bp, Adeno-associated virus
atggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatgcgtgcggcgccaggcggaaatgctgtcgaggcgggacaaggtgccgatggagtgggtaatgcctccggtgattggcattgcgattccacctggtcagagggccgagtcaccaccaccagcacccgaacctgggtcctacccacgtacaacaaccacctgtacctgcgaatcggaacaacggccaacagcaacacctacaacggattctccaccccctggggatactttgactttaaccgcttccactgccacttttccccacgcgactggcagcgactcatcaacaacaactggggactcaggccgaaatcgatgcgtgttaaaatcttcaacatacaggtcaaggaggtcacgacgtcaaacggcgagactacggtcgctaataaccttaccagcacggttcagatctttgcggattcgacgtatgaactcccatacgtgatggacgccggtcaggaggggagctttcctccgtttcccaacgacgtctttatggttccccaatacggatactgcggagttgtcactggaaaaaaccagaaccagacagacagaaatgccttttactgcctggaatactttccatcccaaatgctaagaactggcaacaattttgaagtcagttaccaatttgaaaaagttcctttccattcaatgtacgcgcacagccagagcctggacagaatgatgaatcctttactggatcagtacctgtggcatctgcaatcgaccactaccggaaattcccttaatcaaggaacagctaccaccacgtacgggaaaattaccactggagactttgcctactacaggaaaaactggttgcctggagcctgcattaaacaacaaaaattttcaaagaatgccaatcaaaactacaagattcccgccagcgggggagacgcccttttaaagtatgacacgcataccactctaaatgggcgatggagtaacatggctcctggacctccaatggcaaccgcaggtgccggggactcggattttagcaacagccagctgatctttgccggacccaatccgagcggtaacacgaccacatcttcaaacaatttgttgtttacctcagaagaggagattgccacaacaaacccacgagacacggacatgtttggacagattgcagataataatcaaaatgccaccaccgcccctcacatcgctaacctggacgctatgggaattgttcccggaatggtctggcaaaacagagacatctactaccagggccctatttgggccaaggtccctcacacggacggacactttcacccttcgccgctgatgggaggatttggactgaaacacccgcctccacagattttcatcaaaaacacccccgtacccgccaatcccaatactacctttagcgctgcaaggattaattcttttctgacgcagtacagcaccggacaagttgccgttcagatcgactgggaaattcagaaggagcattccaaacgctggaatcccgaagttcaatttacttcaaactacggcactcaaaattctatgctgtgggctcccgacaatgctggcaactaccacgaactccgggctattgggtcccgtttcctcacccaccacttgtaa

SEQ ID NO: 36 - protein, 198 aa, Adeno-associated virus
LNPPSSPTPPRVSAKKASSRLKRSSFSKTKLEQATDPLRDQLPEPCLMTVRCVQQLAELQSRADKVPMEWVMPRVIGIAIPPGLRATSRPPAPEPGSCPPTTTTSTSDSERACSPTPTTDSPPPGDTLTSTASTATSHHVTGSDSSTTTGACDPKPCGSKSSTSRSRRSRRRTARQRWLITLPARFRSLRTRRTNCRT

SEQ ID NO: 37 - protein, 226 aa, Adeno-associated virus
TTTFQKERRLGPKRTPSLPPRQTPKLDPADPSSCKSQPNQPQVWELIQCLREVAAHWATITKVPMEWAMPREIGIAIPRGWGTESSPSPPEPGCCPATTTTSTERSKAAPSTEATPTPTLDTAPPGGTLTLTASTATGAPETGKDSSTTTGASDPGPSESKSSTFKSKRSRCRTPPPPSPTTSPPPSKCLRTTTTSCPTSSATGPRDACRPSLRRSLRCRSTVTRR

SEQ ID NO: 38 - protein, 197 aa, Adeno-associated virus
SHHKSPTPPRASAKKANNQPERGSTLKRTLEPETDPLKDQIPAPCLQTLKCVQHRAEMLSMRDKVPMEWVMPRVIGIAIPPGLRARSQQPRPEPGSCPPTTTTCTCVSEQHQAATPTTDSPPPGDILTSTDSTVTSHHVTGKDSSTTTGDYDQKPCALKSSISKLRRSQRRTARLRSLITLPARFRYLRTRRMSSRT

SEQ ID NO: 39 - protein, 206 aa, Adeno-associated virus
KRLQIGRPTRTLGRPRPRKSKKTANQPTLLEGHSTLKTLEQETDPLRDHLPEKCLMMLRCVRRQAEMLSRRDKVPMEWVMPPVIGIAIPPGQRAESPPPAPEPGSYPRTTTTCTCESEQRPTATPTTDSPPPGDTLTLTASTATFPHATGSDSSTTTGDSGRNRCVLKSSTYRSRRSRRQTARLRSLITLPARFRSLRIRRMNSHT

SEQ ID NO: 40 - DNA, 597 bp, Adeno-associated virus
ttgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtga

SEQ ID NO: 41 - DNA, 681 bp, Adeno-associated virus
acgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctga

SEQ ID NO: 42 - DNA, 594 bp, Adeno-associated virus
agtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtga

SEQ ID NO: 43 - DNA, 621 bp, Adeno-associated virus
aaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatgcgtgcggcgccaggcggaaatgctgtcgaggcgggacaaggtgccgatggagtgggtaatgcctccggtgattggcattgcgattccacctggtcagagggccgagtcaccaccaccagcacccgaacctgggtcctacccacgtacaacaaccacctgtacctgcgaatcggaacaacggccaacagcaacacctacaacggattctccaccccctggggatactttgactttaaccgcttccactgccacttttccccacgcgactggcagcgactcatcaacaacaactggggactcaggccgaaatcgatgcgtgttaaaatcttcaacatacaggtcaaggaggtcacgacgtcaaacggcgagactacggtcgctaataaccttaccagcacggttcagatctttgcggattcgacgtatgaactcccatacgtga

SEQ ID NO: 44 - protein, 735 aa, Artificial Sequence
  Description of Artificial Sequence Synthetic polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 45 - protein, 730 aa, Artificial Sequence
  Description of Artificial Sequence Synthetic polypeptide
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 46 - protein, 726 aa, Artificial Sequence
  Description of Artificial Sequence Synthetic polypeptide
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 730 aa, Artificial Sequence
  Description of Artificial Sequence Synthetic polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 48 - protein, 739 aa, Artificial Sequence
  Description of Artificial Sequence Synthetic polypeptide
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 49 - protein, 735 aa, Artificial Sequence
  Description of Artificial Sequence Synthetic polypeptide
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 50 - protein, 736 aa, Artificial Sequence
  Description of Artificial Sequence Synthetic polypeptide
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 51 - DNA, 2208 bp, Artificial Sequence
  Description of Artificial Sequence Synthetic polynucleotide
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 52 - DNA, 2192 bp, Artificial Sequence
  Description of Artificial Sequence Synthetic polynucleotide
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 53 - DNA, 2181 bp, Artificial Sequence
  Description of Artificial Sequence Synthetic polynucleotide
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 54 - DNA, 2193 bp, Artificial Sequence
  Description of Artificial Sequence Synthetic polynucleotide
atggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 55 - DNA, 2219 bp, Artificial Sequence
  Description of Artificial Sequence Synthetic polynucleotide
atggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaagaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 56 - DNA, 2200 bp, Artificial Sequence
  Description of Artificial Sequence Synthetic polynucleotide
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtc

SEQ ID NO: 57 - DNA, 2211 bp, Artificial Sequence
  Description of Artificial Sequence Synthetic polynucleotide
atggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 58 - DNA, 2529 bp, Artificial Sequence
  Description of Artificial Sequence Synthetic polynucleotide
ggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggt

SEQ ID NO: 59 - DNA, 2529 bp, Artificial Sequence
  Description of Artificial Sequence Synthetic polynucleotide
ggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggt

SEQ ID NO: 60 - DNA, 2532 bp, Artificial Sequence
  Description of Artificial Sequence Synthetic polynucleotide
ggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggt

SEQ ID NO: 61 - DNA, 2532 bp, Artificial Sequence
  Description of Artificial Sequence Synthetic polynucleotide
ggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggt

SEQ ID NO: 62 - DNA, 2514 bp, Artificial Sequence
  Description of Artificial Sequence Synthetic polynucleotide
ggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggt

SEQ ID NO: 63 - DNA, 2502 bp, Artificial Sequence
  Description of Artificial Sequence Synthetic polynucleotide
ggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggt

SEQ ID NO: 64 - DNA, 2514 bp, Artificial Sequence
  Description of Artificial Sequence Synthetic polynucleotide
ggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggt

SEQ ID NO: 65 - DNA, 2541 bp, Artificial Sequence
  Description of Artificial Sequence Synthetic polynucleotide
ggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggt

SEQ ID NO: 66 - protein, 88 aa, Adeno-associated virus
LATQSQSPTHNLSENLQQPPLLWDLLQWLQAVAHQWQTITKAPTEWVMPQEIGIAIPHGWATESSPPAPEHGPCPPITTTSTSKSPVL

SEQ ID NO: 67 - protein, 120 aa, Artificial Sequence
  Description of Artificial Sequence Synthetic polypeptide
KRLQIGRPTRTLGRPRPRKSKKTANQPTLLEGHSTLKTLEQETDSENLQQPPLLWDLLQWLQAVAHQWQTITKAPTEWVMPQEIGIAIPHGWATESSPPAPEHGPCPPITTTSTSKSPVL

SEQ ID NO: 68 - protein, 120 aa, Artificial Sequence
  Description of Artificial Sequence Synthetic polypeptide
KRLQIGRPTRTLGRPRPRKSKKTANQPTLLEGHSTLKTLEQETDSENLQQPPKCLMMLRWLQAVAHQWQTITKAPTEWVMPQEIGIAIPHGWATESSPPAPEHGPCPPITTTSTSKSPVL

SEQ ID NO: 69 - protein, 120 aa, Artificial Sequence
  Description of Artificial Sequence Synthetic polypeptide
KRLQIGRPTRTLGRPRPRKSKKTANQPTLLEGHSTLKTLEQETDPLRDHLPELLWDLLQWLQAVAHQWQTITKAPTEWVMPQEIGIAIPHGWATESSPPAPEHGPCPPITTTSTSKSPVL

SEQ ID NO: 70 - protein, 120 aa, Artificial Sequence
  Description of Artificial Sequence Synthetic polypeptide
KRLQIGRPTRTLGRPRPRKSKKTANQPTLLEGHSTLKTLEQETDPLRDHLPEKCLMMLQWLQAVAHQWQTITKAPTEWVMPQEIGIAIPHGWATESSPPAPEHGPCPPITTTSTSKSPVL

SEQ ID NO: 71 - protein, 120 aa, Artificial Sequence
  Description of Artificial Sequence Synthetic polypeptide
KRLQIGRPTRTLGRPRPRKSKKTANQPTLLEGHSTLKTLEQETDPLRDHLPEKCLDMLRWLQAVAHQWQTITKAPTEWVMPQEIGIAIPHGWATESSPPAPEHGPCPPITTTSTSKSPVL

SEQ ID NO: 72 - protein, 120 aa, Artificial Sequence
  Description of Artificial Sequence Synthetic polypeptide
KRLQIGRPTRTLGRPRPRKSKKTANQPTLLEGHSTLKTLEQETDPLRDHLPEKLLMMLRWLQAVAHQWQTITKAPTEWVMPQEIGIAIPHGWATESSPPAPEHGPCPPITTTSTSKSPVL